SEQUENCE LISTING (29 sequences)

SEQ ID NO: 1 - protein, 470 aa, mouse
MAVLVLFLCLVAFPSCVLSQVQLKESGPGLVAPSQSLSITCTVSGFSLLSYGVHWVRQPPGKGLEWLGVIWTGGTTNYNSALMSRLSISKDDSKSQVFLKMNSLQTDDTAIYYCARYYYGMDYWGQGTSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 2 - protein, 234 aa, mouse
MESQIQVFVFVFLWLSGVDGDIVMTQSHKFMSTSVGDRVSITCKASQDVRNTVAWYQQKTGQSPKLLIYSSSYRNTGVPDRFTGSGSGTDFTFTISSVQAEDLAVYFCQQHYITPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 115 aa, mouse
QVQLKESGPGLVAPSQSLSITCTVSGFSLLSYGVHWVRQPPGKGLEWLGVIWTGGTTNYNSALMSRLSISKDDSKSQVFLKMNSLQTDDTAIYYCARYYYGMDYWGQGTSVTVSS

SEQ ID NO: 4 - protein, 107 aa, mouse
DIVMTQSHKFMSTSVGDRVSITCKASQDVRNTVAWYQQKTGQSPKLLIYSSSYRNTGVPDRFTGSGSGTDFTFTISSVQAEDLAVYFCQQHYITPYTFGGGTKLEIK

SEQ ID NO: 5 - protein, 115 aa, human
QVQLQESGPGLVKPSETLSLTCTVSGFSLLSYGVHWVRQPPGKGLEWLGVIWTGGTTNYNSALMSRLTISKDDSKSTVYLKMNSLKTEDTAIYYCARYYYGMDYWGQGTSVTVSS

SEQ ID NO: 6 - protein, 115 aa, human
QVQLQESGPGLVKPSETLSLTCTVSGFSLLSYGVHWVRQPPGKGLEWLGVIWTGGTTNYNSALMSRLTISKDDSKNTVYLKMNSLKTEDTAIYYCARYYYGMDYWGQGTLVTVSS

SEQ ID NO: 7 - protein, 115 aa, human
QVQLQESGPGLVKPSETLSLTCTVSGFSLLSYGVHWVRQPPGKGLEWLGVIWTGGTTNYNSALMSRFTISKDDSKNTVYLKMNSLKTEDTAIYYCARYYYGMDYWGQGTLVTVSS

SEQ ID NO: 8 - protein, 115 aa, human
QVQLQESGPGLVKPSETLSLTCTVSGFSLLSYGVHWVRQPPGKGLEWLGVIWTGGTTNYNSALMSRFTISKDDSKNTLYLKMNSLKTEDTAIYYCARYYYGMDYWGQGTLVTVSS

SEQ ID NO: 9 - protein, 107 aa, human
DIVMTQSPSFLSASVGDRVTITCKASQDVRNTVAWYQQKTGKAPKLLIYSSSYRNTGVPDRFTGSGSGTDFTLTISSLQAEDVAVYFCQQHYITPYTFGGGTKVEIK

SEQ ID NO: 10 - protein, 107 aa, human
DIVMTQSPSSLSASVGDRVTITCKASQDVRNTVAWYQQKPGKAPKLLIYSSSYRNTGVPDRFTGSGSGTDFTLTISSLQAEDVAVYFCQQHYITPYTFGGGTKVEIK

SEQ ID NO: 11 - protein, 107 aa, human
DIQMTQSPSSLSASVGDRVTITCKASQDVRNTVAWYQQKPGKAPKLLIYSSSYRNTGVPDRFSGSGSGTDFTLTISSLQAEDVAVYFCQQHYITPYTFGGGTKVEIK

SEQ ID NO: 12 - protein, 107 aa, human
DIQMTQSPSSLSASVGDRVTITCKASQDVRNTVAWYQQKPGKAPKLLIYSSSYRNTGVPDRFSGSGSGTDFTLTISSLQAEDVAVYYCQQHYITPYTFGGGTKVEIK

SEQ ID NO: 13 - protein, 10 aa, human
GFSLLSYGVH

SEQ ID NO: 14 - protein, 16 aa, human
VIWTGGTTNYNSALMS

SEQ ID NO: 15 - protein, 7 aa, human
YYYGMDY

SEQ ID NO: 16 - protein, 11 aa, human
KASQDVRNTVA

SEQ ID NO: 17 - protein, 7 aa, human
SSSYRNT

SEQ ID NO: 18 - protein, 9 aa, human
QQHYITPYT

SEQ ID NO: 19 - DNA, 345 bp, human
caggtgcagctgcaggaatccggccctggcctggtcaagccctccgagacactgtccctgacctgcaccgtgtccggcttctccctgctgtcctacggcgtgcactgggtccgacagcctccagggaagggcctggaatggctgggcgtgatctggaccggcggcaccaccaactacaactccgccctgatgtcccggctgaccatctccaaggacgactccaagtccaccgtgtacctgaagatgaactccctgaaaaccgaggacaccgccatctactactgcgcccggtactactacggcatggactactggggccagggcacctccgtgaccgtgtcctca

SEQ ID NO: 20 - DNA, 345 bp, human
caggtgcagctgcaggaatccggccctggcctggtcaagccctccgagacactgtccctgacctgcaccgtgtccggcttctccctgctgtcctacggcgtgcactgggtccgacagcctccaggcaaaggcctggaatggctgggcgtgatctggaccggcggcaccaccaactacaactccgccctgatgtcccggctgaccatctccaaggacgactccaagaacaccgtgtacctgaagatgaactccctgaaaaccgaggacaccgccatctactactgcgcccggtactactacggcatggactactggggccagggcaccctggtcaccgtgtcctca

SEQ ID NO: 21 - DNA, 345 bp, human
caggtgcagctgcaggaatccggccctggcctggtcaagccctccgagacactgtccctgacctgcaccgtgtccggcttctccctgctgtcctacggcgtgcactgggtccgacagcctccaggcaaaggcctggaatggctgggcgtgatctggaccggcggcaccaccaactacaactccgccctgatgtcccggttcaccatctccaaggacgactccaagaacaccgtgtacctgaagatgaactccctgaaaaccgaggacaccgccatctactactgcgcccggtactactacggcatggactactggggccagggcaccctggtcaccgtgtcctca

SEQ ID NO: 22 - DNA, 345 bp, human
caggtgcagctgcaggaatccggccctggcctggtcaagccctccgagacactgtccctgacctgcaccgtgtccggcttctccctgctgtcctacggcgtgcactgggtccgacagcctccaggcaaaggcctggaatggctgggcgtgatctggaccggcggcaccaccaactacaactccgccctgatgtcccggttcaccatctccaaggacgactccaagaacaccctgtacctgaagatgaactccctgaaaaccgaggacaccgccatctactactgcgcccggtactactacggcatggactactggggccagggcaccctggtcaccgtgtcctca

SEQ ID NO: 23 - DNA, 321 bp, human
gacatcgtgatgacccagtcccccagcttcctgtccgcctccgtgggcgacagagtgaccatcacatgcaaggcctctcaggacgtgcggaacaccgtggcctggtatcagcagaaaaccggcaaggcccccaagctgctgatctactcctcctcctaccggaacaccggcgtgcccgaccggtttaccggctctggctccggcaccgactttaccctgaccatcagctccctgcaggccgaggacgtggccgtgtacttctgccagcagcactacatcaccccctacaccttcggcggaggcaccaaggtggaaataaaa

SEQ ID NO: 24 - DNA, 321 bp, human
gacatcgtgatgacccagtccccctccagcctgtccgcctctgtgggcgacagagtgaccatcacatgcaaggcctctcaggacgtgcggaacaccgtggcctggtatcagcagaagcccggcaaggcccccaagctgctgatctactcctcctcctaccggaacaccggcgtgcccgaccggtttaccggctctggctccggcaccgactttaccctgaccatcagctccctgcaggccgaggacgtggccgtgtacttctgccagcagcactacatcaccccctacaccttcggcggaggcaccaaggtggaaataaaa

SEQ ID NO: 25 - DNA, 321 bp, human
gacatccagatgacccagtccccctccagcctgtccgcctctgtgggcgacagagtgaccatcacatgcaaggcctcccaggacgtgcggaacaccgtggcctggtatcagcagaagcccggcaaggcccccaagctgctgatctactcctcctcctaccggaacaccggcgtgcccgaccggttctctggctctggaagcggcaccgactttaccctgaccatcagctccctgcaggccgaggacgtggccgtgtacttctgccagcagcactacatcaccccctacaccttcggcggaggcaccaaggtggaaataaaa

SEQ ID NO: 26 - DNA, 321 bp, human
gacatccagatgacccagtccccctccagcctgtccgcctctgtgggcgacagagtgaccatcacatgcaaggcctctcaggacgtgcggaacaccgtggcctggtatcagcagaagcccggcaaggcccccaagctgctgatctactcctcctcctaccggaacaccggcgtgcccgaccggttctctggctctggaagcggcaccgactttaccctgaccatcagctccctgcaggccgaggacgtggccgtgtactactgccagcagcactacatcaccccctacaccttcggcggaggcaccaaggtggaaataaaa

SEQ ID NO: 27 - protein, 707 aa, human
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 28 - protein, 687 aa, human
PRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 29 - protein, 20 aa, human
MSLWQPLVLVLLVLGCCFAA

(19) United States
(12) Patent Application Publication
(10) Pub. No.: US 2018/0311247 A1
(43) Pub. Date: Nov. 1, 2018

(54) COMBINATION THERAPIES FOR TREATING CANCERS

(71) Applicant: GILEAD SCIENCES, INC., Foster City, CA (US)

(72) Inventors: Julie Di Paolo, Foster City, CA (US); David Breckenridge, Foster City, CA (US); Jamie Bates, Foster City, CA (US); Daniel Tumas, Foster City, CA (US)

(21) Appl. No.: 15/764,687

(22) PCT Filed: Sep. 30, 2016
(86) PCT No.: PCT/US2016/054780

§ 371 Date: Mar. 29, 2018

Related U.S. Application Data
(60) Provisional application No. 62/236,729, filed on Oct. 2, 2015.
(60) Provisional application No. 62/236,409, filed on Oct. 2, 2015.
(60) Provisional application No. 62/236,409, filed on Oct. 2, 2015.
(60) Provisional application No. 62/236,741, filed on Oct. 2, 2015.

(51) Int. Cl. A61K 31/522; A61K 31/519; C07K 16/40; A61P 19/02; A61P 35/00

(52) U.S. Cl. CPC A61K 31/522; A61K 31/519; A61P 35/00; A61P 19/02; C07K 16/40

(57) ABSTRACT

Provided herein are methods that relate to a therapeutic strategy for treatment of cancer, and allergic, autoimmune, and inflammatory disorders including hematological malignancies. In particular, the methods include administration of a BTK inhibitor and with one or more inhibitor. For example, the one or more inhibitor may be a JAK inhibitor, a ASK1 inhibitor, a BRD4 inhibitor or an MMP9 inhibitor.

### FIELD OF THE INVENTION

The present disclosure relates generally to therapeutics and compositions for treating cancers and allergic, autoimmune, and inflammatory disorders, and more specifically to the use of Bruton's Tyrosine Kinase (BTK) inhibitors (hereinafter referred to BTK or Btk inhibitors) in combination with one or more agent which modulates Janus Kinase (JAK), Apoptosis signal-regulating kinase 1 (ASK1), bromodomain-containing proteins, or matrix metallopeptidases 9 (MMP9).

### BACKGROUND

BTK inhibitors useful in treating cancers such as hematological cancers and inflammatory conditions include those taught in U.S. Pat. No. 8,940,725 (Yamamoto et al.), U.S. 2014/0330015 Yamamoto et al.) and U.S. Pat. No. 7,514,444 (Honigberg et al.).

Janus Kinase (JAK) inhibitors are known in the art, including momelotinib, peficitinib, tofacitinib, oclacitinib, ruxolitinib, baracitinib, lestaurtinib, pacritinib, filgotinib, TG101348, JS-124, and INCB39110, CHZ868, and GSK2586184. There remains a need for beneficial combination therapies.

Mitogen-activated protein kinase (MAPK) signaling cascades couple diverse extracellular and intracellular queues to appropriate cellular stress responses, including cell growth, differentiation, inflammation, and apoptosis (Kumar, S., Boehm, J., and Lee., J. C. (2003) Nat. Rev. Drug Dis. 2:717-726; Pimienta, G., and Pascual, J. (2007) Cell Cycle, 6: 2826-2632). MAPKs exist in three groups, MAP3Ks, MAP2Ks, and MAPKs, which are sequentially activated. MAPK3s directly respond to environmental signals and phosphorylate MAP2Ks, which in turn phosphorylate specific MAPKs. MAPKs then mediated the appropriate cellular response by phosphorylating cellular substrates, including transcription factors that regulate gene expression.

Apoptosis signal-regulating kinase 1 (ASK1) is a member of the mitogen-activated protein kinase kinase kinase (“MAP3K”) family that activates the c-Jun N-terminal protein kinase (“JNK”) and p38 MAP kinase. (Ichijo, H., et al. (1997) Science, 275, 90-94). ASK1 is activated by a variety of stimuli including oxidative stress, reactive oxygen species (ROS), LPS, TNF-α, FasL, ER stress, and increased intracellular calcium concentrations (Hattori, K., et al. (2009) Cell Comm. Signal. 7:1-10; Takeda, K., et al. (2007) Annu. Rev. Pharmacal. Toxicol. 48: 1-8.27; Nagai, H., et al. (2007) J. Biochem. Mol. Biol. 40: 1-6). ASK1 undergoes activation via autophosphorylation at Thr838 in response to these signals and in turn phosphorylates MAP2Ks, such as MKK3/6 and MKK4/7, which then phosphorylate and activate p38 and JNK MAPKs, respectively. ASK2 is a related MAP3K that shares 45% sequence homology with ASK1 (Wang, X. S., et al. (1998) Biochem. Biophys. Res. Commun. 253, 33-37. Although ASK2 tissue distribution is restricted, in some cell types ASK1 and ASK2 have been reported to interact and function together in a protein complex (Takeda, K., et al. (2007) J. Bioi. Chern. 282: 7522-7531; Iriyama, T., et al. (2009) Embo J. 28: 843-853) In non-stressed conditions, A SKI is kept in an inactive state through binding to its repressor Thioredoxin (Trx) (Saitoh, M., et al. (1998) Embo J. 17:2596-2606), and through association with AKT (Zhang, L., Chen, J. and Fu, H. (1999) Proc. Nal Acad. Sci. U.S.A. 96:8511-8515). Phosphorylation of ASK1 protein can lead to apoptosis or 10 other cellular responses depending on the cell type. ASK1 activation and signaling have been reported to play an important role in a broad range of diseases including neurodegenerative, cardiovascular, inflammatory, autoimmunity, and metabolic disorders. In addition, ASK1 has been implicate in mediating organ damage following ischemia and reperfusion of the heart, brain, and kidney (Watanabe et al. (2005) BBRC 333, 562-567; Zhang et al., (2003) Life Sci 74-37-43; Terada et al. (2007) BBRC 364: 1043-49). Emerging evidence suggests that ASK2, either alone or in a complex with ASK1, may play important roles in human diseases as well. Therefore, therapeutic agents that function as inhibitors of ASK1 and ASK2 signaling complexes have the potential to remedy or improve the lives of patients suffering from such conditions. U.S. Publication No. 2007/0276050 describes methods for identifying ASK1 inhibitors useful for preventing and/or treating cardiovascular disease and methods for preventing and/or treating cardiovascular disease in an animal. The methods comprise administering to the animal an ASK1 30 inhibitor and, optionally, a hypertensive compound. U.S. Publication No. 2007/0167386 reports a drug for at least one of prevention and treatment of cardiac failure containing a compound that inhibits a functional expression of ASK1 protein in a cardiomyocyte, and a method for screening the drug. W02009027283 discloses triazolopyridine compounds, methods for preparation thereof and methods for treating autoimmune disorders, inflammatory diseases, cardiovascular diseases and neurodegenerative diseases. U.S. Pat. Nos. 8,552,196 and 8,742,126 teach ASK1 inhibiting compounds useful as pharmaceutical agents.

BET or BRD inhibitors are a class of drugs with anti-cancer, immunosuppressive, and other effects demonstrated in clinical trials and widely used in research. They reversibly bind the bromodomains of Bromodomain and Extra-Terminal motif (BET) proteins BRD2, BRD3, BRD4 and BRDT and prevent protein-protein interaction between BET proteins and acetylated histones and transcription factors. Bromodomain inhibitors include the benzimidazole derivatives taught in US 2014-0336190.

Abnormal activity of certain MMPs plays a role in tumor growth, metastasis, inflammation, autoimmunity, and vascular disease. See, for example, Hu et al. (2007) Nature Reviews: Drug Discovery 6:480-498. One notable source of MMP9 is tumor-associated macrophages (TAMs), which support metastasis and invasion in a complex co-activation loop via paracrine interaction with the primary tumor cells. This combination of the proteolytic breakdown of physical barriers to cell invasion plus liberation of factors that activate growth and angiogenesis paves the way for tumor expansion, with the accompanying development of neovascularization to support tumor outgrowth.

MMP9 is a target of oncogenic signaling pathways such as RAS/RAF, PI3K/AKT/NFkB, and WNT/beta-catenin and functions as an upstream regulator of these pathways via modulation of integrin and receptor tyrosine kinase function. MMP9 is also expressed by subsets of stromal cells (e.g. vasculature, fibroblasts) and tumor-associated infiltrating cells, including myeloid-derived suppressor cells, macrophages and neutrophils. MMP9 is elevated in a wide variety of tumor types and MMP9 levels are correlated with poor prognosis in many cancers, including gastric, lung, and colorectal cancer. MP9 is also implicated in chemoresistance and is upregulated upon loss of several tumor suppressors. MMP9 is upregulated in many diverse tumor types and can promote primary growth and distal invasion of cancerous cells.

It can be desirable to inhibit the activity of one or more MMPs in certain therapeutic settings. However, the activity of certain other MMPs, e.g., MMP2, is often required for normal function and/or is protective against disease. Since most MMP inhibitors are targeted to the conserved catalytic domain and, as a result, inhibit a number of different MMPS, use of available MMP inhibitors has caused side effects due to the inhibition of essential, non-pathogenically-related MMPs. Useful MMP9 inhibitors include the antibodies and fragments disclosed in U.S. 2015-0140580 (Smith et al.) and U.S. Pat. No. 8,377,443 (McAuley et al.), U.S. Pat. No. 8,501,916 (McAuley et al.), and U.S. Pat. No. 9,120,863 (McAuley et al.).

There remains a need for additional treatments for cancers.

### BRIEF SUMMARY

Provided herein are methods for treating cancers, allergic disorders, autoimmune diseases and inflammatory diseases that involve the administration of a BTK inhibitor in combination with one or more inhibitor selected from the group consisting of a JAK inhibitor, a ASK inhibitor, a BRD inhibitor, and a MMP9 inhibitor. In some embodiments, the BTK inhibitor is 6-amino-9-[(3R)-1-(2-butynoyl)-3-pyrrolidinyl]-7-(4-phenoxyphenyl)-7,9-dihydro-8H-purin-8-one, or a pharmaceutically acceptable salt or hydrate thereof. In some variations, the BTK inhibitor is a hydrochloride salt of 6-amino-9-[(3R)-1-(2-butynoyl)-3-pyrrolidinyl]-7-(4-phenoxyphenyl)-7,9-dihydro-8H-purin-8-one, or a pharmaceutically acceptable hydrate thereof.

In some aspects, provided is a method for treating cancer in a human in need thereof, comprising administering to the human a therapeutically effective amount of a BTK inhibitor and a therapeutically effective amount of a JAK inhibitor.

In some embodiments, the JAK inhibitor is selected from the group of momelotinib, peficitinib, tofacitinib, oclacitinib, ruxolitinib, baracitinib, lestaurtinib, pacritinib, filgotinib, 1-[1-[[3-fluoro-2-(trifluoromethyl)-4-pyridinyl]-4-piperidinyl]-3-[4-(7H-pyrrolo[2,3-d]pyrimidin-4-yl)-1H-pyrazol-1-yl]-3-azetidineacetonitrile, TG101348, JS-124, INCB39110, INCB16562, CHZ868, VX-509, XL019, NVP-BSK805, CEP33779, R-348, AC-430, CDP-R723, BMS911543, GSK2586184, or a pharmaceutically acceptable salt or hydrate thereof. In some aspects, provided is a method for treating cancer in a human in need thereof, comprising administering to the human a therapeutically effective amount of a BTK inhibitor and a therapeutically effective amount of a ASK inhibitor. In some embodiments, the ASK inhibitor is selected from the group of Compound C1, Compound C2, or the compound of Formula (I). In some aspects, provided is a method for treating cancer in a human in need thereof, comprising administering to the human a therapeutically effective amount of a BTK inhibitor and a therapeutically effective amount of a BRD inhibitor. In some embodiments, the BRD inhibitor is the compound of Formula (II). In some aspects, provided is a method for treating cancer in a human in need thereof, comprising administering to the human a therapeutically effective amount of a BTK inhibitor and a therapeutically effective amount of an MMP9 inhibitor. In some embodiments, the MMP9 inhibitor is MMP9 binding proteins, e.g., antibodies and antigen-binding fragments thereof, that bind to the matrix metalloproteinase-9 (MMP9) protein (MMP9 is also known as gelatinase-B), wherein the binding proteins comprise an immunoglobulin (Ig) heavy chain (or functional fragment thereof) and an Ig light chain (or functional fragment thereof). In certain embodiments, the MMP9 inhibitor comprises the amino acid sequence selected from the group consisting of SEQ ID NOs: 3, 4, and 5-12. Provided herein are also articles of manufacture and kits that comprise the BTK inhibitor and one or more inhibitor selected from a JAK inhibitor, a ASK inhibitor, a BRD inhibitor, and a MMP9 inhibitor. Also provided herein are methods comprising a BTK inhibitor and one or more inhibitor selected from a JAK inhibitor, a ASK inhibitor, a BRD inhibitor, and a MMP9 inhibitor for the use in therapy or in the manufacture of a medicament for cancer treatment.

### BRIEF DESCRIPTION OF THE DRAWINGS

FIG. 1 provides a chart of Mean±SE Ankle Diameter for a rat collagen-induced arthritis model conducted using Compound A1 and tofacitinib.

FIG. 2: depicts a heat map representing the percent of DLBCL cell growth inhibition for every pairwise combination of Compound A1 and a BET inhibitor 6-amino-9-[(3R)-1-(2-butynoyl)-3-pyrrolidinyl]-7-(4-phenoxyphenyl)-7,9-dihydro-8H-purin-8-one (Compound D) from one representative experiment.

FIG. 3: depicts a heatmap of the calculated Bliss excess over predicted additivity for every pairwise combination using the percent growth inhibition shown in FIG. 2.

FIG. 4: depicts the average percent cell growth inhibition relative to a DMSO control (n=3) for DLBCL cells treated with a dilution series of Compound D either alone or in the presence of 5.5 nM or 11 nM of Compound A1.

### DETAILED DESCRIPTION

The following description sets forth exemplary methods, parameters and the like. It should be recognized, however, that such description is not intended as a limitation on the scope of the present disclosure but is instead provided as a description of exemplary embodiments. Provided are methods, compositions (including pharmaceutical compositions, formulations, or unit dosages), articles of manufacture and kits comprising a BTK inhibitor and one or more inhibitor selected from a JAK inhibitor, a ASK inhibitor, a BRD inhibitor, and a MMP9 inhibitor.

Combinations of pharmaceutically effective amounts of the BTK inhibitor and one or more inhibitor selected from a JAK inhibitor, a ASK inhibitor, a BRD inhibitor, and a MMP9 inhibitor as described herein may be used to treat cancers, allergic disorders, autoimmune diseases and inflammatory diseases in a human, the method comprising administering to the human in need thereof a pharmaceutically effective amount of the BTK inhibitor, or a pharmaceutically acceptable salt or hydrate thereof, and a pharmaceutically effective amount of one or more inhibitor selected from a JAK inhibitor, a ASK inhibitor, a BRD inhibitor, and a MMP9 inhibitor. The combinations taught herein may be used for the treatment of allergic disorders, autoimmune diseases and inflammatory diseases such as: systemic lupus erythematosus (SLE), rheumatoid arthritis (RA), multiple vasculitides, idiopathic thrombocytopenic purpura (ITP), myasthenia gravis, allergic rhinitis, chronic obstructive pulmonary disease (COPD), adult respiratory distress syndrome (ARDs) and asthma. The combinations taught herein may be used for the treatment of cancers such as hematologic malignancy, leukemia, lymphoma chronic lymphocytic leukemia (CLL), small lymphocytic lymphoma (SLL), non-Hodgkin's lymphoma, indolent non-Hodgkin's lymphoma (iNHL), refractory iNHL, mantle cell lymphoma, follicular lymphoma (FL), lymphoplasmacytic lymphoma, and marginal zone lymphoma.

### Definitions

A dash at the front or end of a chemical group is a matter of convenience; chemical groups may be depicted with or without one or more dashes without losing their ordinary meaning. A wavy line drawn through a line in a structure indicates a point of attachment of a group. A dashed line indicates an optional bond. Unless chemically or structurally required, no directionality is indicated or implied by the order in which a chemical group is written. For instance, the group “—SO₂CH₂—” is equivalent to “—CH₂SO₂—” and both may be connected in either direction. The prefix “Cᵤ₋ᵥ” indicates that the following group has from u to v carbon atoms, one or more of which, in certain groups (e.g. heteroalkyl, heteroaryl, heteroarylalkyl, etc.), may be replaced with one or more heteroatoms or heteroatomic groups. For example, “C₁₋₆ alkyl” indicates that the alkyl group has from 1 to 6 carbon atoms.

Also, certain commonly used alternative chemical names may or may not be used. For example, a divalent group such as a divalent “alkyl” group, a divalent “aryl” group, etc., may also be referred to as an “alkylene” group or an “alkylenyl” group, an “arylene” group or an “arylenyl” group, respectively.

“Alkyl” refers to any aliphatic hydrocarbon group, i.e. any linear, branched, cyclic, or spiro nonaromatic hydrocarbon group or an isomer or combination thereof. As used herein, the term “alkyl” includes terms used in the art to describe saturated and unsaturated aliphatic hydrocarbon groups with one or more points of attachment, including alkenyl (an aliphatic group containing at least one carbon-carbon double bond), alkylene (a divalent aliphatic group), alkynyl (an aliphatic group containing at least one carbon-carbon triple bond), cycloalkyl (a cyclic aliphatic group), alkylcycloalkyl (a linear or branched aliphatic group attached to a cyclic aliphatic group), and the like. Alkyl groups include, but are not limited to, methyl; ethyl; propyls such as propan-1-yl, propan-2-yl (iso-propyl), and cyclopropyls such as cyclopropan-1-yl, etc.; butyls such as butan-1-yl, butan-2-yl (sec-butyl), 2-methyl-propan-1-yl (iso-butyl), 2-methyl-propan-2-yl (t-butyl), cyclobutan-1-yl; butenes (e.g. (E)-but-2-ene, (Z)-but-2-ene); pentyls; pentenes; hexyls; hexenes; octyls; decyls; cyclopropyl, cyclobutyl, cyclopentyl, cyclohexyl, methylcyclohexyl, spiro[2.4]heptyl, and the like. An alkyl group comprises from 1 to about 10 carbon atoms, e.g., from 1 to 6 carbon atoms. In some embodiments, alkyl is a monovalent, linear or branched, saturated aliphatic hydrocarbon group comprising from 1 to about 10 carbon atoms, e.g., from 1 to 6 carbon atoms.

“Alkenyl” is a subset of“alkyl” and refers to an aliphatic group containing at least one carbon-carbon double bond and having from 2 to about 10 carbon atoms, e.g., from 2 to 6 carbon atoms or 2 to 4 carbon atoms and having at least one site of vinyl unsaturation (>C═C<). Alkenyl groups include ethenyl, propenyl, 1,3-butadienyl, and the like. Alkynyl may have from 2 to about 10 carbon atoms, e.g. from 2 to 6 carbon atoms or 2 to 4 carbon atoms.

“Alkynyl” is a subset of “alkyl” and refers to an aliphatic group containing at least one carbon-carbon triple bond. The term “alkynyl” is also meant to include those groups having one triple bond and one double bond.

“Alkoxy” refers to the group —O-alkyl, wherein the alkyl group may be optionally substituted. Alkoxy includes, by way of example, methoxy, ethoxy, n-propoxy, isopropoxy, n-butoxy, t-butoxy, sec-butoxy, and n-pentoxy.

“Acyl” refers to a group —C(═O)R, where R is hydrogen, alkyl, cycloalkyl, cycloheteroalkyl, aryl, arylalkyl, heteroalkyl, heteroaryl or heteroarylalkyl as defined herein, each of which may be optionally substituted, as defined herein. Representative examples include, but are not limited to formyl, acetyl, cylcohexylcarbonyl, cyclohexylmethyl-carbonyl, benzoyl, benzyloxycarbonyl and the like.

“Amido” refers to both a “C-amido” group which refers to the group —C(═O)NR^{y}R^{z} and an “N-amido” group which refers to the group —NR^{y}C(═O)R^{z}, wherein R^{y} and R^{z} are independently selected from the group consisting of hydrogen, alkyl, aryl, heteroalkyl, heteroaryl (each of which may be optionally substituted), and where R^{y} and R^{z} are optionally joined together with the nitrogen or carbon bound thereto to form an optionally substituted heterocycloalkyl.

“Amino” refers to the group —NR^{y}R^{z} wherein R^{y} and R^{z} are independently selected from the group consisting of hydrogen, alkyl, aryl, heteroalkyl, heteroaryl (each of which may be optionally substituted), and where R^{y} and R^{z} are optionally joined together with the nitrogen bound thereto to form a heterocycloalkyl or heteroaryl heteroaryl (each of which may be optionally substituted).

“Amidino” refers to the group —C(═NR^{x})NR^{y}R^{z} where R^{x}, R^{y}, and R^{z} are independently selected from the group consisting of hydrogen, alkyl, aryl, heteroalkyl, heteroaryl (each of which may be optionally substituted), and where R^{y} and R^{z} are optionally joined together with the nitrogen bound thereto to form a heterocycloalkyl or heteroaryl (each of which may be optionally substituted).

“Aryl” refers to a group with one or more aromatic rings. It may be a single aromatic ring or multiple aromatic rings which are fused together, linked covalently, or linked via one or more such as a methylene or ethylene moiety. Aryl groups include, but are not limited to, those groups derived from acenaphthylene, anthracene, azulene, benzene, biphenyl, chrysene, cyclopentadienyl anion, diphenylmethyl, fluoranthene, fluorene, indane, indene, naphthalene, perylene, phenalene, phenanthrene, pyrene, triphenylene, and the like. An aryl group comprises from 5 to about 20 carbon atoms, e.g., from 5 to 20 carbon atoms, e.g. from 5 to 10 carbon atoms. In some embodiments, aryl is a a single aromatic ring or multiple aromatic rings which are fused together.

“Arylalkyl” (also “aralkyl”) refers to an aryl group attached to an alkyl group. Arylalkyl groups include, but are not limited to, benzyl, tolyl, dimethylphenyl, 2-phenylethan-1-yl, 2-naphthylmethyl, 2-naphthylethan-1-yl, naphthobenzyl, phenylvinyl, diphenylmethyl, and the like. For example, the “arylalkyl” may be attached to the rest of the compound of formula (I) through the aryl group. Alternatively, the “arylalkyl” may be attached to the rest of the compound of formula (I) through the alkyl group. Where specific alkyl moieties are intended, the nomenclature arylalkanyl, arylalkenyl and/or arylalkynyl may be used. An arylalkyl group comprises from 6 to about 30 carbon atoms, e.g. the alkyl portion of the arylalkyl group can comprise from 1 to about 10 carbon atoms and the aryl portion of the arylalkyl group can comprise from 5 to about 20 carbon atoms. In some instances an arylalkyl group comprises from 6 to about 20 carbon atoms, e.g. the alkyl portion of the arylalkyl group can comprise from 1 to about 10 carbon atoms and the aryl portion of the arylalkyl group can comprise from 5 to about 10 carbon atoms.

“Aryloxy” refers to the group —O-aryl, including by way of example, phenoxy and naphthoxy.

“Azido” refers to the group —N₃.

“Boronic acid” refers to the group —B(OH)₂.

Boronic acid ester” refers to an ester derivative of a boronic acid compound. Suitable boronic acid ester derivatives include those of the formula —B(OR)₂ where R is hydrogen, alkyl, aryl, arylalkyl, heteroalkyl, or heteroaryl, each of which may be optionally substituted. For example, boronic acid ester may be pinacol ester or catechol ester.

“Carbocycle” or “carbocyclyl” refers to a saturated, partially unsaturated or aromatic ring having 3 to 7 carbon atoms as a monocycle, 7 to 12 carbon atoms as a bicycle, and up to about 20 carbon atoms as a polycycle. Monocyclic carbocycles have 3 to 6 ring atoms, still more typically 5 or 6 ring atoms. Bicyclic carbocycles have 7 to 12 ring atoms, e.g., arranged as a bicyclo (4,5), (5,5), (5,6) or (6,6) system, or 9 or 10 ring atoms arranged as a bicyclo (5,6) or (6,6) system. Carbocycles includes aromatic and non-aromatic mono-, bi-, and poly-cyclic rings, whether fused, bridged, or spiro. Non-limiting examples of monocyclic carbocycles include the cycloalkyls group such as cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl or aryl groups such as phenyl, and the like. Thus, “carbocycle,” as used herein, encompasses but is not limited to “aryl”, “phenyl” and “biphenyl.”

“Carbamoyl” refers to the group —C(O)NR^{y}R^{z} where R^{y} and R^{z} are defined as in “amino” above.

“Carbonyl” refers to the divalent group —C(O)— which is equivalent to —C(═O)—.

“Carboxyl” or “carboxy” refers to —COOH or salts thereof.

“Carboxyl ester” or “carboxy ester” refers to the groups —C(O)OR, wherein R is hydrogen, alkyl, aryl, arylalkyl, heteroalkyl, or heteroaryl, each of which may be optionally substituted. In one embodiment, R is alkyl, aryl, arylalkyl, heteroalkyl, or heteroaryl, each of which may be optionally substituted.

“Cyano” or “carbonitrile” refers to the group —CN.

“Cycloalkyl” is a subset of “alkyl” and refers to a saturated or partially saturated cyclic group of from 3 to about 10 carbon atoms and no ring heteroatoms and having a single ring or multiple rings including fused, bridged, and spiro ring systems. For multiple ring systems having aromatic and non-aromatic rings that have no ring heteroatoms, the term “cycloalkyl” applies when the point of attachment is at a non-aromatic carbon atom (e.g., 5,6,7,8,-tetrahydronaphthalene-5-yl). The term “cycloalkyl” includes cycloalkenyl groups. Examples of cycloalkyl groups include, for instance, adamantyl, cyclopropyl, cyclobutyl, cyclopentyl, cyclooctyl, and cyclohexenyl.

“Guanidino” refers to the group —NHC(═NH)NHz.

“Halo” or “halogen” refers to fluoro, chloro, bromo and iodo.

“Haloalkyl” refers to substitution of alkyl groups with 1 to 5 or, in some embodiments, 1 to 3 halo groups, e.g., —CH₂Cl, —CH₂F, —CH₂Br, —CFClBr, —CH₂CH₂Cl, —CH₂CH₂F, —CF₃, —CH₂CF₃, —CH₂CCl₃, and the like, and further includes those alkyl groups such as perfluoroalkyl in which all hydrogen atoms are replaced by fluorine atoms.

“Haloaryl” refers to aryl groups with one or more halo or halogen substituents. For example, haloaryl groups include phenyl groups in which from 1 to 5 hydrogens are replaced with a halogen. Haloaryl groups include, for example, fluorophenyl, difluorophenyl, trifluorophenyl, chlorophenyl, chlorofluorophenyl, and the like.

“Heteroalkyl” refers to an alkyl group in which one or more of the carbon atoms (and any associated hydrogen atoms) are each independently replaced with the same or different heteroatom or heteroatomic group. For example, heteroalkyl may include 1, 2 or 3 heteroatomic groups, e.g. 1 heteroatomic group. Heteroatoms include, but are not limited to, N, P, O, S, etc. Heteroatomic groups include, but are not limited to, —NR—, —O—, —S—, —PH—, —P(O)₂—, —S(O)—, —S(O)₂—, and the like, where R is H, alkyl, aryl, cycloalkyl, heteroalkyl, heteroaryl or cycloheteroalkyl. The term “heteroalkyl” includes heterocycloalkyl (a cyclic heteroalkyl group), alkyl-heterocycloalkyl (a linear or branched aliphatic group attached to a cyclic heteroalkyl group), and the like. Heteroalkyl groups include, but are not limited to, —OCH₃, —CH₂OCH₃, —SCH₃, —CH₂SCH₃, —NRCH₃, —CH₂NRCH₃, and the like, where R is hydrogen, alkyl, aryl, arylalkyl, heteroalkyl, or heteroaryl, each of which may be optionally substituted. A heteroalkyl group comprises from 1 to about 10 carbon and hetero atoms, e.g., from 1 to 6 carbon and hetero atoms.

“Heteroaryl” refers to an aryl group in which one or more of the carbon atoms (and any associated hydrogen atoms) are each independently replaced with the same or different heteroatoms, as defined above. For example, heteroaryl may include 1, 2 or 3 heteroatomic groups, e.g. 1 heteroatomic group. Heteroaryl groups include, but are not limited to, groups derived from acridine, benzoimidazole, benzothiophene, benzofuran, benzoxazole, benzothiazole, carbazole, carboline, cinnoline, furan, imidazole, imidazopyridine, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. A heteroaryl group comprises from 5 to about 20 carbon and hetero atoms in the ring or rings, e.g., from 5 to 20 carbon and hetero atoms, e.g. from 5 to 10 carbon and hetero atoms.

“Heteroarylalkyl” refers to an arylalkyl group in which one or more carbon atoms (and any associated hydrogen atoms) are independently replaced with the same or different heteroatoms, as defined above. For example, heteroarylalkyl may include 1, 2 or 3 heteroatomic groups. Heteroarylalkyl groups include, but are no limited to, groups derived from heteroaryl groups with alkyl substituents (e.g. methylpyridine, dimethylisoxazole, etc.), hydrogenated heteroaryl groups (dihydroquinolines, e.g. 3,4-dihydroquinoline, dihydroisoquinolines, e.g. 1,2-dihydroisoquinoline, dihydroimidazole, tetrahydroimidazole, etc.), isoindoline, isoindolones (e.g. isoindolin-1-one), dihydrophthalazine, quinolinone, spiro[cyclopropane-1,1′-isoindolin]-3′-one, di(pyridin-2-yl)methyl, di(pyridin-3-yl)methyl, di(pyridin-4-yl)methyl, and the like. A heteroarylalkyl group comprises from 6 to about 30 carbon and hetero atoms, for example from 6 to about 20 carbon and hetero atoms.

“Heterocycloalkyl” is a subset of “heteroalkyl” and refers to a saturated or unsaturated cycloalkyl group in which one or more carbon atoms (and any associated hydrogen atoms) are independently replaced with the same or different heteroatom. Heteroatoms include, but are not limited to, N, P, O, S, etc. A heterocycloalkyl group may also contain a charged heteroatom or group, e.g., a quaternized ammonium group such as —N+(R)2- wherein R is alkyl, e.g., methyl, ethyl, etc. Heterocycloalkyl groups include, but are not limited to, groups derived from epoxide, imidazolidine, morpholine, piperazine, piperidine, pyrazolidine, piperidine, pyrrolidine, pyrrolidinone, tetrahydrofuran, tetrahydrothiophene, dihydropyridine, tetrahydropyridine, quinuclidine, N-bromopyrrolidine, N-bromopiperidine, N-chloropyrrolidine, N-chloropiperidine, an N,N-dialkylpyrrolidinium, such as N,N-dimethylpyrrolidinium, a N,N-dialkylpiperidinium such as N,N-dimethylpiperidium, and the like. The heterocycloalkyl group comprises from 3 to about 10 carbon and hetero atoms in the ring or rings. In some embodiments, heterocycloalkyl includes 1, 2 or 3 heteroatomic groups.

“Heterocycle” or “heterocyclyl” as used herein includes by way of example and not limitation those heterocycles described in Paquette, Leo A.; Principles of Modern Heterocyclic Chemistry (W. A. Benjamin, New York, 1968), particularly Chapters 1, 3, 4, 6, 7, and 9; The Chemistry of Heterocyclic Compounds. A Series of Monographs” (John Wiley & Sons, New York, 1950 to present), in particular Volumes 13, 14, 16, 19, and 28; and J. Am. Chem. Soc. (1960) 82:5566. In one specific embodiment of the invention “heterocycle” includes a “carbocycle” as defined herein, wherein one or more (e.g. 1, 2, 3, or 4) carbon atoms have been replaced with a heteroatom (e.g. O, N, P or S). The terms “heterocycle” or “heterocyclyl” includes saturated rings, partially unsaturated rings, and aromatic rings (i.e., heteroaromatic rings). Heterocycles includes aromatic and non-aromatic mono-, bi-, and poly-cyclic rings, whether fused, bridged, or spiro. As used herein, the term “heterocycle” encompasses, but is not limited to “heteroaryl.” Substituted heterocyclyls include, for example, heterocyclic rings substituted with any of the substituents disclosed herein including carbonyl groups. Examples of heterocycles include by way of example and not limitation pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, azetidinyl, 2-pyrrolidonyl, pyrrolinyl, tetrahydrofuranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, isatinoyl, and bis-tetrahydrofuranyl.

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl. By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

“Hydrazino” refers to the group —NHNH₂.

“Hydroxy” or “hydroxyl” refers to the group —OH.

“Imino” refers to the group —C(═NR)— wherein R is hydrogen, alkyl, aryl, arylalkyl, heteroalkyl, or heteroaryl, each of which may be optionally substituted.

“Nitro” refers to the group —NO₂.

The terms “optional” or “optionally” mean that the subsequently described event or circumstance may but need not occur, and that the description includes instances where the event or circumstance occurs and instances in which it does not.

“Oxide” refers to products resulting from the oxidation of one or more heteroatoms. Examples include N-oxides, sulfoxides, and sulfones.

“Oxo” refers to a double-bonded oxygen (═O). In compounds where an oxo group is bound to an sp² nitrogen atom, an N-oxide is indicated.

“Racemates” refers to a mixture of enantiomers.

“Stereoisomer” or “stereoisomers” refer to compounds that differ in the chirality of one or more stereocenters. Stereoisomers include enantiomers and diastereomers. The compounds may exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of Advanced Organic Chemistry, 4th ed., J. March, John Wiley and Sons, New York, 1992).

“Substituted” (as in, e.g., “substituted alkyl”) refers to a group wherein one or more hydrogens have been independently replaced with one or more substituents including, but not limited to, alkyl, alkenyl, alkynyl, alkoxy, acyl, amino, amido, amidino, aryl, azido, carbamoyl, carboxyl, carboxyl ester, cyano, guanidino, halo, haloalkyl, heteroalkyl, heteroaryl, heterocycloalkyl, hydroxy, hydrazino, hydroxyl, imino, oxo, nitro, sulfinyl, sulfonic acid, sulfonyl, thiocyanate, thiol, thione, or combinations thereof. Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to -substituted aryl-(substituted aryl)-substituted aryl. For example, in some embodiments, when a group described above as being “optionally substituted” is substituted, that substituent is itself unsubstituted. Similarly, it is understood that the above definitions are not intended to include impermissible substitution patterns (e.g., methyl substituted with 5 fluoro groups or heteroaryl groups having two adjacent oxygen ring atoms). Such impermissible substitution patterns are well known to the skilled artisan. When used to modify a chemical group, the term “substituted” may describe other chemical groups defined herein. For example, the term “substituted aryl” includes, but is not limited to, “arylalkyl.” Generally, substituted groups will have 1 to 5 substituents, 1 to 3 substituents, 1 or 2 substituents or 1 substituent. Alternatively, the optionally substituted groups of the invention may be unsubstituted.

“Sulfonyl” refers to the divalent group —S(O)₂—.

“Tautomer” refers to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Thiocyanate” refers to the group —SCN.

“Thiol” refers to the group —SH.

Thione” refers to a thioketone (═S) group.

“Pharmaceutically acceptable” refers to compounds, salts, compositions, dosage forms and other materials which are useful in preparing a pharmaceutical composition that is suitable for veterinary or human pharmaceutical use.

“Pharmaceutically acceptable salt” refers to a salt of a compound that is pharmaceutically acceptable and that possesses (or can be converted to a form that possesses) the desired pharmacological activity of the parent compound. Such salts include acid addition salts formed with inorganic acids such as hydrochloric acid, hydrobromic acid, sulfuric acid, nitric acid, phosphoric acid, and the like; or formed with organic acids such as acetic acid, benzenesulfonic acid, benzoic acid, camphorsulfonic acid, citric acid, ethanesulfonic acid, fumaric acid, glucoheptonic acid, gluconic acid, lactic acid, maleic acid, malonic acid, mandelic acid, methanesulfonic acid, 2-napththalenesulfonic acid, oleic acid, palmitic acid, propionic acid, stearic acid, succinic acid, tartaric acid, p-toluenesulfonic acid, trimethylacetic acid, and the like, and salts formed when an acidic proton present in the parent compound is replaced by either a metal ion, e.g., an alkali metal ion, an alkaline earth ion, or an aluminum ion; or coordinates with an organic base such as diethanolamine, triethanolamine, N-methylglucamine and the like. Also included in this definition are ammonium and substituted or quaternized ammonium salts. Representative non-limiting lists of pharmaceutically acceptable salts can be found in S. M. Berge et al., J. Pharma Sci., 66(1), 1-19 (1977), and Remington: The Science and Practice of Pharmacy, R. Hendrickson, ed., 21st edition, Lippincott, Williams & Wilkins, Philadelphia, Pa., (2005), at p. 732, Table 38-5, both of which are hereby incorporated by reference herein.

The following abbreviations may also be used: AcOH: acetic acid; nBuLi: n-butyllithium; CC: column chromatography; Cs₂CO₃: cesium carbonate; CH₂Cl₂ or DCM: dichloromethane; CH₃MgI: methyl magnesium iodide; CuCl₂: copper chloride; DAST: (diethylamino)sulfur trifluoride; DEAD: diethyl azodicarboxylate; DIBAL: diisobutylaluminum hydride; DIPEA: diisopropylethylamine; DMF: dimethylformamide; DMSO: dimethyl sulfoxide; Et₃N: triethylamine; EtOAc: ethyl acetate; EtOH: ethanol; g: gram(s); h: hour; H₂: hydrogen; HBr: hydrogen bromide; HCl: hydrogen chloride; H₂O: water; H₂O₂: hydrogen peroxide; HPLC: high performance liquid chromatography; KCN: potassium cyanide; LHMDS: lithium hexamethyldisilazide; LiAlH₄: lithium aluminum hydride; LiOH: lithium hydroxide; M: molar, MeCN: acetonitrile; MeI: methyl iodide; MeOH: methanol; MgSO₄: magnesium sulfate; MgCO₃: magnesium carbonate; mg: milligram; MsCl: mesyl chloride; mmol: millimoles mL: milliliter, sodium hydrogen sulfite; mCPBA: meta-chloroperoxybenzoic acid; N: normality; N₂: nitrogen; Na₂CO₃: sodium carbonate; NaHCO₃: sodium bicarbonate; NaNO₂: sodium nitrite; NaOH: sodium hydroxide; Na₂S₂O₃: sodium bisulfate; Na₂SO₄: sodium sulfate; NBS: N-bromosuccinimide; NH₄Cl: ammonium chloride; NH₄OAc: ammonium acetate; NMR: nuclear magnetic resonance; Pd/C: palladium on carbon; PPh₃: triphenyl phosphine; iPrOH: isopropyl alcohol; RT: room temperature; SOCl₂: thionyl chloride; THF: tetrahydrofuran; TLC: thin layer chromatography; μL: microliter.

It is understood that combinations of chemical groups may be used and will be recognized by persons of ordinary skill in the art. For instance, the group “hydroxyalkyl” would refer to a hydroxyl group attached to an alkyl group. A great number of such combinations may be readily envisaged.

Compounds of a given formula described herein encompasses the compound disclosed and all pharmaceutically acceptable salts, esters, stereoisomers, tautomers, prodrugs, solvates, and deuterated forms thereof, unless otherwise specified.

“Effective amount” or “therapeutically effective amount” means the amount of a compound or molecule described herein that may be effective to elicit the desired biological or medical response. These terms include the amount of a compound that, when administered to a subject for treating a disease, is sufficient to effect such treatment for the disease. The effective amount will vary depending on the compound, the disease and its severity and the age, weight, etc., of the subject to be treated.

In another aspect, provided herein is a method for treating a human who is “refractory” to a cancer treatment or who is in “relapse” after treatment for cancer (e.g., a hematologic malignancy). A subject “refractory” to an anti-cancer therapy means they do not respond to the particular treatment, also referred to as resistant. The cancer may be resistant to treatment from the beginning of treatment, or may become resistant during the course of treatment, for example after the treatment has shown some effect on the cancer, but not enough to be considered a remission or partial remission. A subject in “relapse” means that the cancer has returned or the signs and symptoms of cancer have returned after a period of improvement, e.g. after a treatment has shown effective reduction in the cancer, such as after a subject is in remission or partial remission.

In some variations, the human is (i) refractory to at least one anti-cancer therapy, or (ii) in relapse after treatment with at least one anti-cancer therapy, or both (i) and (ii). In some of embodiments, the human is refractory to at least two, at least three, or at least four anti-cancer therapies (including, for example, standard or experimental chemotherapies).

“Subject” and “subjects” refer to human in need thereof may be an individual who has or is suspected of having a cancer. In some of variations, the human is at risk of developing a cancer (e.g., a human who is genetically or otherwise predisposed to developing a cancer) and who has or has not been diagnosed with the cancer. As used herein, an “at risk” subject is a subject who is at risk of developing cancer (e.g., a hematologic malignancy). The subject may or may not have detectable disease, and may or may not have displayed detectable disease prior to the treatment methods described herein. An at risk subject may have one or more so-called risk factors, which are measurable parameters that correlate with development of cancer, such as described herein. A subject having one or more of these risk factors has a higher probability of developing cancer than an individual without these risk factor(s). These risk factors may include, for example, age, sex, race, diet, history of previous disease, presence of precursor disease, genetic (e.g., hereditary) considerations, and environmental exposure. In some embodiments, a human at risk for cancer includes, for example, a human whose relatives have experienced this disease, and those whose risk is determined by analysis of genetic or biochemical markers. Prior history of having cancer may also be a risk factor for instances of cancer recurrence

As used herein, “treatment” or “treating” is an approach for obtaining beneficial or desired results including clinical results. Beneficial or desired clinical results may include one or more of the following:

(i) inhibiting the disease or condition (e.g., decreasing one or more symptoms resulting from the disease or condition, and/or diminishing the extent of the disease or condition);
(ii) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or
(iii) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival).

In some variations, “delaying” the development of a disease or condition means to defer, hinder, slow, retard, stabilize, and/or postpone development of the disease or condition. This delay can be of varying lengths of time, depending on the history of the disease or condition, and/or subject being treated. For example, a method that “delays” development of a disease or condition is a method that reduces probability of disease or condition development in a given time frame and/or reduces the extent of the disease or condition in a given time frame, when compared to not using the method. Such comparisons are typically based on clinical studies, using a statistically significant number of subjects. Disease or condition development can be detectable using standard methods, such as routine physical exams, mammography, imaging, or biopsy. Development may also refer to disease or condition progression that may be initially undetectable and includes occurrence, recurrence, and onset.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

### Antibodies

As used herein, the term “antibody” means an isolated or recombinant polypeptide binding agent that comprises peptide sequences (e.g., variable region sequences) that specifically bind an antigenic epitope. The term is used in its broadest sense and specifically covers monoclonal antibodies (including full-length monoclonal antibodies), polyclonal antibodies, human antibodies, humanized antibodies, chimeric antibodies, nanobodies, diabodies, multispecific antibodies (e.g., bispecific antibodies), and antibody fragments including but not limited to Fv, scFv, Fab, Fab′ F(ab′)₂ and Fab₂, so long as they exhibit the desired biological activity. The term “human antibody” refers to antibodies containing sequences of human origin, except for possible non-human CDR regions, and does not imply that the full structure of an immunoglobulin molecule be present, only that the antibody has minimal immunogenic effect in a human (i.e., does not induce the production of antibodies to itself).

An “antibody fragment” comprises a portion of a full-length antibody, for example, the antigen binding or variable region of a full-length antibody. Such antibody fragments may also be referred to herein as “functional fragments: or “antigen-binding fragments”. Examples of antibody fragments include Fab, Fab′, F(ab′)₂, and Fv fragments; diabodies; linear antibodies (Zapata et al. (1995) Protein Eng. 8(10):1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three complementarity-determining regions (CDRs) of each variable domain interact to define an antigen-binding site on the surface of the V_{H}-V_{L} dimer. Collectively, the six CDRs confer antigen-binding specificity to the antibody. However, even a single variable domain (or an isolated V_{H} or V_{L} region comprising only three of the six CDRs specific for an antigen) has the ability to recognize and bind antigen, although generally at a lower affinity than does the entire F, fragment.

The “F_{b},” fragment also contains, in addition to heavy and light chain variable regions, the constant domain of the light chain and the first constant domain (CH₁) of the heavy chain. Fab fragments were originally observed following papain digestion of an antibody. Fab′ fragments differ from Fab fragments in that F(ab′) fragments contain several additional residues at the carboxy terminus of the heavy chain CH₁ domain, including one or more cysteines from the antibody hinge region. F(ab′)₂ fragments contain two Fab fragments joined, near the hinge region, by disulfide bonds, and were originally observed following pepsin digestion of an antibody. Fab′-SH is the designation herein for Fab′ fragments in which the cysteine residue(s) of the constant domains bear a free thiol group. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to five major classes: IgA, IgD, IgE, IgG, and IgM, and several of these may be further divided into subclasses (isotypes), e.g., IgG1, IgG2, IgG3, IgG4, IgA1, and IgA2.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun, in The Pharmacology of Monoclonal Antibodies, vol. 113 (Rosenburg and Moore eds.) Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain, thereby creating two antigen-binding sites. Diabodies are additionally described, for example, in EP 404,097; WO 93/11161 and Hollinger et al. (1993) Proc. Natl. Acad. Sci. USA 90:6444-6448.

An “isolated” antibody is one that has been identified and separated and/or recovered from a component of its natural environment. Components of its natural environment may include enzymes, hormones, and other proteinaceous or nonproteinaceous solutes. In some embodiments, an isolated antibody is purified (1) to greater than 95% by weight of antibody as determined by the Lowry method, for example, more than 99% by weight, (2) to a degree sufficient to obtain at least 15 residues of N-terminal or internal amino acid sequence, e.g., by use of a spinning cup sequenator, or (3) to homogeneity by gel electrophoresis (e.g., SDS-PAGE) under reducing or nonreducing conditions, with detection by Coomassie blue or silver stain. The term “isolated antibody” includes an antibody in situ within recombinant cells, since at least one component of the antibody's natural environment will not be present. In certain embodiments, isolated antibody is prepared by at least one purification step.

As used herein, “immunoreactive” refers to antibodies or fragments thereof that are specific to a sequence of amino acid residues (“binding site” or “epitope”), yet if are cross-reactive to other peptides/proteins, are not toxic at the levels at which they are formulated for administration to human use. “Epitope” refers to that portion of an antigen capable of forming a binding interaction with an antibody or antigen binding fragment thereof. An epitope can be a linear peptide sequence (i.e., “continuous”) or can be composed of noncontiguous amino acid sequences (i.e., “conformational” or “discontinuous”). The term “preferentially binds” means that the binding agent binds to the binding site with greater affinity than it binds unrelated amino acid sequences.

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison.

TABLE 1
CDR Definitions

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

As used herein, the term “framework” when used in reference to an antibody variable region is intended to mean all amino acid residues outside the CDR regions within the variable region of an antibody. A variable region framework is generally a discontinuous amino acid sequence between about 100-120 amino acids in length but is intended to reference only those amino acids outside of the CDRs. As used herein, the term “framework region” is intended to mean each domain of the framework that is separated by the CDRs.

“Homology” or “identity” or “similarity” as used herein in the context of nucleic acids and polypeptides refers to the relationship between two polypeptides or two nucleic acid molecules based on an alignment of the amino acid sequences or nucleic acid sequences, respectively. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. In comparing two sequences, the absence of residues (amino acids or nucleic acids) or presence of extra residues also decreases the identity and homology/similarity.

As used herein, “identity” means the percentage of identical nucleotide or amino acid residues at corresponding positions in two or more sequences when the sequences are aligned to maximize sequence matching, i.e., taking into account gaps and insertions. Sequences are generally aligned for maximum correspondence over a designated region, e.g., a region at least about 20, 25, 30, 35, 40, 45, 50, 55, 60, 65 or more amino acids or nucleotides in length, and can be up to the full-length of the reference amino acid or nucleotide. For sequence comparison, typically one sequence acts as a reference sequence, to which test sequences are compared. When using a sequence comparison algorithm, test and reference sequences are input into a computer program, subsequence coordinates are designated, if necessary, and sequence algorithm program parameters are designated. The sequence comparison algorithm then calculates the percent sequence identity for the test sequence(s) relative to the reference sequence, based on the designated program parameters.

Examples of algorithms that are suitable for determining percent sequence identity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (www.ncbi.nlm.nih.gov). Further exemplary algorithms include ClustalW (Higgins D., et al. (1994) Nucleic Acids Res 22: 4673-4680), available at www.ebi.ac.uk/Tools/clustalw/index.html.

Residue positions which are not identical can differ by conservative amino acid substitutions. Conservative amino acid substitutions refer to the interchangeability of residues having similar side chains. For example, a group of amino acids having aliphatic side chains is glycine, alanine, valine, leucine, and isolcucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine.

### Compounds

The compound names provided herein are named using ChemBioDraw Ultra. One skilled in the art understands that the compound may be named or identified using various commonly recognized nomenclature systems and symbols. By way of example, the compound may be named or identified with common names, systematic or non-systematic names. The nomenclature systems and symbols that are commonly recognized in the art of chemistry include, for example, Chemical Abstract Service (CAS), ChemBioDraw Ultra, and International Union of Pure and Applied Chemistry (IUPAC).

Also provided herein are isotopically labeled forms of compounds detailed herein. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H, ¹³C and ¹⁴C are incorporated, are provided. Such isotopically labeled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of subjects (e.g. humans). Also provided for isotopically labeled compounds described herein are any pharmaceutically acceptable salts, or hydrates, as the case may be.

In some variations, the compounds disclosed herein may be varied such that from 1 to n hydrogens attached to a carbon atom is/are replaced by deuterium, in which n is the number of hydrogens in the molecule. Such compounds may exhibit increased resistance to metabolism and are thus useful for increasing the half life of the compound when administered to a mammal. See, for example, Foster, “Deuterium Isotope Effects in Studies of Drug Metabolism”, Trends Pharmacol. Sci. 5(12):524-527 (1984). Such compounds are synthesized by means well known in the art, for example by employing starting materials in which one or more hydrogens have been replaced by deuterium.

Deuterium labeled or substituted therapeutic compounds of the disclosure may have improved DMPK (drug metabolism and pharmacokinetics) properties, relating to absorption, distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compounds provided herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

### BTK Inhibitor

In some variations, the BTK inhibitor is Compound A1, or a pharmaceutically acceptable salt or hydrate thereof. Compound A1 has the structure:

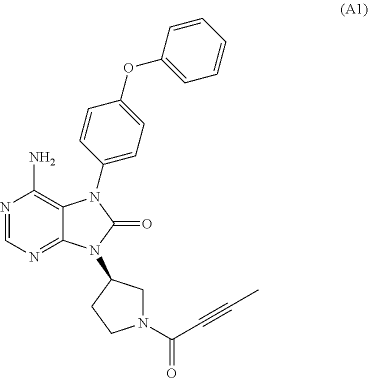 [SMILES: CC#CC(=O)N1CC[C@@H](n2c(=O)n(-c3ccc(Oc4ccccc4)cc3)c3c(N)ncnc32)C1]

In some variations, the BTK inhibitor is a hydrochloride salt of Compound A1, or a hydrate thereof. Compound A1 may be synthesized according to the methods described in U.S. Pat. No. 8,557,803 (Yamamoto et al.) and US 2014/0330015. Compound A1 may be referred to as (R)-6-amino-9-(1-(but-2-ynoyl)pyrrolidin-3-yl)-7-(4-phenoxyphenyl)-7H-purin-8(9H)-one or 6-amino-9-[(3R)-1-(2-butynoyl)-3-pyrrolidinyl]-7-(4-phenoxyphenyl)-7,9-dihydro-8H-purin-8-one. Additional BTK inhibitors include, but are not limited to, (S)-6-amino-9-(1-(but-2-ynoyl)pyrrolidin-3-yl)-7-(4-phenoxyphenyl)-7H-purin-8(9H)-one ibrutinib (1-[(3R)-3-[4-Amino-3-(4-phenoxyphenyl)-1H-pyrazolo[3,4-d]pyrimidin-1-yl]piperidin-1-yl]prop-2-en-1-one), acalabrutinib, HM71224, CNX-774, RN486, ONO-4059, and CC-292 (speburtinib).

### JAK Inhibitor

In some variations, the JAK inhibitor is Compound B1, Compound B2, Compound B3, or Compound B4, or a pharmaceutically acceptable salt thereof. Compound B1, which may be referred to as momelotinib, CYT1137, CYT387, or N-(cyanomethyl)-4-[2-[[4-(4-morpholinyl)phenyl]amino]-4-pyrimidinyl]-benzamide or N-(cyanomethyl)-4-(2-((4-morpholinophenyl) amino)pyrimidin-4-yl)benzamide, has the structure:

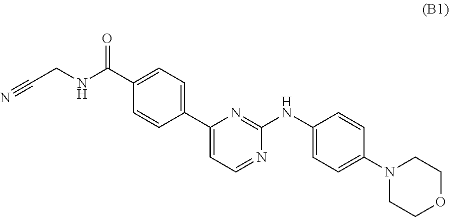 [SMILES: N#CCNC(=O)c1ccc(-c2ccnc(Nc3ccc(N4CCOCC4)cc3)n2)cc1]

Compound B2, which may be referred to as filgotinib, GLPG0634, G146034, N-(5-(4-((1,1-dioxidothiomorpholino)methyl)phenyl)-[1,2,4]triazolo[1,5-a]pyridin-2-yl)cyclopropanecarboxamide, N-[5-[4-[(1,1-dioxo-1,4-thiazinan-4-yl)methyl]phenyl]-[1,2,4]triazolo[1,5-a]pyridin-2-yl]cyclopropanecarboxamide, or N-[5-[4-[(1,1-dioxido-4-thiomorpholinyl) methyl]phenyl][1,2,4]triazolo[1,5-a]pyridin-2-yl]-cyclopropanecarboxamide and has the structure:

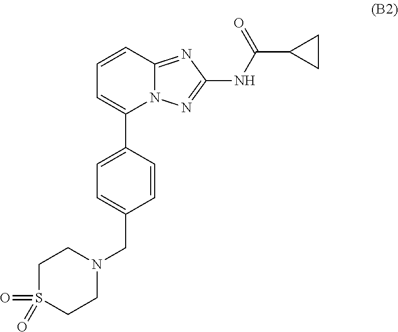 [SMILES: O=C(Nc1nc2cccc(-c3ccc(CN4CCS(=O)(=O)CC4)cc3)n2n1)C1CC1]

Compound B3, which has the Chemical Abstracts registry number 1334298-90-6, may be referred to as 1-[1-[[3-fluoro-2-(trifluoromethyl)-4-pyridinyl]carbonyl]-4-piperidinyl]-3-[4-(7H-pyrrolo[2,3-d]pyrimidin-4-yl)-1H-pyrazol-1-yl]-3-azetidineacetonitrile and has the structure:

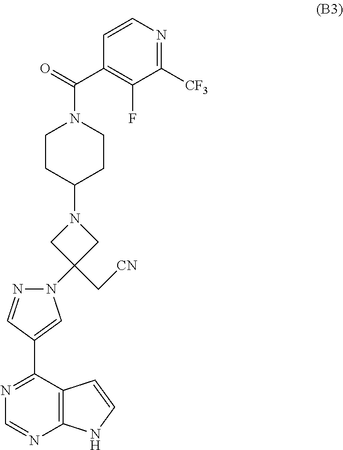 [SMILES: N#CCC1(n2cc(-c3ncnc4[nH]ccc34)cn2)CN(C2CCN(C(=O)c3ccnc(C(F)(F)F)c3F)CC2)C1]

Compound B4, which may be referred to as tofacitinib, (3R,4R)-1-4-methyl-3-(methyl-7H-pyrrolo[2,3-d]pyrimidin-4-ylamino)-β-oxo-piperidinepropanenitrile or 3-((3R,4R)-4-methyl-3-(methyl(7H-pyrrolo[2,3-d]pyrimidin-4-yl)amino)piperidin-1-yl)-3-oxopropanenitrile has the structure:

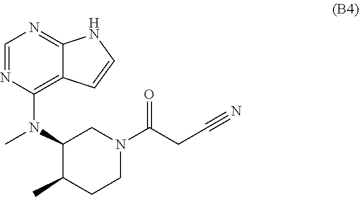 [SMILES: C[C@@H]1CCN(C(=O)CC#N)C[C@@H]1N(C)c1ncnc2[nH]ccc12]

Compound B5, which may be referred to as oclacitinib or N-methyl-1-((1r,4r)-4-(methyl(7H-pyrrolo[2,3-d]pyrimidin-4-yl)amino)cyclohexyl)methanesulfonamide, has the structure:

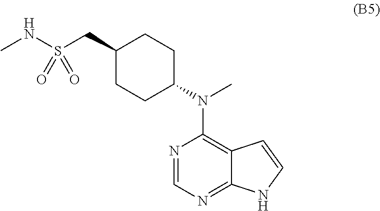 [SMILES: CNS(=O)(=O)C[C@H]1CC[C@H](N(C)c2ncnc3[nH]ccc23)CC1]

Compound B6, which may be referred to as ruxolotinib (INC424, INCB18424, JAKAFI®, JAKAVI®, available from Incyte Pharmaceuticals and Novartis) or (3R)-3-Cyclopentyl-3-[4-(7H-pyrrolo[2,3-d]pyrimidin-4-yl)-1H-pyrazol-1-yl]propanenitrile has the structure:

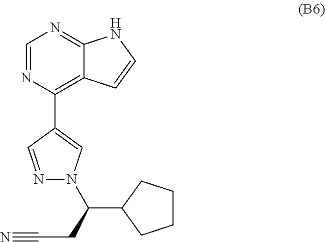 [SMILES: N#CC[C@H](C1CCCC1)n1cc(-c2ncnc3[nH]ccc23)cn1]

Compound B7, which may be referred to as baracitinib (LY3009104, INCB28050) 2-[1-ethylsulfonyl-3-[4-(7H-pyrrolo[2,3-d]pyrimidin-4-yl)pyrazol-1-yl]azetidin-3-yl]acetonitrile or 2-(3-(4-(7H-pyrrolo[2,3-d]pyrimidin-4-yl)-1H-pyrazol-1-yl)-1-(ethylsulfonyl)azetidin-3-yl)acetonitrile, has the structure:

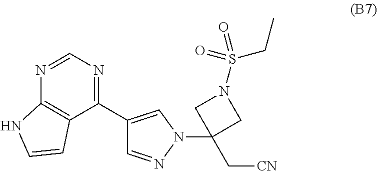 [SMILES: CCS(=O)(=O)N1CC(CC#N)(n2cc(-c3ncnc4[nH]ccc34)cn2)C1]

Compound B8, which may be referred to as lestaurtinib (CEP-701, KT5555, and A 154475.0)-2,3,9,10,11,12-hexahydro-10-hydroxy-10-(hydroxymethyl)-9-methyl-, (9S,10S,12R)-9,12-Epoxy-1H-diindolo[1,2,3-fg:3′, 2′,1′-kl]pyrrolo[3,4-i][1,6]benzodiazocin-1-one, has the structure:

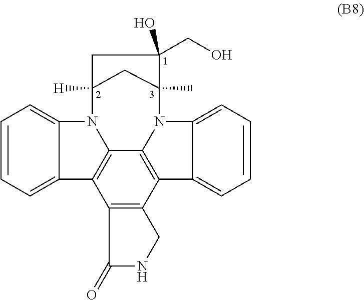 [SMILES: C[C@]12C[C@H](C[C@]1(O)CO)n1c3ccccc3c3c4c(c5c6ccccc6n2c5c31)CNC4=O]

Compound B9, which may be referred to as pacritinib (SB1518) or (16E)-11-[2-(1-Pyrrolidinyl)ethoxy]-14,19-dioxa-5,7,26-triazatetracyclo[19.3.1.12,6.18,12]heptacosa-1(25),2(26),3,5,8,10,12(27),16,21,23-decaene, has the structure:

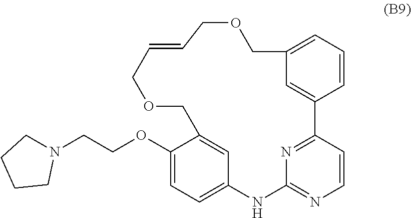 [SMILES: C1=C/COCc2cc(ccc2OCCN2CCCC2)Nc2nccc(n2)-c2cccc(c2)COC/1]

Compound B10, which may be referred to as TG101348, SAR302503, N-tert-Butyl-3-{5-methyl-2-[4-(2-pyrrolidin-1-yl-ethoxy)-phenylamino]-pyrimidin-4-ylamino}-benzenesulfonamide, or N-(tert-butyl)-3-((5-methyl-2-((4-(2-(pyrrolidin-1-yl)ethoxy)phenyl)amino)pyrimidin-4-yl)amino)benzenesulfonamide, has the structure:

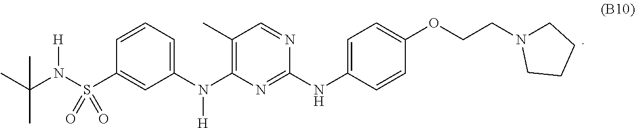 [SMILES: Cc1cnc(Nc2ccc(OCCN3CCCC3)cc2)nc1Nc1cccc(S(=O)(=O)NC(C)(C)C)c1]

Compound B11, which may be referred to as JSI-124, Cucurbitracin, Elatericin B, NSC-521777, (8S,9S,10R,13R,14R,16R,17R)-17-((R,E)-2,6-dihydroxy-6-methyl-3-oxohept-4-en-2-yl)-2,16-dihydroxy-4,4,8,9,13,14-hexamethyl-7,8,9,10,12,13,14,15,16,17-decahydro-3H-cyclopenta[a]phenanthrene-3,11(4H)-dione or 2,16α,20,25-tetrahydroxy-9-methyl-19-Nor-93,10a-lanosta-1,5,23-triene-3,11,22-trione, has the structure:

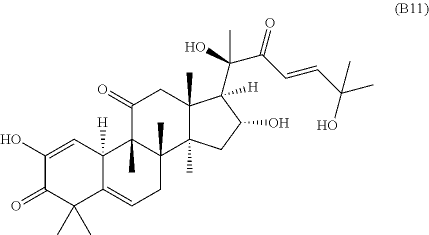

Additional JAK inhibitor compounds that may be used in the combinations, methods, kits, and articles of manufacture herein include GSK2586184, VX-509, INCB16562, XL019, NVP-BSK805, CEP33779, R-348, AC-430, CDP-R723 or BMS911543, NVP-BSK805, CEP33779, as well as those disclosed in U.S. Pat. No. 7,879,844, and the JAK inhibitor cyclodextrin-based polymer conjugates described in U.S. 2014-0357557.

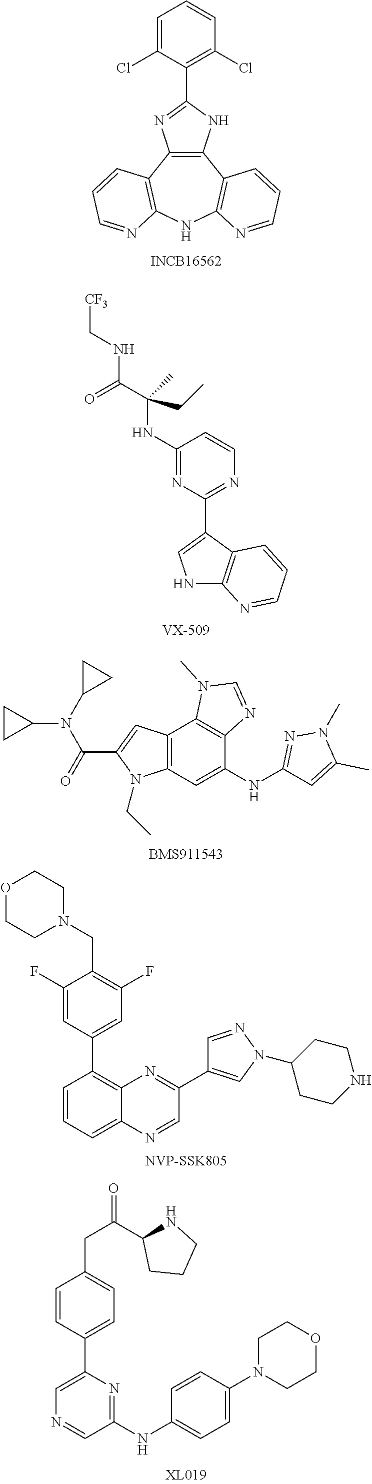

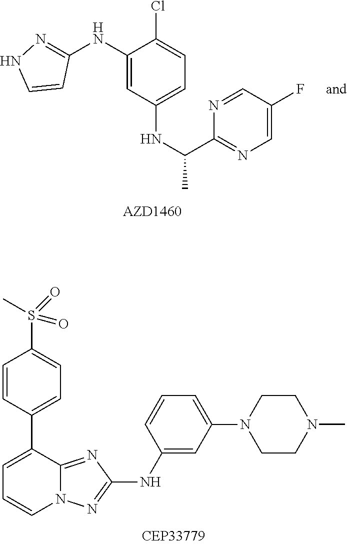 [SMILES: C[C@H](Nc1ccc(Cl)c(Nc2cc[nH]n2)c1)c1ncc(F)cn1 ; CN1CCN(c2cccc(Nc3nc4c(-c5ccc(S(C)(=O)=O)cc5)cccn4n3)c2)CC1]

In some embodiments, Compound B1, or a pharmaceutically acceptable salt thereof, is used in combination with Compound A1, or a pharmaceutically acceptable salt or hydrate thereof. In other embodiments, Compound B2, or a pharmaceutically acceptable salt thereof, is used in combination with Compound A1, or a pharmaceutically acceptable salt or hydrate thereof. In yet other embodiments, Compound B3, or a pharmaceutically acceptable salt thereof, is used in combination with Compound A1, or a pharmaceutically acceptable salt or hydrate thereof. In yet other embodiments, Compound B4, or a pharmaceutically acceptable salt thereof, is used in combination with Compound A1, or a pharmaceutically acceptable salt or hydrate thereof. In yet other embodiments, Compound B5, or a pharmaceutically acceptable salt thereof, is used in combination with Compound A1, or a pharmaceutically acceptable salt or hydrate thereof. In still another embodiment, Compound B6, or a pharmaceutically acceptable salt thereof, is used in combination with Compound A1, or a pharmaceutically acceptable salt or hydrate thereof. In another embodiment, Compound B7, or a pharmaceutically acceptable salt thereof, is used in combination with Compound A1, or a pharmaceutically acceptable salt or hydrate thereof. In yet other embodiments, Compound B8, or a pharmaceutically acceptable salt thereof, is used in combination with Compound A1, or a pharmaceutically acceptable salt or hydrate thereof. In further embodiments, Compound B9, or a pharmaceutically acceptable salt thereof, is used in combination with Compound A1, or a pharmaceutically acceptable salt or hydrate thereof. In yet other embodiments, Compound B10, or a pharmaceutically acceptable salt thereof, is used in combination with Compound A1, or a pharmaceutically acceptable salt or hydrate thereof. In other embodiments, Compound B11, or a pharmaceutically acceptable salt thereof, is used in combination with Compound A1, or a pharmaceutically acceptable salt or hydrate thereof.

Reference herein to “Compounds B1-B11”, “B1 to B11”, or “B1 through B11” is understood to include the full group of B1, B2 B3, B4, B5, B6, B7, B8, B9, B10, and B11. Compounds B1-B11 are commercially available or their methods of synthesis are generally known in the art. For instance, tofacitinib may be prepared as described in U.S. Pat. No. 6,956,041, filgotinib may be prepared by the methods seen in U.S. Pat. No. 8,853,240 and US 2015/0225398A1, INCB-039110 (INCB-39110) may be prepared by the methods seen in US 2011/112662 and US 2015/1246046, peficitinib may be prepared as described in U.S. Pat. Nos. 7,879,844 and 8,779,140, and momelotinib may be prepared as described in U.S. Pat. No. 8,486,941.

In one embodiment, the JAK inhibitor is selected from the group of momelotinib (CYT0387), ruxolitinib, fedratinib, baricitinib, lestaurtinib, pacritinib, XL019, AZD1480, LY2784544, BMS911543, and NS018, or a pharmaceutically acceptable salt thereof. In one embodiment, the JAK inhibitor is selected from the group of TG101348, JS-124, and INCB39110, CHZ868, and GSK2586184, or a pharmaceutically acceptable salt thereof. In another variation, the JAK inhibitor is momelotinib, or a pharmaceutically acceptable hydrochloride salt thereof. In another variation, the JAK inhibitor is filgotinib, or a pharmaceutically acceptable salt thereof.

### ASK1 Inhibitors

In some variations, the ASK1 inhibiting compound is a compound of Formula I:

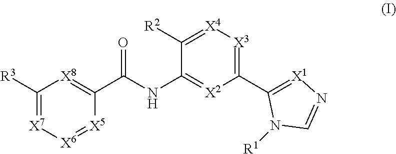

Wherein:

R¹ is selected from alkyl of 1-10 carbon atoms, alkenyl of 2-10 carbon atoms, alkynyl of 2-10 carbon atoms, cycloalkyl of 3-8 carbon atoms, aryl, heteroaryl, or heterocyclyl, all of which are optionally substituted with 1, 2, or 3 substituents selected from halo, oxo, alkyl, cycloalkyl, heterocyclyl, aryl, aryloxy, —NO₂, R⁶, —C(O)R⁶, —OC(O)—R⁶, —OC(O)—O—R⁶, —C(O)—N(R⁶)(R⁷), —S—R⁶, —S(═O)—R⁶, —S(═O)₂—R⁶, —S(═O)₂—N(R⁶R⁷), —S(═O)₂—O—R⁶, —N(R⁶R⁷), —N(R⁶)—C(O)—R⁷, —N(R⁶)—C(O)—O—R⁷, —N(R⁶)—C(═O)N(R⁶R⁷), —N(R)—S(═O)₂-R⁶, CN, and —OR⁶;

wherein alkyl, cycloalkyl, heterocyclyl, phenyl, and phenoxy are optionally substituted by 1, 2, or 3 substituents selected from alkyl, cycloalkyl, alkoxy, hydroxyl, and halo;

wherein R⁶ and R⁷ are independently selected from the group consisting of hydrogen, C₁-C₁₅ alkyl, cycloalkyl, heterocyclyl, aryl, and heteroaryl, all of which are optionally substituted with 1-3 substituents selected from halo, alkyl, mono- or dialkylamino, alkyl or aryl or heteroaryl amide, —CN, lower alkoxy, —CF₃, aryl, and heteroaryl; or

R⁶ and R⁷ when taken together with the nitrogen to which they are attached form a heterocycle;

R² is hydrogen, halo, cyano, alkoxy, or alkyl optionally substituted by halo;

R³ is aryl, heteroaryl, or heterocyclyl, all of which are optionally substituted with one or more substituents selected from alkyl of 1-6 carbon atoms, alkoxy of 1-6 carbon atoms, cycloalkyl of 3-8 carbon atoms, cycloalkylalkyl, aryl, arylalkyl, heteroaryl, heteroarylalkyl, heterocyclyl, heterocyclylalkyl, halo, oxo, —NO₂, haloalkyl, haloalkoxy, —CN, —O—R⁶, —OC(O)—R⁶, —OC(O)—O—R⁶, —C(O)—N(R⁶)(R⁷), —S—R⁶, —N(R⁶(R⁷), —S(═O)—R⁶, —S(═O)₂—R⁶, —S(═O)₂—N(R⁶)(R⁷), —S(═O)—O—R⁶, —N(R⁶)—C(O)—R⁷, —N(R⁶)—C(═O)N(R⁶)(R⁷), —N(R⁶)—C(O)—O—R⁷, —N(R⁶)—C(═O)N(R⁶)(R⁷), —C(O)R⁶, —C(O)—O—R⁶, —C(O)—N(R⁶R⁷), and —N(R⁶)—S(═O)2-R⁷, wherein the alkyl, alkoxy, cycloalkyl, aryl, heteroaryl, or heterocyclyl is further optionally substituted with one or more substituents selected from halo, oxo, —NO₂, alkyl, haloalkyl, haloalkoxy, —N(R⁶)(R⁷), —C(O)R⁶, —OC(O)—R⁶, —C(O)—N(R⁶)(R⁷), —CN, —O—R⁶, cycloalkyl, aryl, heteroaryl, and heterocyclyl;

with the proviso that the heteroaryl or heterocyclyl moiety includes at least one ring nitrogen atom; X¹, X², X³, X⁴, X⁵, X⁶, X⁷, and X⁸ are independently C(R⁴) or N in which each R⁴ is independently hydrogen, hydroxyl, halo, alkyl of 1-6 carbon atoms, alkoxy of 1-6 carbon atoms, or cycloalkyl of 3-8 carbon atoms, aryl, heteroaryl, heterocyclyl, halo, —NO₂, haloalkyl, haloalkoxy, —CN, —O—R⁶, —S-R⁶, —N(R⁶)(R⁷), —S(═O)—R⁶, —S(═O)₂—R⁶, —S(═O)₂—N(R⁶)(R⁷), —S(═O)—O—R⁶, —N(R⁶)—C(O)—R⁶, —N(R⁶)—C(O)—O—R⁷, —N(R⁶)—C(═O)N(R⁶R⁷), —C(O)—R⁶, —C(O)—O—R⁶, —C(O)—N(R⁶)(R⁷), —N(R⁶)—S(═O)₂—R₇, wherein the alkyl, cycloalkyl, aryl, heteroaryl, and heterocyclyl is further optionally substituted with one or more substituents selected from halo, oxo, —NO₂, —CF₃, —O-CF₃, —N(R⁶)(R⁷), —C(O)—R⁶, —C(O)—O—R⁷, —C(O)—N(R⁶)(R⁷), —CN, —CO—R⁶; or

X⁵ and X⁶ or X⁶ and X⁷ are joined to provide optionally substituted fused aryl or optionally substituted fused heteroaryl; and with the proviso that at least one of X², X³, and X⁴ is C(R⁴); at least two of X⁵, X⁶, X⁷, and X⁸ are is C(R⁴); and at least one of X², X³, X⁴, X⁵, X⁶, X⁷, and X⁸ is N; or a pharmaceutically acceptable salt or hydrate thereof.

An embodiment within each of the methods herein in which a compound of Formula I is used comprises use of a compound of Formula I, as described above, or a pharmaceutically acceptable salt or hydrate thereof, wherein R³ is selected from the group of:

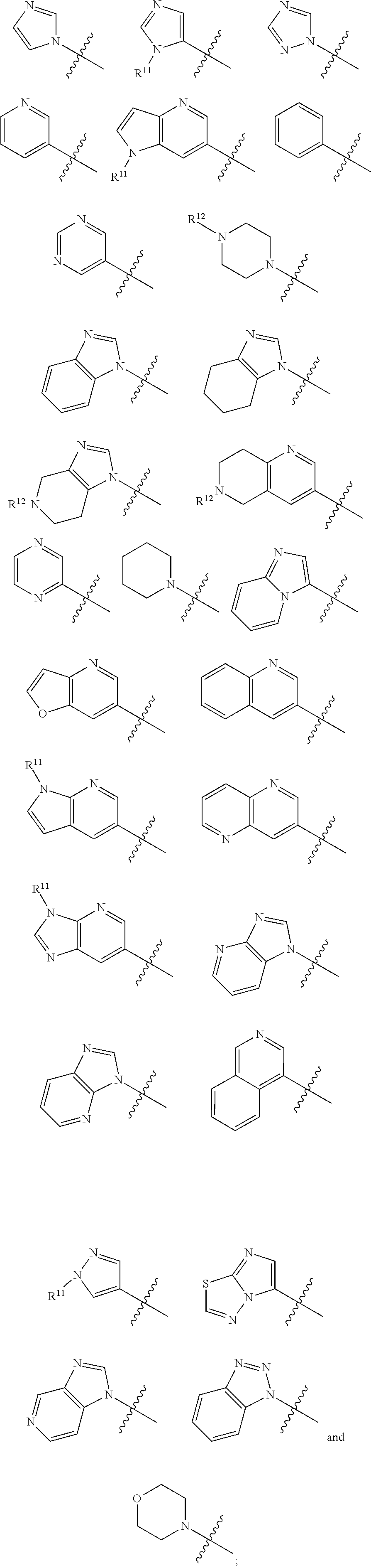

wherein:

R¹¹ is selected from hydrogen, alkyl of 1-6 carbon atoms, or cycloalkyl of 3-8 carbon atoms, wherein alkyl and cycloalkyl are optionally substituted by hydroxyl or halo;

R¹² is selected from hydrogen, alkyl of 1-6 carbon atoms, or cycloalkyl of 3-8 carbon atoms, —S(═O)—R⁶ or, —S(═O)₂—R⁶, wherein the alkyl and cycloalkyl are optionally substituted by hydroxyl or halo.

Another embodiment comprises use in the methods herein of a compound of Formula I, as described above, or a pharmaceutically acceptable salt or hydrate thereof, in which X¹, X², and X⁵ are all N, and X³, X⁴, X⁶, X⁷, and X⁸ are C(R⁴). This embodiment includes compounds in which R¹ is optionally substituted alkyl of from 1 to 6 carbon atoms, optionally substituted cycloalkyl of from 3 to 8 carbon atoms, or an optionally substituted heterocyclyl, particularly when the optional substituents are 1, 2, or 3 substituents chosen from hydroxyl, halo, or cycloalkyl of from 3 to 8 carbon atoms. Within the embodiment another embodiment includes compounds in which R³ is optionally substituted aryl, optionally substituted heteroaryl, or optionally substituted heterocyclyl, wherein the heteroaryl or heterocyclyl moieties contain, 1, 2, or 3 ring nitrogen atoms, and the aryl, heteroaryl, and heterocyclyl moieties are optionally substituted by alkyl of from 1 to 6 carbon atoms, cycloalkyl of from 3 to 8 carbon atoms, halo, cyano, or —OR⁶, in which alkyl and cycloalkyl are optionally substituted by hydroxyl or halo. A preferred group of R³ moieties includes those non-limiting examples described above.

Another embodiment includes use in the methods herein of a compound of Formula I in which X¹ and X⁵ are N, and X², X³, X⁴, X⁵, X⁶, X⁷, and X⁸ are C(R4). This group includes compounds in which R¹ is optionally substituted alkyl of from 1 to 6 carbon atoms, optionally substituted cycloalkyl of from 3 to 8 carbon atoms, or optionally substituted heterocyclyl, particularly where the optional substituents are 1, 2, or 3 substituents chosen from hydroxyl, halo, or cycloalkyl of from 3 to 8 carbon atoms. Within this group, a subgroup includes compounds in which R³ is optionally substituted aryl, optionally substituted heteroaryl, or optionally substituted heterocyclyl, wherein the heteroaryl or heterocyclyl moieties contain 1, 2, or 3 ring nitrogen atoms, and the aryl, heteroaryl, and heterocyclyl moieties contain 1, 2, or 3 ring nitrogen atoms, and the aryl, heteroaryl, and heterocyclyl moieties are optionally substituted by alkyl of from 1 to 6 carbon atoms, cycloalkyl of from 3 to 8 carbon atoms, halo, cyano, or —OR⁶, in which alkyl and cycloalkyl are optionally substituted by hydroxyl or halo.

Another embodiment provides use in the methods herein of a compound of Formula I in which X¹ and X² are N and X³, X⁴, X⁵, X⁶, X⁷, and X⁸ are C(R4). This group includes compounds in which R1 is optionally substituted alkyl of from 1 to 6 carbon atoms, optionally substituted cycloalkyl of from 3 to 8 carbon atoms, or optionally substituted heterocyclyl, particularly where the optional substituents are 1, 2, or 3 substituents chosen from hydroxyl, halo, or cycloalkyl. Within this group, a subgroup includes compounds in which R3 is optionally substituted aryl, optionally substituted heteroaryl, optionally substituted heterocyclyl, wherein the heteroaryl or heterocyclyl moieties contain 1, 2, or 3 ring nitrogen atoms, and the aryl, heteroaryl, and heterocyclyl moieties are optionally substituted by alkyl of from 1 to 6 carbon atoms, cycloalkyl of from 3 to 8 carbon atoms, halo, cyano, or —OR⁶, in which the alkyl and cycloalkyl are optionally substituted by hydroxyl or halo.

Another embodiment includes use in the methods herein of a compound of Formula I in which X¹ is C(R⁴). This group includes compounds in which R¹ is optionally substituted alkyl of from 1 to 6 carbon atoms, optionally substituted cycloalkyl of from 3 to 8 carbon atoms, or optionally substituted heterocyclyl, particularly where the optional substituents are chosen from hydroxyl, halo, or cycloalkyl of from 3 to 8 carbon atoms. Within this group, a subgroup includes compounds in which R³ is optionally substituted heteroaryl or optionally substituted heterocyclyl, wherein the heteroaryl or heterocyclyl moieties contain 1, 2, or 3 ring nitrogen atoms, and the aryl, heteroaryl, and heterocyclyl moieties are optionally substituted by alkyl of from 1 to 6 carbon atoms, cycloalkyl of from 3 to 8 carbon atoms, halo, cyano, or —OR⁶, in which the alkyl and cycloalkyl groups are optionally substituted by hydroxyl or halo.

The ASK1 inhibiting compounds for use in the methods herein include, but are not limited to, those compounds named below, which may be prepared by the methods described in U.S. Pat. Nos. 8,552,196 and 8,742,126, which are incorporated herein by reference:
5-(2,5-difluorophenyl)-N-(3-(4-methyl-4H-1,2,4-triazol-3-yl)phenyl)nicotinamide;
4-(imidazo[1,2-a]pyridin-3-yl)-N-(3-(4-methyl-4H-1,2,4-triazol-3-yl)phenyl)-picolinamide;
4-(2-aminopyrimidin-5-yl)-N-(3-(4-cyclopropyl-4H-1,2,4-thiazol-3-yl)phenyl)picolinamide;
N-(3-(4-methyl-4H-1,2,4-triazol-3-yl)phenyl)-5-phenylnicotinamide;
N-(3-(4-cyclopropyl-4H-1,2,4-triazol-3-yl)phenyl)-4-phenylpicolinamide;
N-(3-(4-(tetrahydro-2H-pyran-4-yl)-4H-1,2,4-triazol-3-yl)phenyl)-3,4′-bipyridine 2′-carboxamide;
2-hydroxy-N-(3-(4-methyl-4H-1,2,4-triazol-3-yl)phenyl)-6-phenylpyrimidine-4-carboxamide;
N-(3-(4-cyclopropyl-4H-1,2,4-triazol-3-yl)phenyl)-3,4′-bipyridine-carboxamide;
N-(3-(4-cyclopropyl-4H-1,2,4-triazol-3-yl)phenyl)-4-(1H-imidazol-1-yl)picolinamide;
N-(3-(4-methyl-4H-1,2,4-triazol-3-yl)phenyl)-4-phenylpicolinamide;
N-(3-(4 (3-amino-3-oxopropyl)-4H-1,2,4-triazol-3-yl)phenyl)-3,4′-bipyridine-carboxamide;
N-(3-(4-cyclopropyl-4H-1,2,4-triazol-3-yl)phenyl)-4-(1H-1,2,4-triazol-3-yl)picolinamide;
N-(3-(4-methyl-4H-1,2,4-triazol-3-yl)phenyl)-6-phenylpicolinamide;
N-(3-(4-(2-acetamidoethyl)-4H-1,2,4-triazol-3-yl)phenyl)-3,4′-bipyridine-carboxamide;
N-(3-(4-methyl-4H-1,2,4-triazol-3-yl)phenyl)-4-(4-methylpiperazin-1-yl)picolinamide;
N-(3-(4-methyl-4H-1,2,4-triazol-3-yl)phenyl)-2,31-bipyridine-6-carboxamide;
N-(3-(4-methyl-4H-1,2,4-triazol-3-yl)phenyl)-4-morpholinopicolinamide;
N-(3-(4-methyl-4H-1,2,4-triazol-3-yl)phenyl)-6-(quinolin-6-yl)picolinamide;
(R)—N-(3-(4-(1-hydroxypropan-2-yl)-4H-1,2,4-triazol-3-yl)phenyl)-3,4′-bipyridine-2′-carboxamide;
N-(3-(4-cyclopropyl-4H-1,2,4-triazol-3-yl)phenyl)-6-hydroxy-3,4′-bipyridine-2′-carboxamide;
N-(3-(4-methyl-4H-1,2,4-triazol-3-yl)phenyl)-3,3′-bipyridine-5-carboxamide;
(S)—N-(3-(4-(1-hydroxypropan-2-yl)-4H-1,2,4-triazol-3-yl)phenyl)-3,4′-bipyridine-2′-carboxamide;
N-(3-(4-methyl-4H-1,2,4-triazol-3-yl)phenyl)-4-(3-oxopiperazin-1-yl)picolinamide;
N-(3-(4-methyl-4H-1,2,4-triazol-3-yl)phenyl)-3,4′-bipyridine-2′-carboxamide;
N-(3-(4-cyclopropyl-4H-1,2,4-triazol-3-yl)phenyl)-6-methoxy-3,4′-bipyridine-2′-carboxamide;
4-(3-aminopyrrolidin-1-yl)-N-(3-(4-methyl-4H-1,2,4-triazol-3-yl)phenyl)picolinamide;
N-(3-(4-methyl-4H-1,2,4-triazol-3-yl)phenyl)-2-phenylisonicotinamide;
6-amino-N-(3-(4-cyclopropyl-4H-1,2,4-triazol-3-yl)phenyl)-3,4′-bipyridine-2′-carboxamide;
(R)—N-(3-(4-(2-hydroxypropyl)-4H-1,2,4-triazol-3-yl)phenyl)-3,4′-bipyridine-2′-carboxamide;
5-methoxy-N-(3-(4-methyl-4H-1,2,4-triazol-3-yl)phenyl)-3,4′-bipyridine-2′-carboxamide;
methyl 2′-(3-(4-cyclopropyl-4H-1,2,4-triazol-3-yl)phenylcarbamoyl)-3,4′-bipyridin-6-ylcarbamate;
5-methoxy-N-(3-(4-methyl-4H-1,2,4-triazol-3-yl)phenyl)-3,4′-bipyridine-2′-carboxamide;
methyl 2′-(3-(4-cyclopropyl-4H-1,2,4-triazol-3-yl)phenylcarbamoyl)-3,4′-bipyridin-6-yl carbamate;
(S)—N-(3-(4-(2-hydroxypropyl)-4H-1,2,4-triazol-3-yl)phenyl)-3,4′-bipyridine-2′-carboxamide;
4-(1-methyl-1H-imidazol-5-yl)-N-(3-(4-methyl-4H-1,2,4-triazol-3-yl)phenyl)picolinamide;
N-(3-(4-cyclopropyl-4H-1,2,4-triazol-3-yl)phenyl)-4-(1-methyl-1H-imidazol-5-yl)picolinamide;
4-(1H-benzo[d]imidazol-1-A-N-(3-(4-cyclopropyl-4H-1,2,4-triazol-3-yl)phenyl)picolinamide;
N-(3-(4-cyclopropyl-4H-1,2,4-triazol-3-yl)phenyl)-4-(2,4-dimethoxypyrimidiN-5-yl)picolinamide;
N-(3-(4-((1-hydroxycyclopropyl)methyl)-4H-1,2,4-triazol-3-yl)phenyl)-3,4′-bipyridine-2′-carboxamide;
N-(3-(4-cyclopropyl-4H-1,2,4-triazol-3-yl)phenyl)-4-(4-phenyl-1H-imidazol-1-yl)picolinamide;
6-cyclopropyl-N-(3-(4-cyclopropyl-4H-1,2,4-triazol-3-yl)phenyl)-3,4′-bipyridine-2′-carboxamide;
(S)—N-(3-(4-(2-hydroxypropyl)-4H-1,2,4-triazol-3-yl)phenyl)-3,4′-bipyridine-2′-carboxamide;
N-(3-(4-cyclobutyl-4H-1,2,4-triazol-3-yl)phenyl)-3,4′-bipyridine-2′-carboxamide;
N2′-(3-(4-cyclopropyl-4H-1,2,4-triazol-3-yl)phenyl)-3,4′-bipyridine-2′,6-dicarboxamide;
(S)—N-(3-(4-(1,1,1-trifluoropropan-2-yl)-4H-1,2,4-triazol-3-yl)phenyl)-3,4′-bipyridine-2′-carboxamide;
N-(3-(4-cyclopentyl-4H-1,2,4-triazol-3-yl)phenyl)-3,4′-bipyridine-2′-carboxamide;
N-(3-(4-cyclopropyl-4H-1,2,4-triazol-3-yl)phenyl)-6-(trifluoromethyl)-3,4′-bipyridine-2′-carboxamide;
N2′-(3-(4-cyclopropyl-4H-1,2,4-triazol-3-yl)phenyl)-3,4′-bipyridine-2′,5-dicarboxamide;
N-(3-(4-cyclopropyl-4H-1,2,4-triazol-3-yl)phenyl)-4-(2-methyl-1H-imidazol-1-yl)picolinamide;
N-(3-(4-cyclopropyl-4H-1,2,4-triazol-3-yl)phenyl)-6-methyl-3,4′-bipyridine-2′-Carboxamide;
5-cyano-N-(3-(4-cyclopropyl-4H-1,2,4-triazol-3-yl)phenyl)-3,4′-bipyridine-2′-carboxamide;
N-(3-(4-cyclopropyl-4H-1,2,4-triazol-3-yl)phenyl)-4-(4-methyl-1H-imidazol-1-yl)picolinamide;
N-(6-(4-cyclopropyl-4H-1,2,4-triazol-3-yl)pyridin-2-yl)-3,4′-bipyridine-2′-carboxamide;
2-amino-N-(3-(4-cyclopropyl-4H-1,2,4-triazol-3-yl)phenyl)-3,4′-bipyridine-2′-carboxamide;
N-(3-(4-cyclopropyl-4H-1,2,4-triazol-3-yl)phenyl)-4-(4,5-dimethyl-1H-imidazol-1-yl)picolinamide;
N-(3-(4-((1 S,2S)-2-methylcyclopropyl)-4H-1,2,4-triazol-3-yl)phenyl)-3,4)-bipyridine-2′-carboxamide;
N-(3-(4-cyclopropyl-4H-1,2,4-triazol-3-yl)phenyl)-2-methoxy-34′-bipyridine-2′-carboxamide;
N-(3-(4-cyclopropyl-4H-1,2,4-triazol-3-yl)phenyl)-4-(4-(trifluoromethyl)-1H-imidazol-1-yl)picolinamide;
N-(3-(4-cyclopropyl-4H-1,2,4-triazol-3-yl)phenyl)-6-(2,2,2-trifluoroethoxy)-3,4′-bipyridine-2′-carboxamide;
N-(3-(4-cyclopropyl-4H-1,2,4-triazol-3-yl)phenyl)-4-(1-methyl-1H-pyrazol-4-yl)picolinamide;
N-(3-(4-cyclopropyl-4H-1,2,4-triazol-3-yl)phenyl)-4-(2-methoxypyrimidin-5-yl)picolinamide;
N-(3-(4-cyclopropyl-4H-1,2,4-triazol-3-yl)phenyl)-4-methyl-3,4′-bipyridine-2′-Carboxamide;
N-(3-(4-cyclopropyl-4H-1,2,4-triazol-3-yl)phenyl)-4-(imidazo[1,2-a]pyridin-3-yl)picolinamide;
ethyl-N-(3-(4-methyl-4H-1,2,4-triazol-3-yl)phenyl)-3,4′-bipyridine-2′-carboxamide;
N-(3-(4-(2,2,2-trifluoroethyl)-4H-1,2,4-triazol-3-yl)phenyl)-3,4′-bipyridine-2′-carboxamide;
6-chloro-[32′,5′,4″]terpyridine-2″-carboxylic acid[3-(4-cyclopropyl-4H-[1,2,4]triazol-3-yl)-phenyl]amide
N-(3-(4-cyclopropyl-4H-1,2,4-triazol-3-yl)phenyl)-6-(pyrrolidin-1-yl)-3,4′-bipyridine-2′-Carboxamide;
N-(3-(4-cyclopropyl-4H-1,2,4-triazol-3-yl)phenyl)-5-(trifluoromethyl)-3,4′-bipyridine-2′-carboxamide;
N-(3-(1-cyclopropyl-1H-imidazol-5-yl)phenyl)-3,4′-bipyridine-2′-carboxamide;
N-(3-(4-cyclopropyl-4H-1,2,4-triazol-3-yl)phenyl)-4-(1,2-dimethyl-1H-imidazol-5-yl)picolinamide;
4-(1H-benzo[d] [1,2,3]triazol-yl)-N-(3-(4-cyclopropyl-4H-1,2,4-triazol-3-yl)phenyl) picolinamide;
N-(3-(4-cyclopropyl-4H-1,2,4-triazol-3-yl)phenyl)-4-(4-sulfamoylphenyl)picolinamide;
5 N-(3-(4-cyclopropyl-4H-1,2,4-triazol-3-yl)phenyl)-5-methoxy-3,4′-bipyridine-2′-carboxamide;
N-(3-(4-cyclopropyl-4H-1,2,4-triazol-3)phenyl)-6-fluoro-5-methyl-3,4′-bipyridine-2′-carboxamide;
N-(3-(4-cyclopropyl-4H-1,2,4-triazol-3-yl)phenyl)-5-fluoro-3,4′-bipyridine-2′-carboxamide;
N-(3-(4-cyclopropyl-4H-1,2,4-triazol-3-yl)phenyl)-2-methyl-3,4′-bipyridine-2′-Carboxamide;
N-(3-(4-cyclopropyl-4H-1,2,4-triazol-3-yl)phenyl)-4-(4,5,6,7-tetrahydro-1H-benzo[d]imidazol-1-yl)picolinamide;
N-(3-(4-cyclopropyl-4H-1,2,4-triazol-3-yl)phenyl)-4-(4-(Nmethylsulfamoyl)phenyl) picolinamide;
N5-ter-butyl-N2′-(3(4-cyclopropyl-4H-1,2,4-triazol-3-yl)phenyl)-3,4′-bipyridine-2′,5-dicarboxamide;
N-(3-(4-cyclopropyl-4H-1,2,4-triazol-3-yl)phenyl)-4-(pyrazin-2-yl)picolinamide;
N-(3-(4-cyclopropyl-4H-1,2,4-triazol-3-yl)phenyl)-4-(4-(Nisopropylsulfamoyl) phenyl)picolinamide;
chloro-N-(3-(4-cyclopropyl-4H-1,2,4-triazol-3-yl)phenyl)-3,4′-bipyridine-2′-carboxamide;
4-(1H-benzo[d]imidazol-1-yl)-N-(3-(1-cyclopropyl-1H-imidazol-5-yl)phenyl)picolinamide;
6-cyclopropyl-N-(6-(4-cyclopropyl-4H-1,2,4-triazol-3-yl)pyridin-2-yl)-3,4′-bipyridine-2′-carboxamide;
N-(3-(4-cyclopropyl-4H-1,2,4-triazol-3-yl)phenyl)-4-(3-(methylsulfonyl)phenyl)picolinamide;
N-(3-(4-cyclopropyl-4H-1,2,4-triazol-3-yl)phenyl)-4-(isoquinolin-4-yl)picolinamide;
N-(6-(4-cyclopropyl-4H-1,2,4-triazol-3-yl)pyridin-2-yl)-4-(4-(methylsulfonyl)phenyl)picolinamide;
N-(3-(4-cyclopropyl-4H-1,2,4-triazol-3-yl)phenyl)-4-(2-(methylsulfonyl)phenyl)picolinamide;
N-(3-(4-cyclopropyl-4H-1,2,4-triazol-3-yl)phenyl)-4-(1,5-dimethyl-1H-pyrazol-4-yl)picolinamide;
6-cyclobutyl-N-(3-(4-cyclopropyl-4H-1,2,4-triazol-3-yl)phenyl)-3,4′-bipyridine-2′-carboxamide;
N-(3-(4-cyclopropyl-4H-1,2,4-triazol-3-yl)phenyl)-6-isopropyl-3,4′-bipyridine-2′-carboxamide;
N-(3-(4-cyclopropyl-5H-1,2,4-triazol-3-yl)phenyl)-4-(4-(methylsulfonyl)phenyl)picolinamide;
N-(3-(4-cyclopropyl-4H-1,2,4-triazol-3-yl)phenyl)-6-(dimethylamino)-3,4′-bipyridine-2′-carboxamide;
N-(3-(4-cyclopropyl-4H-1,2,4-triazol-3-yl)phenyl)-4-(pyridin-3-yl)quinoline-2-carboxamide;
N-(3-(4-cyclopropyl-4H-1,2,4-triazol-3-yl)phenyl)-4-(1Hpyrrolo[2,3-b]pyridin-5-yl)picolinamide;
6-cyclopropoxy-N-(3-(4-cyclopropyl-4H-1,2,4-triazol-3-yl)phenyl)-3,4′-bipyridine-2′-carboxamide;
N-(3-(4-cyclopropyl-4H-1,2,4-triazol-3-yl)phenyl)-4-(1H-imidazo[4,5-b]pyridin-1-yl)picolinamide;
N-(3-(4-cyclopropyl-4H-1,2,4-triazol-3-yl)phenyl)-6-fluoro-3,4′-bipyridine-2′-carboxamide;
N-(3-(4-cyclopropyl-4H-1,2,4-triazol-3-yl)phenyl)-4-(4-(2-oxoimidazolidin-1-yl)phenyl)picolinamide;
N-(3-(4-cyclopropyl-4H-1,2,4-triazol-3-yl)phenyl)-4-(3Himidazo[4, 5-1)]pyridin-3-yl)picolinamide;
N-(3-(4-cyclopropyl-4H-1,2,4-triazol-3-yl)phenyl)-6-isopropoxy-3,4′-bipyridine-2′-carboxamide;
N-(3-(4-cyclopropyl-4H-1,2,4-triazol-3-yl)phenyl)-6-ethyl-3,4′-bipyridine-2′-carboxamide;
N-(3-(4-cyclopropyl-4H-1,2,4-triazol-3-yl)phenyl)-4-(1H-imidazo[4,5-c]pyridin-1-yl)picolinamide;
6-cyclobutoxy-N-(3-(4-cyclopropyl-4H-1,2,4-triazol-3-yl)phenyl)-3,4′-bipyridine-2′-carboxamide;
6-cyclopropyl-N-(3-(1-cyclopropyl-1H-imidazol-5-yl)phenyl)-3,4′-bipyridine-2′-carboxamide;
N-(3-(4-cyclopropyl-4H-1,2,4-triazol-3-yl)phenyl)-4-(quinolin-3-yl)picolinamide;
N-(3-(4-cyclopropyl-4H-1,2,4-triazol-3-yl)phenyl)-4-(4-(Ncyclopropylsulfamoyl) phenyl)picolinamide;
N-(3-(1-cyclopropyl-1H-imidazol-5-yl)phenyl)-4-(quinolin-3-yl)picolinamide;
6-cyclopentyl-N-(3-(4-cyclopropyl-4H-1,2,4-triazol-3-yl)phenyl)-3,4′-bipyridine-2′-carboxamide;
N-(3-(4-cyclopropyl-4H-1,2,4-triazol-3-yl)phenyl)-4-(imidazo[2, 1-b] [1,3,4]thiadiazol-5-yl)picolinamide;
N-(3-(4-cyclopropyl-4H-1,2,4-triazol-3-yl)phenyl)-4-(5-cyclopropylpyrazin-2-yl)picolinamide;
N-(3-(4-cyclopropyl-4H-1,2,4-triazol-3-yl)phenyl)-6-(1-methyl-2-oxopyrrolidin-3-yl)-3,4′-bipyridine-2′-Carboxamide;
4-(4-chloro-1H-imidazol-1-yl)-N-(3-(4-cyclopropyl-4H-1,2,4-triazol-3-yl)phenyl)picolinamide;
6-cyclopropyl-N-(3-(4-cyclopropyl-4H-1,2,4-triazol-3-yl)phenyl)-5-fluoro-3,4′-bipyridine-2′-carboxamide;
(S)-4-(4-cyclopropyl-1H-imidazol-1-yl)-N-(3-(4-(3-methylbutan-2-yl)-4H-1,2,4-triazol-3-yl)phenyl)picolinamide;
6-cyclopropyl-N-(3-(4-cyclopropyl-4H-1,2,4-triazol-3-yl)phenyl)-2,31-bipyridine-6-carboxamide;
6-cyclopropyl-N-(3-(4-cyclopropyl-4H-1,2,4-triazol-3-yl)phenyl)-2,31-bipyridine-4-carboxamide;
N-(6-(4-cyclopropyl-4H-1,2,4-triazol-3-yl)pyridin-2-yl)-5-(6-cyclopropylpyridin-3-yl)-2,4-difluorobenzamide;
6-cyclopropyl-N-(6-(4-cyclopropyl-4H-1,2,4-triazol-3-yl)pyridin-2-yl)-2,31-bipyridine-4-carboxamide;
6-cyclopropyl-N-(6-(4-cyclopropyl-4H-1,2,4-triazol-3-yl)pyridin-2-yl)-3,31-bipyridine-5-carboxamide;
6-cyclopropyl-N-(6-(4-cyclopropyl-4H-1,2,4-triazol-3-yl)pyridin-2-yl)-2,31-bipyridine-6-carboxamide;
N-(3-(4-cyclopropyl-41-1,2,4-triazol-3-yl)phenyl)-4-(5-methyl-4-(trifluoromethyl)-4,5,6, 7-tetrahydro-1H-imidazo[4,5-c]pyridin-1-yl)picolinamide;
N-(6-(4-cyclopropyl-4H-1,2,4-triazol-3-yl)pyridin-2-yl)-4-(5-methyl-4-(trifluoromethyl)-4,5,6, 7-tetrahydro-1H-imidazo[4,5-c-]pyridin-1-yl)picolinamide;
4-(5-cyclopropyl-4-methyl-4H-1,2,4-triazol-3-yl)-N-(3-(4-cyclopropyl-4H-1,2,4-triazol-3-yl)phenyl)picolinamide;
4-(3-cyclopropyl-1,2,4-oxadiazol-5-yl)-N-(3-(4-cyclopropyl-4H-1,2,4-triazol-3-yl)phenyl)picolinamide;
N-(3-(4-cyclopropyl-4H-1,2,4-triazol-3-yl)phenyl)-4-(3-methyl-1,2,4-oxadiazol-5-yl)picolinamide;
6-cyclopropyl-N-(3-(4-(3-hydroxybutan-2-yl)-4H-1,2,4-triazol-3-yl)phenyl)-3,4′-bipyridine-2′-carboxamide;
4-chloro-N-(6-(4-cyclopropyl-4H-1,2,4-triazol-3-yl)pyridin-2-yl)-5-(6-cyclopropylpyridin-3-yl)-2-fluoro benzamide;
6-cyclopropyl 1-N-(6-(4-((2S,3 R)-3-hydroxybutan-2-yl)-4H-1,2,4-triazol-3-yl)pyridin-2-yl)-3,4′-bipyridine-2′-carboxamide;
6-cyclopropyl 1-N-(6-(4-((2S,3 S)-3-hydroxybutan-2-yl)-4H-1,2,4-triazol-3-yl)pyridin-2-yl)-3,4′-bipyridine-2′-carboxamide;
6-cyclopropyl-N-(6-(4-(1-(pyrolidin-1-yl)propan-2-yl)-4H-1,2,4-triazol-3-yl)pyridin-2-yl)-3,4′-bipyridine-2′-carboxamide;
N-(3-(4-cyclopropyl-4H-1,2,4-triazol-3-yl)phenyl)-4-(1-(2,2,2-trifluoroethyl)-1H-pyrrolo[3,2-b]pyridin-6-yl)picolinamide;
N-(3-(4-cyclopropyl-4H-1,2,4-triazol-3-yl)phenyl)-4-(isopropyl-1-pyrrolo[3,2-b]pyridin-6-yl)picolinamide;
S)-6-cyclopropyl-N-(3-(4-(3,3-dimethylbutan-2-yl)-4H-1,2,4-triazol-3-yl)phenyl)-3,4′-bipyridine-2′-carboxamide;
6-cyclopropyl-N-(6-(4-(1-methylpiperidin-4-yl)-4H-1,2,4-triazol-3-yl)pyridin-2-yl)-3,4′-bipyridine-2′-carboxamide;
N-(3-(4-sec-butyl-4H-1,2,4-triazol-3-yl)phenyl)-6-cyclopropyl-3,4′-bipyridine-2′-carboxamide;
(S)-6-cyclopropyl-N-(3-(4-(1-cyclopropylethyl)-4H-1,2,4-triazol-3-yl)phenyl)-3,4′-bipyridine-2′-carboxamide;
6-cyclopropyl-N-(3-(4-(pentan-3-yl)-4H-1,2,4-triazol-3-yl)phenyl)-3,4′-bipyridine-2′-carboxamide;
(S)-6-cyclopropyl-N-(3-(4-(1-methoxypropan-2-yl)-4H-1,2,4-triazol-3-yl)phenyl)-3,4′-bipyridine-2′-carboxamide;
6-cyclopropyl-N-(6-(4-cyclopropyl-4H-1,2,4-triazol-3-yl)pyridin-2-yl)-6′-methyl-3,4′-bipyridine-2′-carboxamide;
(S)-6-cyclopropyl-N-(6-(4-(1-methoxypropan-2-yl)-4H-1,2,4-triazol-3-yl)pyridin-2-yl)-dine-2′-carboxamide;
(S)—N-(3-(4-sec-butyl-4H-1,2,4-triazol-3-yl)phenyl)-6-cyclopropyl-3,4′-bipyridine-2′-Carboxamide;
N-(6-(4-cyclopropyl-4H-1,2,4-triazol-3-yl)pyridin-2-yl)-3-(4-(2,2,2-trifluoro-1-methoxyethyl)-1H-imidazol-1-yl)benzamide;
N-(6-(4-cyclopropyl-4H-1,2,4-triazol-3-yl)pyridin-2-yl)-4-(6-cyclopropylpyridin-3-yl)-7,8-dimethyl quinoline-2-carboxamide;
(S)-6-cyclopropyl-N-(3-(4-(3-methylbutan-2-yl)-4H-1,2,4-triazol-3-yl)phenyl)-3,4′-bipyridine-2′-carboxamide;
(R)-6-cyclopropyl-N-(3-(4-(1-(2,6-dimethylphenoxy)propan-2-yl)-4H-1,2,4-triazol-3-yl)phenyl)-3,4′-bipyridine-2′-carboxamide;
N-(3-(4-cyclopropyl-4H-1,2,4-triazol-3-yl)phenyl)-4-(6-cyclopropylpyridin-3-yl)-7,8-dimethylquinoline-2-carboxamide;
3-(4-cyclopropyl-1H-imidazol-1-yl)-N-(6-(4-cyclopropyl-4H-1,2,4-triazol-3-yl)pyridin-2-yl)-4-methoxybenzamide;
4-chloro-3-(4-cyclopropyl-1H-imidazol-1-yl)-N-(6-(4.-cyclopropyl-4H-1,2,4-triazol-3-yl)pyridin-2-yl)benzamide;
4-(4-cyclopropyl-1H-imidazol-1-yl)-N-(6-(4-cyclopropyl-4H-1,2,4-triazol-3-yl)pyridin-2-yl)quinoline-2-carboxamide;
N-(6-(4-cyclopropyl-4H-1,2,4-triazol-3-yl)pyridin-2-yl)-4-(6-cyclopropylpyridin-3-yl)quinoline-2-carboxamide;
N-(6-(4-cyclopropyl-4H-1,2,4-triazol-3-yl)pyridin-2-yl)-5-(6-cyclopropylpyridin-3-yl)-2-fluorobenzamide;
(S)-6-cyclopropyl-N-(3-(4-(1, 1, 1-trifluoropropan-2-yl)-4H-1,2,4-triazol-3-yl)phenyl)-3,4′-bipyridine-2′-carboxamide;
(S)-tert-butyl 2-(3-(3-(6-cyclopropyl-3,4′-bipyridine-2′-carboxamido)phenyl)-4H-1,2,4-triazol-4-yl)propanoate;
N-(3-(4-cyclobutyl-4H-1,2,4-triazol-3-yl)phenyl)-6-cyclopropyl-3,4′-bipyridine-2′-carboxamide;
(S)-6-cyclopropyl-N-(3-(4-(1-phenylethyl)-4H-1,2,4-triazol-3-yl)phenyl)-3,4′-bipyridine-2′-carboxamide;
6-cyclopropyl-N-(3-(4-isopropyl-4H-1,2,4-triazol-3-yl)phenyl)-3,4′-bipyridine-2′-carboxamide;
3-(4-cyclopropyl-1H-imidazol-1-yl)-N-(6-(4-isopropyl-4H-1,2,4-triazol-3-yl)pyridin-2-yl)benzamide;
N-(6-(4-cyclopropyl-4H-1,2,4-triazol-3-yl)pyridin-2-yl)-4-(4-(2,2,2-trifluoro-1-hydroxyethyl)-1H-imidazol-1-yl)picolinamide;
(S)-3-(4-cyclopropyl-1H-imidazol-1-yl)-N-(6-(4-(1-phenyl-ethyl)-4H-1,2,4-triazol-3-yl)pyridin-2-yl)benzamide;
N-(3-(4-cyclopropyl-4H-1,2,4-triazol-3-yl)phenyl)-4-(4-(2,2,2-trifluoro-1-hydroxyethyl)-1H-imidazol-1-yl)picolinamide;
N-(6-(1-cyclopropyl-1H-imidazol-5-yl)pyridin-2-yl)-4-(4,5-dimethyl-1H-imidazol-1-yl)picolinamide;
N-(6-(4-cyclopropyl-4H-1,2,4-triazol-3-yl)pyridin-2-yl)-3-(4-(2,2,2-trifluoro-1-hydroxyethyl)-1H-imidazol-1-yl)benzamide;
N-(6-(1-cyclopropyl-1H-imidazol-5-yl)pyridin-2-yl)-6-(2-benzamide;
hydroxypropan-2-yl)-3,4′-bipyridine-2′-carboxamide;
3-(4-cyclopropyl-1H-imidazol-1-yl)-N-(6-(4-cyclopropyl-4H-1,2,4-triazol-3-yl)pyridin-2-yl)-5-methylbenzamide;
N-(6-(4-cyclopropyl-4H-1,2,4-triazol-3-yl)pyridin-2-yl)-3-(4,5-dimethyl-1H-imidazol-1-yl)benzamide;
N-(3-(4-(cyclopropylmethyl)-4H-1,2,4-triazol-3-yl)phenyl)-3,4′-bipyridin carboxamide;
4-(4-cyclopropyl-2-methyl-1H-imidazol-1-yl)-N-(3-(4-cyclopropyl-4H-1,2,4-triazol-3-yl)phenyl)picolinamide;
4-(4-cyclopropyl-2-methyl-1H-imidazol-1-yl)-N-(6-(4-cyclopropyl-4H-1,2,4-triazol-3-yl)pyridin-2-yl)picolinamide;
4-(4-cyclopropyl-1H-imidazol-1-yl)-N-(3-(4-isopropyl-4H-1,2,4-triazol-3-yl)phenyl)picolinamide;
4-(4-cyclopropyl-1H-imidazol-1-yl)-N-(3-(4-(cyclopropylmethyl)-4H-1,2,4-triazol-3-yl)phenyl)picolinamide;
4-(4-cyclopropyl-1-imidazol-1-yl)-N-(3-(4-(1-phenylethyl)-4H-1,2,4-triazol-3-yl)phenyl)picolinamide;
N-(6-(4-cyclopropyl-4H-1,2,4-triazol-3-yl)pyridin-2-yl)-4-(4,5,6,7-tetrahydro-1H-benzo[d]imidazol-1-yl)picolinamide;
N-(6-(4-cyclopropyl-4H-1,2,4-triazol-3-yl)pyridin-2-yl)-4-(4-(trifluoromethyl)-1H-imidazol-1-yl)picolinamide;
N-(6-(4-cyclopropyl-4H-1,2,4-triazol-3-yl)pyridin-2-yl)-3-(4,5,6,7-tetrahydro-1-benzo[d]imidazol-1-yl)benzamide;
1-(3-(6-(4-cyclopropyl-4H-1,2,4-triazol-3-yl)pyridin-2-ylcarbamoyl)phenyl)-5-methyl-1H-imidazole-4-carboxylicacid;
(S)-3-(4-cyclopropyl-1H-imidazol-1-yl)-N-(6-(4-(1-phenylethyl)-4H-1,2,4-triazol-3-yl)pyridin-2-yl)benzamide;
6-cyclopropyl-N-(6-(4-cyclopropyl-4H-1,2,4-triazol-3-yl)pyridin-2-yl)-5′-methyl-3,4′-bipyridine-2′-carboxamide;
(S)-3-(4,5-d methyl-1H-imidazol-1-yl)-N-(6-(4-(1,1,1-trifluoropropan-2-yl)-4H-1,2,4-triazol-3-yl)pyridin-2-yl)benzamide;
N-(3-(4-cyclopropyl-4H-1,2,4-triazol-3-yl)phenyl)-4-(2-ethylpyrimidin-5-yl)picolinamide;
(R)-4-(4-cyclopropyl-1H-imidazol-1-yl)-N-(3-(4-(1, 1, 1-tri-fluoropropan-2-yl)-4H-1,2,4-triazol-3-yl)phenyl)picolinamide
N-(3-(4-cyclopropyl-4H-1,2,4-triazol-3-yl)phenyl)-5-ethyl-3,4′-bipyridine-2′-carboxamide;
6-cyclopropyl-N-(3-(4-cyclopropyl-4H-1,2,4-triazol-3-yl)-4-fluorophenyl)-3,4′-bipyridine-2′-Carboxamide;
N-(3-(4-cyclopropyl-4H-1,2,4-triazol-3-yl)phenyl)-4(1,5-naphthyridin-3-yl)picolinamide;
N-(6-(4-cyclopropyl-4H-1,2,4-triazol-3-yl)pyridin-2-yl)-3-(1,5-naphthyridin-3-yl)benzamide;
3-(4-cyclopropyl-1H-imidazol-1-yl)-N-(6-(4-cyclopropyl-4H-1,2,4-triazol-3-yl)pyridin-2-yl)benzamide;
N-(3-(4-cyclopropyl-4H-1,2,4-triazol-3-yl)-4-fluorophenyl)-6-ethyl 1-3,4′-bipyridin carboxamide;
6-tert-butyl-N-(3-(4-cyclopropyl-4H-1,2,4-triazol-3-yl)phenyl)-3,4′-bipyridine-2′-carboxamide;
N-(6-(4-cyclopropyl-4H-1,2,4-triazol-3-yl)pyridin-2-yl)-3-(quinolin-3-yl)benzamide;
N-(6-(4-cyclopropyl-4H-1,2,4-triazol-3-yl)pyridin-2-yl)-3-(4-isopropyl-1H-imidazol-1-yl)benzamide;
N-(6-(4-cyclopropyl-4H-1,2,4-triazol-3-yl)pyridin-2-yl)-3-(6-cyclopropylpyridin-3-yl)benzamide;
6-cyclopropyl-N-(2-(4-cyclopropyl-4H-1,2,4-triazol-3-yl)
3-(4-cyclopropyl-1H-imidazol-1-yl)-N-(6-(4-cyclopropyl-pyridin-4-yl)-3,4′-bipyridine-2′-carboxamide;
4H-1,2,4-triazol-3-yl)pyridin-2-yl)-2-methylbenzamide;
N-(6-(4-cyclopropyl-4H-1,2,4-triazol-3-yl)pyridin-2-yl)-4-(4-(trifluoromethyl)-4,5,6,7-tetrahydro-1H-imidazo[4,5-c]pyridin-1-yl)picolinamide;
N-(3-(4-cyclopropyl-4H-1,2,4-triazol-3-yl)phenyl)-4-(4-(trifluoromethyl)-4,5,6, 7-tetrahydro-1H-imidazo[4,5-c]pyridin-1-yl)picolinamide;
5-(4-cyclopropyl-1H-imidazol-1-yl)-N-(6-(4-cyclopropyl-4H-1,2,4-triazol-3-yl)pyridin-2-yl)-2-methylbenzamide;
N-(3-(4-cyclopropyl-4H-1,2,4-triazol-3-yl)phenyl)-4-(4(perfluoroethyl)-1H-imidazol-1-yl)picolinamide;
N-(6-(4-cyclopropyl-4H-1,2,4-triazol-3-yl)pyridin-2-yl)-4-(4-(perfluoroethyl)-1H-imidazol-1-yl)picolinamide;
3-(4-cyclopropyl-1H-imidazol-1-yl)-N-(6-(4-cyclopropyl-4H-1,2,4-triazol-3-yl)pyridin-2-yl)-4-methylbenzamide;
4-(3-cyclopropyl-1H-1,2,4-triazol-1-yl)-N-(6-(4-cyclopropyl-4H-1,2,4-triazol-3-yl)pyridin-2-yl)picolinamide;
4-(3-cyclopropyl-1H-1,2,4-triazol-1-yl)-N-(3-(4-cyclopropyl-4H-1,2,4-triazol-3-yl)phenyl)picolinamide;
4-(5-cyclopropyl-1H-1,2,4-triazol-1-yl)-N-(3-(4-cyclopropyl-4H-1,2,4-triazol-3-yl)phenyl)picolinamide;
N-(6-(4-cyclopropyl-4H-1,2,4-triazol-3-yl)pyridin-2-yl)-3-(6-(2-hydroxypropan-2-yl)pyridin-3-yl)benzamide;
3-(4-cyclopropyl-1H-imidazol-1-yl)-N-(6-(4-cyclopropyl-4H-1,24-triazol-3-yl)pyridin-2-yl)-5-fluoro benzamide;
N-(2-(4-cyclopropyl-4H-1,2,4-triazol-3-yl)pyridin-4-yl)-4-(quinolin-3-yl)picolinamide;
45 N-(3-(4-cyclopropyl-4H-1,2,4-triazol-3-yl)phenyl)-4-(5,6, 7, 8-tetrahydro-1,6-naphthyridin-3-yl)picolinamide;
6-cyclopropyl-N-(3-(4-cyclopropyl-4H-1,2,4-triazol-3-yl)-2-fluorophenyl)-3,4′-bipyridine-2′-carboxamide;
5-cyclopropyl-N-(3-(4-cyclopropyl-4H-1,2,4-triazol-3-yl)phenyl)-3,4′-bipyridine-2′-carboxamide;
N-(3-(4-cyclopropyl-4H-1,2,4-triazol-3-yl)-2-fluorophenyl)-3,4′-bipyridine-2′-carboxamide;
N-(3-(4-cyclopropyl-4H-1,2,4-triazol-3-yl)phenyl)-4-(4-ethyl-1H-imidazol-1-yl)picolinamide;
55 N-(6-(4-cyclopropyl-4H-1,2,4-triazol-3-yl)pyridin-2-yl)-4-(4-methyl-1H-imidazol-1-yl)picolinamide;
N-(6-(4-cyclopropyl-4H-1,2,4-triazol-3-yl)pyridin-2-yl)-4(4,5-dimethyl-1H-imidazol-1-yl)picolinamide;
N-(3-(4-cyclopropyl-4H-1,2,4-triazol-3-yl)phenyl)-4-(6-(2,2,2-trifluoroethyl)-S,6, 7,8-tetrahydro-1,6-naphthyridin-3-yl)picolinamide;
N-(3-(4-cyclopropyl-4H-1,2,4-triazol-3-yl)phenyl)-4-(4-isopropyl-1H-imidazol-1-yl)picolinamide;
N-(3-(4-cyclopropyl-4H-1,2,4-triazol-3-yl)phenyl)-6-(2-hydroxypropan-2-yl)-3,4′-bipyridine-2′-Carboxamide;
N-(6-(4-cyclopropyl-4H-1,2,4-triazol-3-yl)pyridin-2-yl)-6-(2-hydroxypropan-2-yl)-3,4′-bipyridine-2′-Carboxamide;
N-(6-(4-cyclopropyl-4H-1,2,4-triazole-3-yl)pyridin-2-yl)-4-(4-isopropyl-1H-imidazol-1-yl)picolinamide;
6-cyclopropyl-N-(3-(4-cyclopropyl-4H-1,2,4-triazol-3-yl)-5-fluorophenyl)-3,4′-bipyridine-2′-carboxamide;
N-(3-(4-cyclopropyl-4H-1,2,4-triazol-3-yl)-5-fluorophenyl)-3,4′-bipyridine-2′-Carboxamide;
4-(4-cyclopropyl-1H-imidazol-1-yl)-N-(6-(4-cyclopropyl-4H-1,2,4-triazol-3-yl)pyridin-2-yl)picolinamide;
N-(3-(4-cyclopropyl-4H-1,2,4-triazol-3-yl)phenyl)-6-(2,2,2-trifluoroethyl)-3,4′-bipyridine-2′-carboxamide;
N-(3-(4-cyclopropyl-4H-1,2,4-triazol-3-yl)phenyl)-4-(6-isopropyl-5,6, 7,8-tetrahydro-1,6-naphthyridin-3-yl)picolinamide;
N-(3-(4-cyclopropyl-4H-1,2,4-triazol-3-yl)phenyl)-4-(6-methyl-5,6,7,8-tetrahydro-1,6-naphthyridin-3-yl)picolinamide;
N-(3-(4-cyclopropyl-4H-1,2,4-triazol-3-yl)phenyl)-4-(3-hydroxypiperidin-1-yl)picolinamide;
N-(6-(4-cyclopropyl-4H-1,2,4-triazol-3-yl)pyridin-2-yl)-4-(3-hydroxypiperidiN-1-yl)picolinamide;
6-cyclopropyl-N-(6-(4-isopropyl-4H-1,2,4-triazol-3-yl)pyridin-2-yl)-3,4′-bipyridine-2′-carboxamide;
N-(3-(4-cyclopropyl-4H-1,2,4-triazol-3-yl)phenyl)-4-(4-ethyl 1-3-oxopiperaziN-1-yl)picolinamide;
N-(6-(4-cyclopropyl-4H-1,2,4-triazol-3-yl)pyridin-2-yl)-4-(4-ethyl-3-oxopiperaziN-1-yl)picolinamide;
(R)-6-cyclopropyl-N-(6-(4-(1,1,1-trifluoropropan-2-yl)-4H-1,2,4-triazol-3-yl)pyridin-2-yl)-3,4′-bipyridine-2′-carboxamide;
N-(3-(4-isopropyl-4H-1,2,4-triazol-3-yl)phenyl)-3,4′-bipyridine-2′-carboxamide;
6-cyclopentyl-N-(3-(4-cyclopropyl-4H-1,2,4-triazol-3-yl)phenyl)-3,4′-bipyridine-2′-carboxamide;
N-(3-(4-cyclopropyl-4H-1,2,4-triazol-3-yl)phenyl)-6-(1-methyl-2-oxopyrrolidin-3-yl)-3,4′-bipyridine-2′-Carboxamide;
N-(6-(4-cyclopropyl-4H-1,2,4-triazol-3-yl)pyridin-2-yl)-4-(4-(N-methyl sulfamoyl)phenyl)picolinamide;
N-(6-(4-cyclopropyl-4H-1,2,4-triazol-3-yl)pyridin-2-yl)-4-(quinolin-3-yl)picolinamide;
N-(6-(4-cyclopropyl-4H-1,2,4-triazol-3-yl)pyridin-2-yl)-4-(4-phenyl-1H-imidazol-1-yl)picolinamide;
N-(3-(4-cyclopropyl-4H-1,2,4-triazol-3-yl)phenyl)-6-propyl-3,4′-bipyridine-2′-Carboxamide;
N-(3-(4-cyclopropyl-4H-1,2,4-triazol-3-yl)phenyl)-6-neopentyl-3,4′-bipyridine-2′-Carboxamide;
N-(3-(4-cyclopropyl-4H-1,2,4-triazol-3-yl)phenyl)-4-(1-methyl-2-phenyl-1H-imidazol-5-yl)picolinamide;
N-(3-(4-cyclopropyl-4H-1,2,4-triazol-3-yl)phenyl)-4-(4-(ethyl sulfonyl)phenyl)-picolinamide;
N-(3-(4-cyclopropyl-4H-1,2,4-triazol-3-yl)phenyl)-4-(4-(isopropylsulfonyl)phenyl)picolinamide;
N-(3-(4-cyclopropyl-4H-1,2,4-triazol-3-yl)phenyl)-6-(ethylamino)-3,4′-bipyridine-2′-carboxamide;
N-(3-(4-cyclopropyl-4H-1,2,4-triazol-3-yl)phenyl)-6-(cyclopropylamino)-3,4′-bipyridine-2′-carboxamide;
N-(3-(4-cyclopropyl-4H-1,2,4-triazol-3-yl)phenyl)-4-(imidazo[2,1-b] [1,3,4]thiadiazol-5-yl)picolinamide;
4-(4-chloro-1H-imidazol-1-yl)-N-(3-(4-cyclopropyl-4H-1,2,4-triazol-3-yl)phenyl)picolinamide;
N-(3-(4-cyclopropyl-4H-1,2,4-triazol-3-yl)phenyl)-4-(2-cyclopropylpyrimidin-5-yl)picolinamide;
N-(3-(4-cyclopropyl-4H-1,2,4-triazol-3-yl)phenyl)-6′-(trifluoromethyl)-3,4′-bipyridine-2′-carboxamide;
N-(3-(4-cyclopropyl-4H-1,2,4-triazol-3-yl)phenyl)-4-(quinolin-3-yl)-6-(trifluoromethyl)picolinamide;
N-(6-(1-cyclopropyl-1H-imidazol-5-yl)pyridin-2-yl)-4-(quinolin-3-yl)picolinamide;
6-cyclopropyl-N-(6-(1-cyclopropyl-1H-imidazol-5-yl)pyridin-2-yl)-3,4′-bipyridine-2′-carboxamide;
5 N-(3-(4-cyclopropyl-4H-1,2,4-triazol-3-yl)phenyl)-4-(1Hpyrrolo[3,2-b]pyridin-6-yl)picolinamide;
N-(3-(4-cyclopropyl-4H-1,2,4-triazol-3-yl)phenyl)-4-(4-cyclopropylphenyl)picolinamide;
N-(6-(4-cyclopropyl-4H-1,2,4-triazol-3-yl)pyridin-2-yl)-3-10 (pyridin-3-yl)benzamide;
N-(3-(4-cyclopropyl-4H-1,2,4-triazol-3-yl)phenyl)-6-(methylthio)-3,4′-bipyridine-2′-carboxamide;
N-(3-(4-cyclopropyl-4H-1,2,4-triazol-3-yl)phenyl)-6-(isobutylthio)-3,4′-bipyridine-2′-carboxamide;
15 N-(3-(4-cyclopropyl-4H-1,2,4-triazol-3-yl)phenyl)-4-(5-cyclopropylpyrazin-2-yl)picolinamide;
6-cyclopropyl-N-(3-(4-cyclopropyl-4H-1,2,4-triazol-3-yl)phenyl)-5-fluoro-3,4′-bipyridine-2′-carboxamide;
5-chloro-6-cyclopropyl-N-(3-(4-cyclopropyl-4H-1,2,4-triazol-3-yl)phenyl)-3,4′-bipyridine-2′-carboxamide;
N-(3-(4-cyclopropyl-4H-1,2,4-triazol-3-yl)phenyl)-6-(2-methoxyethylamino)-3,4′-hi pyridine-2′-carboxamide;
N-(3-(4-cyclopropyl-4H-1,2,4-triazol-3-yl)phenyl)-4-(4-(methylsulfonyl)piperazin-1-yl)picolinamide;
N-(3-(4-cyclopropyl-4H-1,2,4-triazol-3-yl)phenyl)-6-ethyl-5-fluoro-3,4′-bipyridine-2′-carboxamide;
5-chloro-N-(3-(4-cyclopropyl-4H-1,2,4-triazol-3-yl)phenyl)-6-ethyl-3,4′-bipyridine-2′-Carboxamide;
4-(4-cyclopropyl-1H-imidazol-1-yl)-N-(3-(4-cyclopropyl-4H-1,2,4-triazol-3-yl)phenyl)picolinamide;
N-(3-(4-cyclopropyl-4H-1,2,4-triazol-3-yl)phenyl)-5,6-diethyl-3,4′-bipyridine-2′-carboxamide;
N-(3-(4-cyclopropyl-4H-1,2,4-triazol-3-yl)phenyl)-4-(furo[3,2-b]pyridin-6-yl)picolinamide;
N-(4-cyclopropyl-4H-1,2,4-triazol-3-yl)phenyl)-4-(3-methyl-3H-imidazo[4,5-b]pyridin-6-yl)picolinamide;
N-(3-(4-cyclopropyl-4H-1,2,4-triazol-3-yl)phenyl)-6-(6-cyclopropylpyridin-3-yl)pyrimidine-4-carboxamide.
6-cyclopropyl-N-(3-(4-cyclopropyl-4H-1,2,4-triazol-3-yl)phenyl)-6′-methyl-3,4′-bipyridine-2′-carboxamide;
6-cyclopropyl-N-(3-(4-cyclopropyl-4H-1,2,4-triazol-3-yl)phenyl)-5′-methyl-3,4′-bipyridine-2′-carboxamide;
N-(6-(4-cyclopropyl-4H-1,2,4-triazol-3-yl)pyridin-2-yl)-4-(5-methyl-4-(trifluoromethyl)-4,5,6,7-tetrahydro-1H-imidazo[4,5-c]pyridin-1-yl)picolinamide;
N-(3-(4-cyclopropyl-4H-1,2,4-triazol-3-yl)phenyl)-4-(5-methyl-4-(trifluoromethyl)-4,5,6, 7-tetrahydro-1H-imidazo[4,5-c]pyridin-1-yl)picolinamide;
6′-cyclopropyl-N-(6-(4-cyclopropyl-4H-1,2,4-triazol-3-yl)pyridin-2-yl)-2,31-bipyridine-6-carboxamide;
6′-cyclopropyl-N-(6-(4-cyclopropyl-4H-1,2,4-triazol-3-yl)pyridin-2-yl)-3,31-bipyridine-5-carboxamide;
6′-cyclopropyl-N-(6-(4-cyclopropyl-4H-1,2,4-triazol-3-yl)pyridin-2-yl)-2,31-bipyridine-4-carboxamide;
N-(6-(4-cyclopropyl-4H-1,2,4-triazol-3-yl)pyridin-2-yl)-5-(6-cyclopropylpyridin-3-yl)-2,4-difluorobenzamide;
6′-cyclopropyl-N-(3-(4-cyclopropyl-4H-1,2,4-triazol-3-yl)phenyl)-2,31-bipyridine-6-carboxamide;
(S)-4-(4-cyclopropyl-1H-imidazol-1-yl)-N-(3-(4-(3-methylbutan-2-yl)-4H-1,2,4-triazol-3-yl)phenyl)picolinamide;
4-chloro-N-(6-(4-cyclopropyl-4H-1,2,4-triazol-3-yl)pyridin-2-yl)-5-(6-cyclopropylpyridin-3-yl)-2-fluorobenzamide;
6-cyclopropyl-N-(3-(4-(2-phenylcyclopropyl)-4H-1,2,4-triazol-3-yl)phenyl)-3,4′-bipyridine-2′-carboxamide;
N-(3-(4-cyclopropyl-4H-1,2,4-triazol-3-yl)phenyl)-6-(cyclopropylmethyl)-3,4′-bipyridine-2′-carboxamide;
3-(4-cyclopropyl-1H-1,2,3-triazol-1-yl)-N-(6-(4-cyclopropyl-4H-1,2,4-triazol-3-yl)pyridin-2-yl)benzamide;
4-(5-cyclopropyl-1,3,4-thiadiazol-2-yl)-N-(3-(4-cyclopropyl-4H-1,2,4-triazol-3-yl)phenyl)picolinamide;
6-cyclopropyl-N-(3-(4-phenyl-4H-1,2,4-triazol-3-yl)phenyl)-3,4′-bipyridine-2′-carboxamide;
6-cyclopropyl-N-(3-(4-(pyridin-2-yl)-4H-1,2,4-triazol-3-yl) phenyl)-3,4′-bipyridine-2′-carboxamide;
6-cyclopropyl-N-(3-(4-(pyridin-3-yl)-4H-1,2,4-triazol-3-yl)phenyl)-3,4′-bipyridine-2′-carboxamide;
6-cyclopropyl-N-(3-(4-(pyridin-4-yl)-4H-1,2,4-triazol-3-yl)phenyl)-3,4′-bipyridine-2′-carboxamide;
6-cyclopropyl-N-(3-(4-(pyrimidin-5-yl)-4H-1,2,4-triazol-3-yl)phenyl)-3,4′-bipyridine-2′-carboxamide;
4-(4-cyclopropyl-1H-imidazol-1-yl)-N-(3-(4-(pyridin-3-yl)-4H-1,2,4-triazol-3-yl)phenyl)picolinamide;
4-(4-cyclopropyl-1H-imidazol-1-yl)-N-(3-(4-(pyridin-4-yl)-4H-1,2,4-triazol-3-yl)phenyl)picolinamide;
4-(4-cyclopropyl-1H-imidazol-1-yl)-N-(3-(4-(pyrimidin-5-yl)-4H-1,2,4-triazol-3-yl)phenyl)picolinamide; and
N-(3-(4-(but-2-ynyl)-4H-1,2,4-triazol-3-yl)phenyl)-4-(4-cyclopropyl-1H-imidazol-1-yl)picolinamide; or a pharmaceutically acceptable salt or solvate thereof.

In some embodiments, the ASK1 inhibiting compound is a compound of the structure:

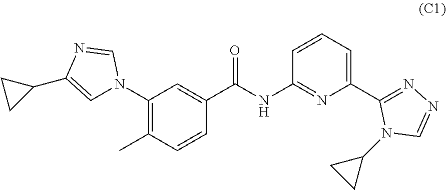 [SMILES: Cc1ccc(C(=O)Nc2cccc(-c3nncn3C3CC3)n2)cc1-n1cnc(C2CC2)c1]

or a pharmaceutically acceptable salt or hydrate thereof. This compound may be referred to as 3-(4-cyclopropyl-1H-imidazol-1-yl)-N-(6-(4-cyclopropyl-4H-1,2,4-triazol-3-yl)pyridin-2-yl)-4-methylbenzamide or 3-(4-cyclopropyl-1H-imidazol-1-yl)-N-[6-(4-cyclopropyl-4H-1,2,4-triazol-3-yl)-2-pyridinyl]-4-methylbenzamide, and has been assigned CAS Registry No. 1262041-67-7. The compound and salts thereof including formic acid salt (CAS Reg. No. 1262041-68-8) may be prepared by methods disclosed in US 2014/0228412 and U.S. Pat. No. 9,067,933.

In other embodiments, the ASK1 inhibiting compound is a compound of the structure:

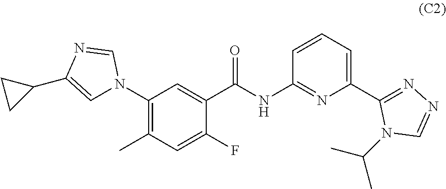 [SMILES: Cc1cc(F)c(C(=O)Nc2cccc(-c3nncn3C(C)C)n2)cc1-n1cnc(C2CC2)c1]

or a pharmaceutically acceptable salt or hydrate thereof. This compound may be referred to as 5-(4-cyclopropyl-1H-imidazol-1-yl)-2-fluoro-N-(6-(4-isopropyl-4H-1,2,4-triazol-3-yl)pyridin-2-yl)-4-methylbenzamide or 5-(4-cyclopropyl-1H-imidazol-1-yl)-2-fluoro-4-methyl-N-[6-[4-(1-methylethyl)-4H-1,2,4-triazol-3-yl]-2-pyridinyl]-benzamide, and has been assigned CAS Registry No. 1448428-04-3. The compound and salts thereof, including hydrochloride salt (CAS Reg. No. 1448428-05-4) may be prepared by methods disclosed in US 2014/0228412 and U.S. Pat. No. 9,067,933.

It will be understood that the terms “inhibitor”, “inhibiting compound”, and the like, refer to a compound or agent which presents a pharmaceutical activity to inhibit activity of certain target in a subject such as human. For example, it will be understood that the terms “ASK1 inhibitor”, “ASK1 inhibiting compound”, and “inhibitor of ASK1”, and the like, refer to compounds which present a pharmaceutical activity to inhibit activity of an apoptosis signal-regulating kinase 1 in a human. In some embodiments of each of the methods herein, Compound C2 or a pharmaceutically acceptable salt thereof, is used in combination with Compound A1, or a pharmaceutically acceptable salt or hydrate thereof. In other embodiments of each of the methods herein, a compound of Formula I, or a pharmaceutically acceptable salt thereof, is used in combination with Compound A1, or a pharmaceutically acceptable salt or hydrate thereof. In another variation, the ASK1 inhibiting compound is 4-[4-[(4′-chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl] benzamide, or a pharmaceutically acceptable salt thereof.

### Bromodomain Inhibitors

In some variations the BET or BRD (bromodomain-containing protein) inhibitor is an inhibitor of bromodomain-containing protein 4 (BRD4). In one aspect the modulator of a bromodomain-containing protein is a compound of Formula (II):

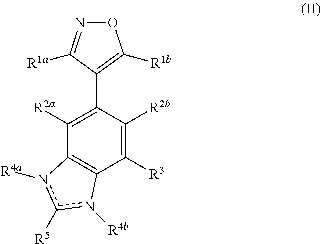

wherein

R^{1a} and R^{1b} are each independently C₁₋₆ alkyl optionally substituted with from 1 to 5 R²⁰ groups;

R^{2a} and R^{2b} are each independently H or halo;

R³ is

C(O)OR^{a}, —NHC(O)OR^{a}, —NHS(O)₂R^{a}, or —S(O)₂NR^{a}R^{b}; or

selected from the group consisting of C₁₋₁₀ alkyl, C₁₋₁₀ alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups;

one of R^{4a} and R^{4b} is selected from the group consisting of H and C₁₋₆ alkyl optionally substituted with from 1 to 5 R20 groups, and the other is absent;

R⁵ is —C(O)OR^{a}, —NHC(O)OR^{a}, —NHS(O)₂R^{a}, or —S(O)₂NR^{a}R^{b}; or

R⁵ is selected from the group consisting of H, C₁₋₁₀ alkyl, C₁₋₁₀ haloalkyl, C₁₋₁₀ alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups;

each R^{a} and R^{b} is independently selected from the group consisting of H, C₁₋₁₀ alkyl, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups; and

each R²⁰ is independently selected from the group consisting of acyl, C₁₋₁₀ alkyl, C₁₋₁₀ alkoxy, amino, amido, amidino, C₅₋₁₀ aryl, C₆₋₂₀ arylalkyl, azido, carbamoyl, carboxyl, carboxyl ester, cyano, guanidino, halo, C₁₋₁₀ haloalkyl, C₁₋₁₀ heteroalkyl, C₅₋₁₀ heteroaryl, C₆₋₂₀ heteroarylalkyl, hydroxy, hydrazino, imino, oxo, nitro, sulfinyl, sulfonic acid, sulfonyl, thiocyanate, thiol, and thione;

wherein the C₁₋₁₀ alkyl, C₅₋₁₀ aryl, C₆₋₂₀ arylalkyl, C₁₋₁₀ heteralkyl, C₅₋₁₀ heteroaryl, and C₆₋₂₀ heteroarylalkyl groups are optionally substituted with from 1 to 3 substituents independently selected from C₁₋₆ alkyl, C₅₋₁₀ aryl, halo, C₁₋₆ haloalkyl, cyano, hydroxy, and C₁₋₆ alkoxy;

or a pharmaceutically acceptable salt thereof.

Compounds of Formula (II) (which include compounds of any of Formulae (IIa), (IIb), (IIc), (IId) and (IIe), described below) can include, independently, one or more of the following features. It will be recognized that features specified in each embodiment may be combined with other specified features to provide further embodiments.

In some compounds, R^{1a} and R^{1b} are each independently C₁₋₆ alkyl which, as defined herein, includes alkenyl, alkynyl and cycloalkyl. In some compounds, R^{1a} and R^{1b} are different, and in other compounds R^{a1} and R^{1b} are the same. In some compounds, R^{1a} and R^{1b} are each independently a C₁₋₆ alkyl optionally substituted with 1-5 R²⁰ groups. In some compounds, R₁ₐ and R^{1b} are both methyl. In some compounds, one of R^{1a} or R^{1b} is a methyl and the other is a methyl substituted with a hydroxy. In some compounds, R^{1a} and R^{1b} are both methyl substituted with a hydroxy. In some compounds, one of R^{1a} or R^{1b} is a methyl and the other is a methyl substituted with an amine. In some compounds, R^{1a} and R^{1b} are both methyl substituted with an amine.

In some compounds, R^{2a} and R^{2b} are both H. In some compounds, R^{2a} and R^{2b} are both halo. In some compounds, one of R^{2a} and R^{2b} is H and the other is halo. In some compounds the halo is —F or -Cl.

In some compounds, R³ is boronic acid, a boronic acid ester, or halo. In some compounds, R³ is —C(O)OR^{a}, —NHC(O)OR^{a}, —NHS(O)₂R^{a}, or —S(O)₂NR^{a}R^{b} wherein R^{a} and R^{b} are described above. In some compounds, R is —C(O)OR^{a}, —NHC(O)OR^{a}, —NHS(O)₂R^{a}, or —S(O)₂NR^{a}R^{b}, wherein each R^{a} and R^{b} is independently C₁₋₁₀ alkyl, C₅₋₁₀ aryl, C₁₋₁₀ heteroalkyl or C₅₋₁₀ heteroaryl, each of which may be optionally substituted as described above. For example, in some compounds R³ is —C(O)OR^{a}, —NHC(O)OR^{a}, —NHS(O)₂R^{a}, or —S(O)₂NR^{a}R^{b}, wherein each R^{a} and R^{b} is independently C₅₋₁₀ aryl or C₅₋₁₀ heteroaryl. In some compounds, R³ is selected from the group consisting of C₁₋₁₀ alkyl, C₁₋₁₀ alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups, wherein R²⁰ is described above. In some compounds, R³ is C₁₋₁₀ alkyl, C₁₋₁₀ alkoxy, or C₁₋₁₀ heteroalkyl, each of which may be optionally substituted as described above. In some compounds, the heteroalkyl is a heterocycloalkyl. In other compounds, R³ is C₆₋₂₀ arylalkyl or C₆₋₂₀ heteroarylalkyl, each of which may be optionally substituted as described above. In other compounds, R³ is C₅₋₁₀ aryl, C₆₋₂₀ arylalkyl, C₅₋₁₀ heteroaryl, or C₆₋₂₀ heteroarylalkyl, each of which may be optionally substituted as described above. In some compounds, R³ is amino optionally substituted as described above. For example, in some compounds R³ is —NH₂, and in other compounds R³ is —NR^{y}R^{z}, wherein R^{y} and R^{z} together with the nitrogen to which they are bonded form a C₁₋₁₀ heteroalkyl or C₅₋₁₀ heteroaryl, each of which may be optionally substituted as described above.

Other non-limiting examples of R³ include the following:

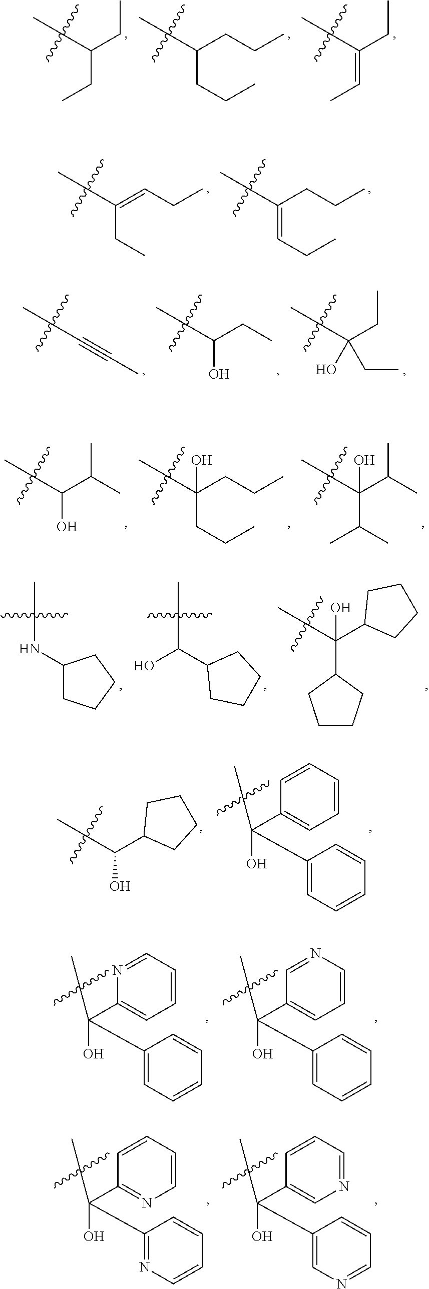

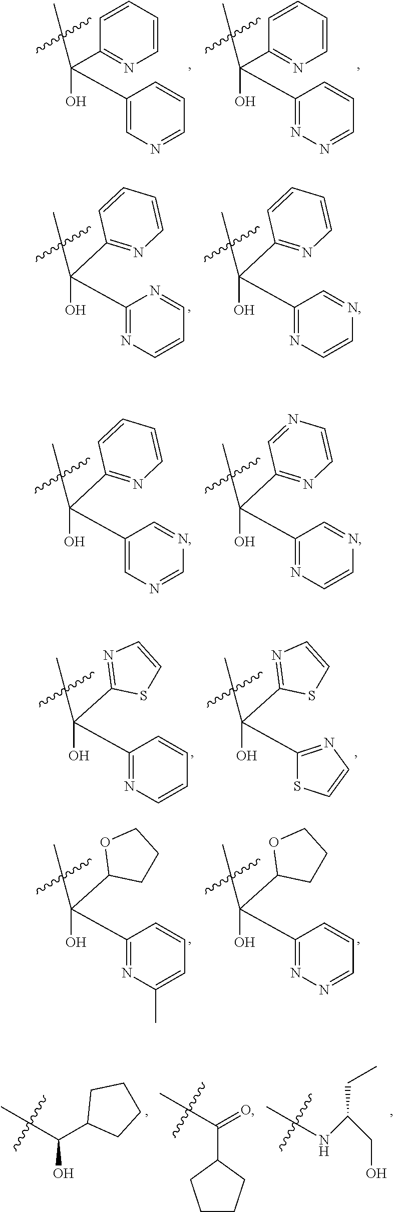

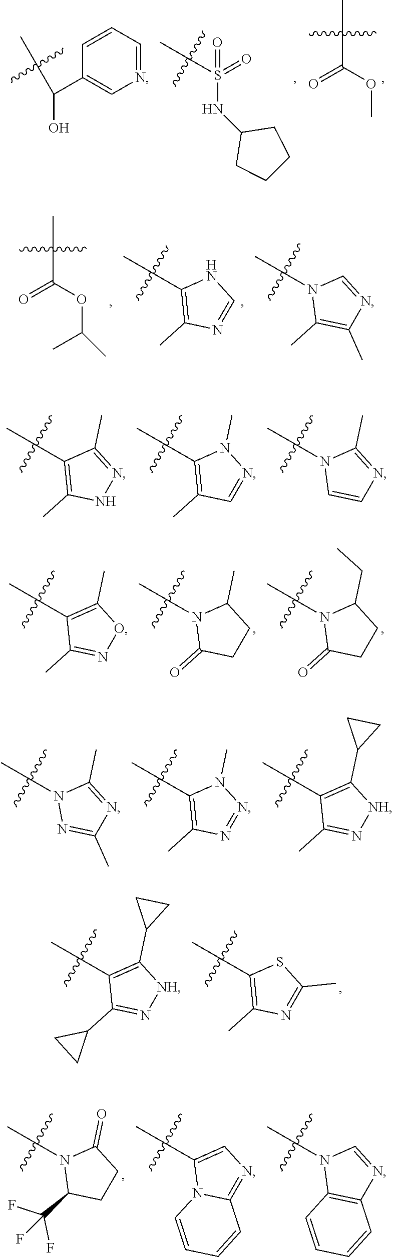

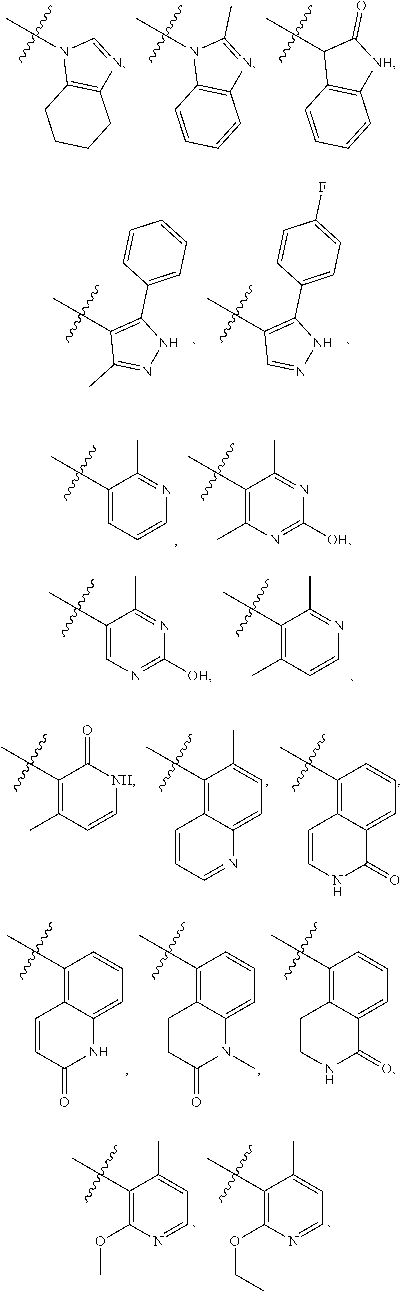

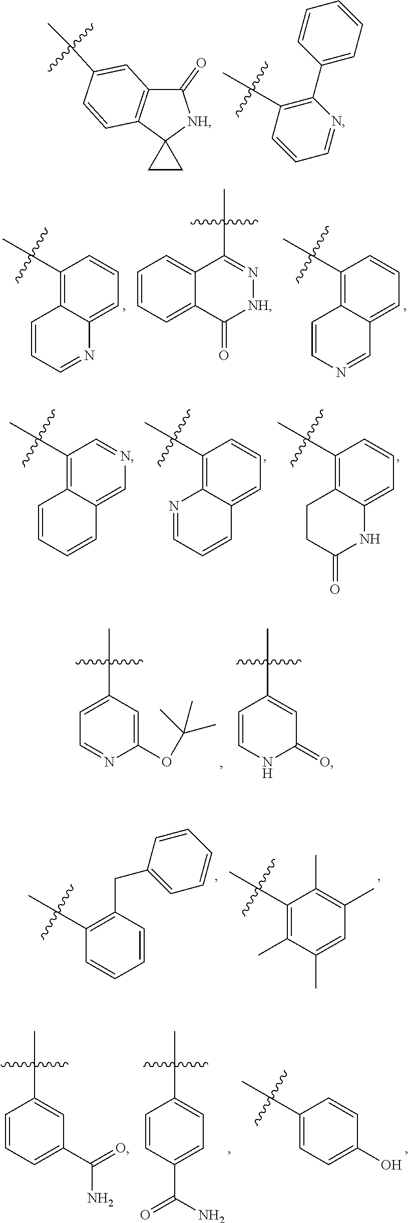

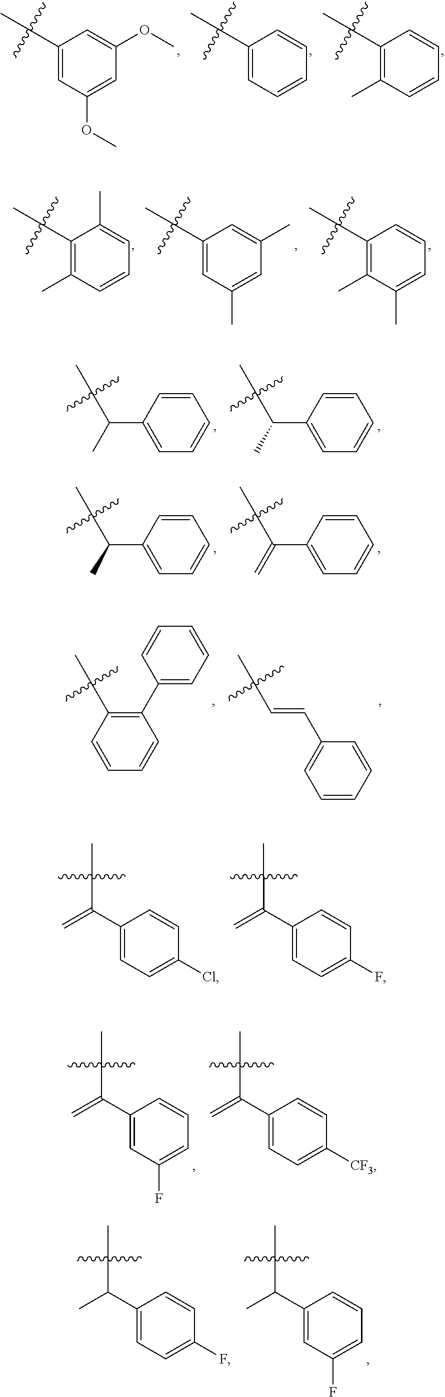

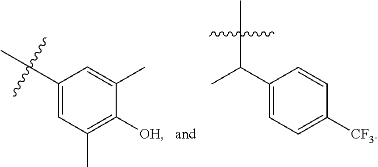

In some compounds, one of R^{4a} or R^{4b} is H and the other is absent, that is, in some compounds R^{4a} is H and R^{4b} is absent, and in other compounds R^{4a} is absent and R^{4b} is H. In other compounds, one of R^{4a} and R^{4b} is alkyl and the other is absent, that is, in some compounds R^{4a} is alkyl and R^{4b} is absent, and in other compounds R^{4a} is absent and R^{4b} is alkyl. In some compounds the alkyl is methyl.

In some compounds, R⁵ is —C(O)OR^{a}, —NHC(O)OR^{a}, —NHS(O)₂R^{a}, or -S(O)₂NR^{a}R^{b}, wherein R^{a} and R^{b} are described above. In some compounds, R⁵ is —C(O)OR^{a}, —NHC(O)OR^{a}, —NHS(O)₂R^{a}, or —S(O)₂NR^{a}R^{b}, wherein each R^{a} and R^{b} is independently C₁₋₁₀ alkyl or C₅₋₁₀ aryl, each of which may be optionally substituted as described above. For example, in some compounds R⁵ is —NHC(O)OR^{a}, wherein R^{a} is methyl. In some compounds, R⁵ is —NHS(O)₂R^{a}, wherein R^{a} is C₁₋₁₀ alkyl or C₅₋₁₀ aryl, each of which may be optionally substituted as described above. For example, in some compounds R⁵ is —NHS(O)₂R^{a}, wherein R^{a} is cyclopropyl. In some compounds, R⁵ is selected from the group consisting of H, C₁₋₁₀ alkyl, C₁₋₁₀ haloalkyl, C₁₋₁₀ to alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups, wherein R²⁰ is described above. In some compounds, R⁵ is C₁₋₁₀ alkyl optionally substituted as described above. In some compounds the C₁₋₁₀ alkyl is a C₁₋₁₀ cycloalkyl, e.g. cyclopropyl. In other compounds, R⁵ is amino optionally substituted as described above. For example, in some compounds R⁵ is —NH₂, and in other compounds R⁵ is —NR^{y}R^{z}, wherein R^{y} is H and R^{z} is alkyl, e.g. cyclopropyl. In other compounds, R⁵ is alkoxy, e.g. methoxy.

In some compounds, R^{1a}, R^{1b}, R³, R^{4a}, R^{4b} and R⁵ are optionally substituted with from 1 to 5 (i.e. 1, 2, 3, 4 or 5) R²⁰ groups as described above. In some compounds, R^{1a}, R^{1b}, R³, R^{4a}, R^{4b} and R⁵ are optionally substituted with 1, 2, or 3 R²⁰ groups. In some compounds, each R²⁰ is independently selected from the group consisting of alkyl, alkoxy, amino, cyano, halo, haloalkyl, heteroalkyl, hydroxy, and sulfonyl. In some compounds, each R is independently selected from the group consisting of aryl, alkylaryl, heteroaryl, and heteroalkylaryl. In some compounds, R^{1a}, R^{1b}, R³, R^{4a}, R^{4b} and R⁵ are not substituted. In some compounds, R²⁰ is not substituted.

One subset of compounds of Formula (II) relates to compounds of Formula (IIa)

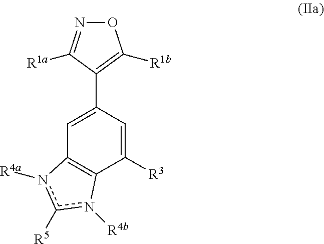

wherein

R^{1a} and R^{1b} are each independently C₁₋₆ alkyl optionally substituted with from 1 to 5 R²⁰ groups;

R³ is

boronic acid or halo; or

C(O)OR^{a}, —NHC(O)OR^{a}, —NHS(O)₂R^{a}, or —S(O)₂NR^{a}R^{b}; or

selected from the group consisting of C₁₋₁₀ alkyl, C₁₋₁₀ alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups;

one of R^{4a} and R^{4b} is selected from the group consisting of H and C₁₋₆ alkyl optionally substituted with from 1 to 5 R²⁰ groups, and the other is absent;

R⁵ is

C(O)OR^{a}, —NHC(O)OR^{a}, —NHS(O)₂R^{a}, or —S(O)₂NR^{a}R^{b}; or

selected from the group consisting of H, C₁₋₁₀ alkyl, C₁₋₁₀ haloalkyl, C₁₋₁₀ alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups;
each R^{a} and R^{b} is independently selected from the group consisting of H, C₁₋₁₀ alkyl, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups; and

each R²⁰ is independently selected from the group consisting of acyl, C₁₋₁₀ alkyl, C₁₋₁₀ alkoxy, amino, amido, amidino, C₅₋₁₀ aryl, C₆₋₂₀ arylalkyl, azido, carbamoyl, carboxyl, carboxyl ester, cyano, guanidino, halo, C₁₋₁₀ haloalkyl, C₁₋₁₀ heteroalkyl, C₅₋₁₀ heteroaryl, C₆₋₂₀ heteroarylalkyl, hydroxy, hydrazino, imino, oxo, nitro, sulfinyl, sulfonic acid, sulfonyl, thiocyanate, thiol, and thione;

wherein the C₁₋₁₀ alkyl, C₅₋₁₀ aryl, C₆₋₂₀ arylalkyl, C₁₋₁₀ heteroalkyl, C₅₋₁₀ heteroaryl, and C₆₋₂₀ heteroarylalkyl groups are optionally substituted with from 1 to 3 substituents independently selected from C₁₋₆ alkyl, C₅₋₁₀ aryl, halo, C₁₋₆ haloalkyl, cyano, hydroxy, and C₁₋₆ alkoxy;

or a pharmaceutically acceptable salt thereof.

Another subset of compounds of Formula (II) relates to compounds of Formula (IIb)

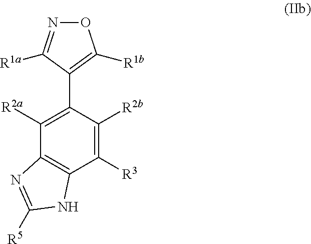

wherein

R^{1a} and R^{1b} are each independently C₁₋₆ alkyl optionally substituted with from 1 to 5 R²⁰ groups;

R^{2a} and R^{2b} are each independently H or halo;

R³ is

boronic acid or halo; or

C(O)OR^{a}, —NHC(O)OR^{a}, —NHS(O)₂R^{a}, or —S(O)₂NR^{a}R^{b}; or

selected from the group consisting of C₁₋₁₀ alkyl, C₁₋₁₀ alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups;

R⁵ is

C(O)OR^{a}, —NHC(O)OR^{a}, —NHS(O)₂R^{a}, or —S(O)₂NR^{a}R^{b}; or

selected from the group consisting of H, C₁₋₁₀ alkyl, C₁₋₁₀ haloalkyl, C₁₋₁₀ alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups;

each R^{a} and R^{b} is independently selected from the group consisting of H, C₁₋₁₀ alkyl, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups; and

each R²⁰ is independently selected from the group consisting of acyl, C₁₋₁₀ alkyl, C₁₋₁₀ alkoxy, amino, amido, amidino, C₅₋₁₀ aryl, C₆₋₂₀ arylalkyl, azido, carbamoyl, carboxyl, carboxyl ester, cyano, guanidino, halo, C₁₋₁₀ haloalkyl, C₁₋₁₀heteroalkyl, C₅₋₁₀ heteroaryl, C₆₋₂₀ heteroarylalkyl, hydroxy, hydrazino, imino, oxo, nitro, sulfinyl, sulfonic acid, sulfonyl, thiocyanate, thiol, and thione;

wherein the C₁₋₁₀ alkyl, C₅₋₁₀ aryl, C₆₋₂₀ arylalkyl, C₁₋₁₀ heteroalkyl, C₅₋₁₀ heteroaryl, and C₆₋₂₀ heteroarylalkyl groups are optionally substituted with from 1 to 3 substituents independently selected from C₁₋₆ alkyl, C₅₋₁₀ aryl, halo, C₁₋₆ haloalkyl, cyano, hydroxy, and C₁₋₆ alkoxy;

or a pharmaceutically acceptable salt thereof.

Another subset of compounds of Formula (II) relates to compounds of Formula (IIc)

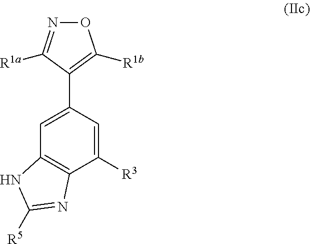

wherein

R^{1a} and R^{1b} are each independently C₁₋₆ alkyl optionally substituted with from 1 to 5 R²⁰ groups;

R³ is

boronic acid or halo; or

C(O)OR^{a}, —NHC(O)ORₐ, —NHS(O)₂R^{a}, or —S(O)₂NRₐR^{b}; or

selected from the group consisting of C₁₋₁₀ alkyl, C₁₋₁₀ alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups;

R⁵ is

C(O)OR^{a}, —NHC(O)OR^{a}, —NHS(O)₂R^{a}, or —S(O)₂NR^{a}R^{b}; or

selected from the group consisting of H, C₁₋₁₀ alkyl, C₁₋₁₀ haloalkyl, C₁₋₁₀ alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups;

each R^{a} and R^{b} is independently selected from the group consisting of H, C₁₋₁₀ alkyl, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups; and

each R²⁰ is independently selected from the group consisting of acyl, C₁₋₁₀ alkyl, C₁₋₁₀ alkoxy, amino, amido, amidino, C₅₋₁₀ aryl C₆₋₂₀ arylalkyl, azido, carbamoyl, carboxyl, carboxyl ester, cyano, guanidino, halo, C₁₋₁₀ haloalkyl, C₁₋₁₀ heteroalkyl, C₅₋₁₀ heteroaryl, C₆₋₂₀ heteroarylalkyl, hydroxy, hydrazino, imino, oxo, nitro, sulfinyl, sulfonic acid, sulfonyl, thiocyanate, thiol, and thione;

wherein the C₁₋₁₀ alkyl, C₅₋₁₀ aryl, C₆₋₂₀ arylalkyl, C₁₋₁₀ heteroalkyl, C₅₋₁₀ heteroaryl, and C₆₋₂₀ heteroarylalkyl groups are optionally substituted with from 1 to 3 substituents independently selected from C₁₋₆ alkyl, C₅₋₁₀ aryl, halo, C₁₋₆ haloalkyl, cyano, hydroxy, and C₁₋₆ alkoxy;

or a pharmaceutically acceptable salt thereof.

Another subset of compounds of Formula (II) relates to compounds of Formula (IId)

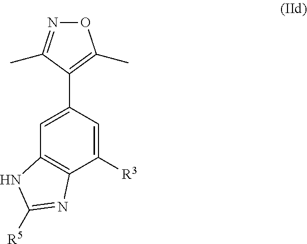

wherein

R³ is

boronic acid or halo; or

C(O)OR^{a}, —NHC(O)OR^{a}, —NHS(O)₂R^{a}, or —S(O)₂NR^{a}R^{b}; or

selected from the group consisting of C₁₋₁₀ alkyl, C₁₋₁₀ alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups;

R⁵ is

C(O)OR^{a}, —NHC(O)OR^{a}, —NHS(O)₂R^{a}, or —S(O)₂NR^{a}R^{b}; or

selected from the group consisting of H, C₁₋₁₀ alkyl, C₁₋₁₀ haloalkyl, C₁₋₁₀ alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups;

each R^{a} and R^{b} is independently selected from the group consisting of H, C₁₋₁₀ alkyl, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups; and

each R²⁰ is independently selected from the group consisting of acyl, C₁₋₁₀ alkyl, C₁₋₁₀ alkoxy, amino, amido, amidino, C₅₋₁₀ aryl, C₆₋₂₀ arylalkyl, azido, carbamoyl, carboxyl, carboxyl ester, cyano, guanidino, halo, C₁₋₁₀ haloalkyl, C₁₋₁₀ heteroalkyl, C₅₋₁₀ heteroaryl, C₆₋₂₀ heteroarylalkyl, hydroxy, hydrazino, imino, oxo, nitro, sulfinyl, sulfonic acid, sulfonyl, thiocyanate, thiol, and thione;

wherein the C₁₋₁₀ alkyl, C₅₋₁₀ aryl, C₆₋₂₀ arylalkyl, C₁₋₁₀ heteroalkyl, C₅₋₁₀ heteroaryl, and C₆₋₂₀ heteroarylalkyl groups are optionally substituted with from 1 to 3 substituents independently selected from C₁₋₆ alkyl, C₅₋₁₀ aryl, halo, C₁₋₆ haloalkyl, cyano, hydroxy, and C₁₋₆ alkoxy;

or a pharmaceutically acceptable salt thereof.

Another subset of compounds of Formula (II) relates to compounds of Formula (IIe)

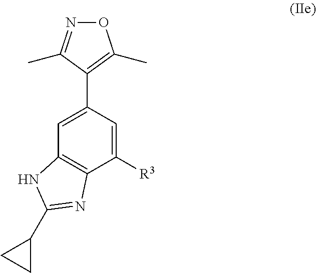

wherein

R³ is

boronic acid or halo; or

C(O)OR^{a}, —NHC(O)OR^{a}, —NHS(O)₂R^{a}, or —S(O)₂NR^{a}R^{b}; or

selected from the group consisting of C₁₋₁₀ alkyl, C₁₋₁₀ alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups;

each R^{a} and R^{b} is independently selected from the group consisting of H, C₁₋₁₀ alkyl, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups; and

each R²⁰ is independently selected from the group consisting of acyl, C₁₋₁₀ alkyl, C₁₋₁₀ alkoxy, amino, amido, amidino, C₅₋₁₀ aryl, C₆₋₂₀ arylalkyl, azido, carbamoyl, carboxyl, carboxyl ester, cyano, guanidino, halo, C₁₋₁₀ haloalkyl, C₁₋₁₀ heteroalkyl, C₅₋₁₀ heteroaryl, C₆₋₂₀ heteroarylalkyl, hydroxy, hydrazino, imino, oxo, nitro, sulfinyl, sulfonic acid, sulfonyl, thiocyanate, thiol, and thione;

wherein the C₁₋₁₀ alkyl, C₅₋₁₀ aryl, C₆₋₂₀ arylalkyl, C₁₋₁₀ heteroalkyl, C₅₋₁₀ heteroaryl, and C₆₋₂₀ heteroarylalkyl groups are optionally substituted with from 1 to 3 substituents independently selected from C₁₋₆ alkyl, C₅₋₁₀ aryl, halo, C₁₋₆ haloalkyl, cyano, hydroxy, and C₁₋₆ alkoxy;

or a pharmaceutically acceptable salt thereof.

In separate embodiments within each of the compounds described for Formulas II, IIa, IIb, and IIc, there is another embodiment comprising a compound in which R^{1a} and R^{1b} are each independently C₁₋₆ alkyl, or a pharmaceutically acceptable salt thereof. In separate embodiments within each of the compounds described for Formulas II, IIa, IIb, IIc, IId, and IIe, there is another embodiment comprising a compound in which R³ is C₁₋₁₀ alkyl, C₁₋₁₀ alkoxy, or C₁₋₁₀ heteroalkyl, each of which may be optionally substituted with from 1 to 5 R²⁰ groups, or a pharmaceutically acceptable salt thereof. In separate embodiments within each of the compounds described for Formulas II, IIa, IIb, IIc, IId, and IIe, there is another embodiment comprising a compound in which R³ is an, C₅₋₁₀ aryl, C₆₋₂₀ arylalkyl, C₅₋₁₀ heteroaryl, or C₆₋₂₀ heteroarylalkyl, each of which may be optionally substituted with from 1 to 5 R²⁰ groups, or a pharmaceutically acceptable salt thereof. In separate embodiments within each of the compounds described for Formulas II, IIa, IIb, IIc, and IId, there is another embodiment comprising a compound in which R⁵ is C₁₋₁₀ alkyl, or a pharmaceutically acceptable salt thereof. A separate embodiment comprises a compound of Formula IIe, as defined above, wherein R³ is C₁₋₁₀ alkyl, C₁₋₁₀ alkoxy, or C₁₋₁₀ heteroalkyl, each of which may be optionally substituted with from 1 to 5 R²⁰ groups, or a pharmaceutically acceptable salt thereof. There is also provided a separate embodiment with each of the embodiments described herein comprising a compound of Formula IIe, further in which R³ is C₅₋₁₀ aryl, C₆₋₂₀ arylalkyl, C₅₋₁₀ heteroaryl, or C₆₋₂₀ heteroarylalkyl, each of which may be optionally substituted with from 1 to 5 R²⁰ groups, or a pharmaceutically acceptable salt thereof.

In some embodiments, the modulator of a bromodomain-containing protein is a compound selected from the group below, or a pharmaceutically acceptable salt or hydrate thereof:

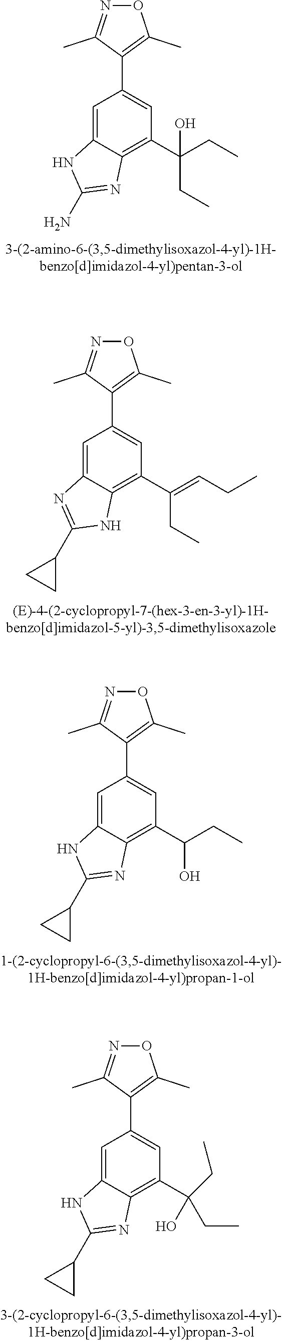 [SMILES: CCC(O)(CC)c1cc(-c2c(C)noc2C)cc2[nH]c(N)nc12 ; CC/C=C(\CC)c1cc(-c2c(C)noc2C)cc2nc(C3CC3)[nH]c12 ; CCC(O)c1cc(-c2c(C)noc2C)cc2[nH]c(C3CC3)nc12 ; CCC(O)(CC)c1cc(-c2c(C)noc2C)cc2[nH]c(C3CC3)nc12]

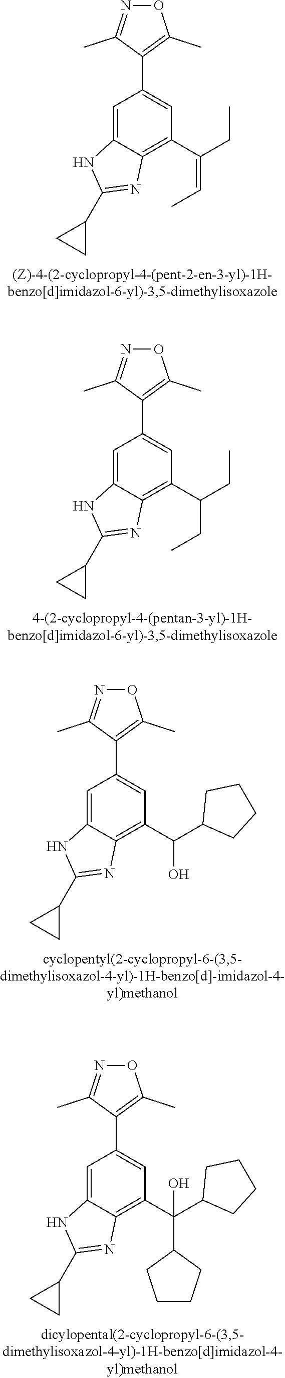 [SMILES: C/C=C(/CC)c1cc(-c2c(C)noc2C)cc2[nH]c(C3CC3)nc12 ; CCC(CC)c1cc(-c2c(C)noc2C)cc2[nH]c(C3CC3)nc12 ; Cc1noc(C)c1-c1cc(C(O)C2CCCC2)c2nc(C3CC3)[nH]c2c1 ; Cc1noc(C)c1-c1cc(C(O)(C2CCCC2)C2CCCC2)c2nc(C3CC3)[nH]c2c1]

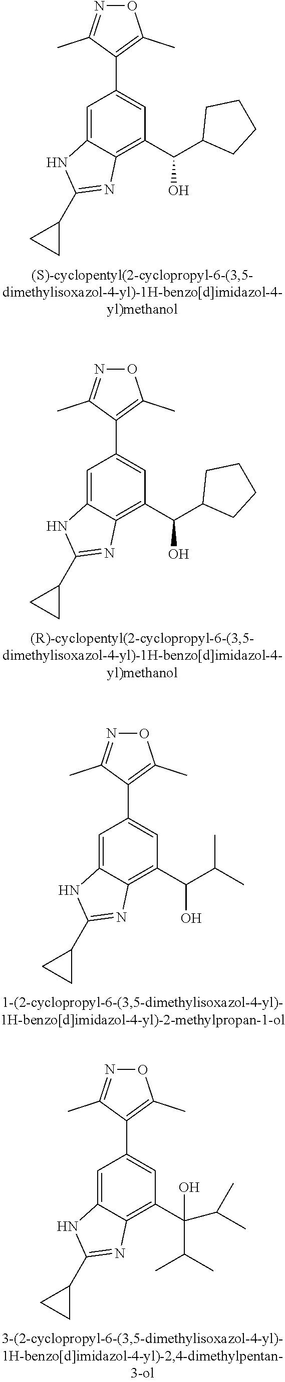 [SMILES: Cc1noc(C)c1-c1cc([C@@H](O)C2CCCC2)c2nc(C3CC3)[nH]c2c1 ; Cc1noc(C)c1-c1cc([C@H](O)C2CCCC2)c2nc(C3CC3)[nH]c2c1 ; Cc1noc(C)c1-c1cc(C(O)C(C)C)c2nc(C3CC3)[nH]c2c1 ; Cc1noc(C)c1-c1cc(C(O)(C(C)C)C(C)C)c2nc(C3CC3)[nH]c2c1]

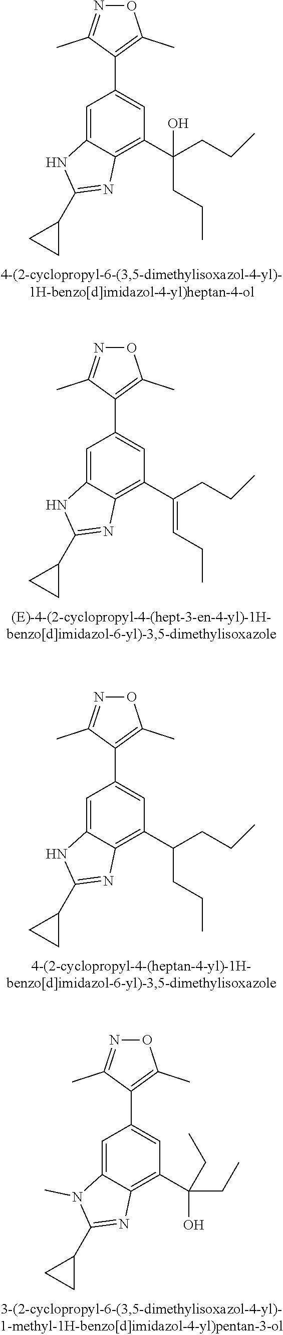

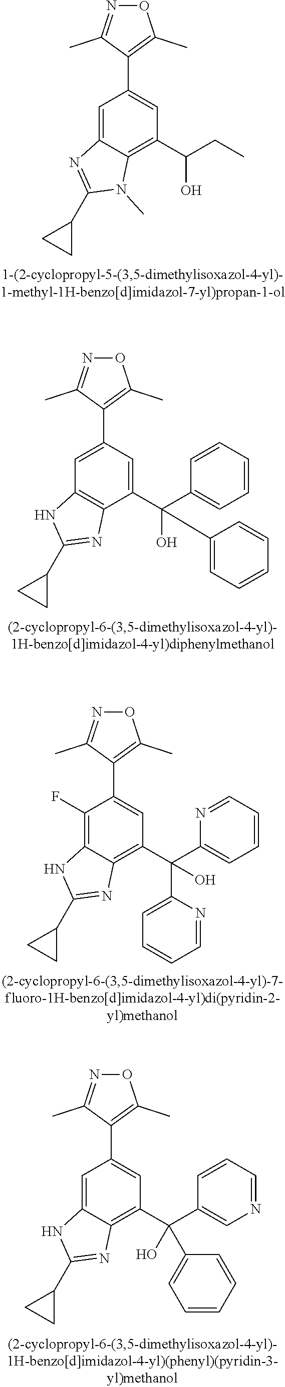 [SMILES: CCC(O)c1cc(-c2c(C)noc2C)cc2nc(C3CC3)n(C)c12 ; Cc1noc(C)c1-c1cc(C(O)(c2ccccc2)c2ccccc2)c2nc(C3CC3)[nH]c2c1 ; Cc1noc(C)c1-c1cc(C(O)(c2ccccn2)c2ccccn2)c2nc(C3CC3)[nH]c2c1F ; Cc1noc(C)c1-c1cc(C(O)(c2ccccc2)c2cccnc2)c2nc(C3CC3)[nH]c2c1]

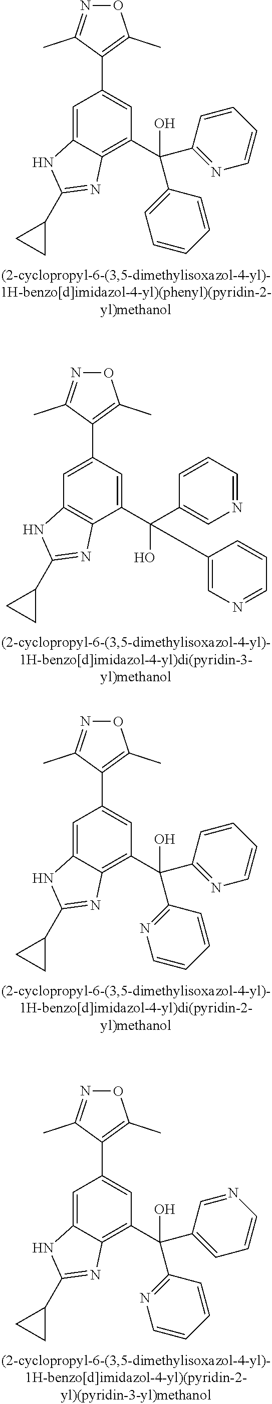 [SMILES: Cc1noc(C)c1-c1cc(C(O)(c2ccccc2)c2ccccn2)c2nc(C3CC3)[nH]c2c1 ; Cc1noc(C)c1-c1cc(C(O)(c2cccnc2)c2cccnc2)c2nc(C3CC3)[nH]c2c1 ; Cc1noc(C)c1-c1cc(C(O)(c2ccccn2)c2ccccn2)c2nc(C3CC3)[nH]c2c1 ; Cc1noc(C)c1-c1cc(C(O)(c2cccnc2)c2ccccn2)c2nc(C3CC3)[nH]c2c1]

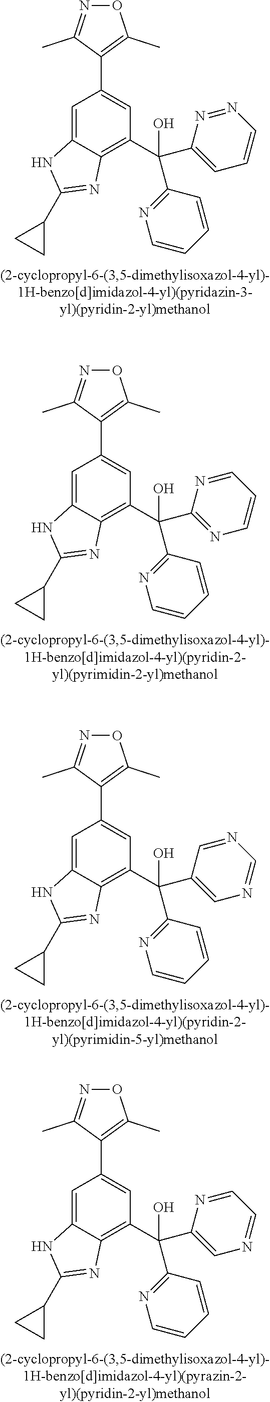 [SMILES: Cc1noc(C)c1-c1cc(C(O)(c2ccccn2)c2cccnn2)c2nc(C3CC3)[nH]c2c1 ; Cc1noc(C)c1-c1cc(C(O)(c2ccccn2)c2ncccn2)c2nc(C3CC3)[nH]c2c1 ; Cc1noc(C)c1-c1cc(C(O)(c2cncnc2)c2ccccn2)c2nc(C3CC3)[nH]c2c1 ; Cc1noc(C)c1-c1cc(C(O)(c2ccccn2)c2cnccn2)c2nc(C3CC3)[nH]c2c1]

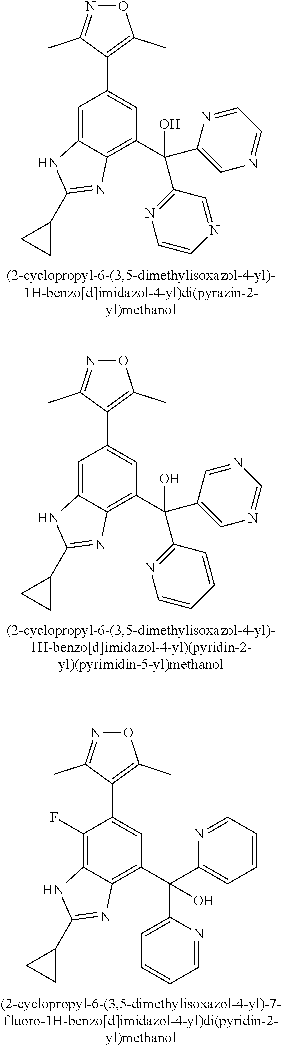 [SMILES: Cc1noc(C)c1-c1cc(C(O)(c2cnccn2)c2cnccn2)c2nc(C3CC3)[nH]c2c1 ; Cc1noc(C)c1-c1cc(C(O)(c2cncnc2)c2ccccn2)c2nc(C3CC3)[nH]c2c1 ; Cc1noc(C)c1-c1cc(C(O)(c2ccccn2)c2ccccn2)c2nc(C3CC3)[nH]c2c1F]

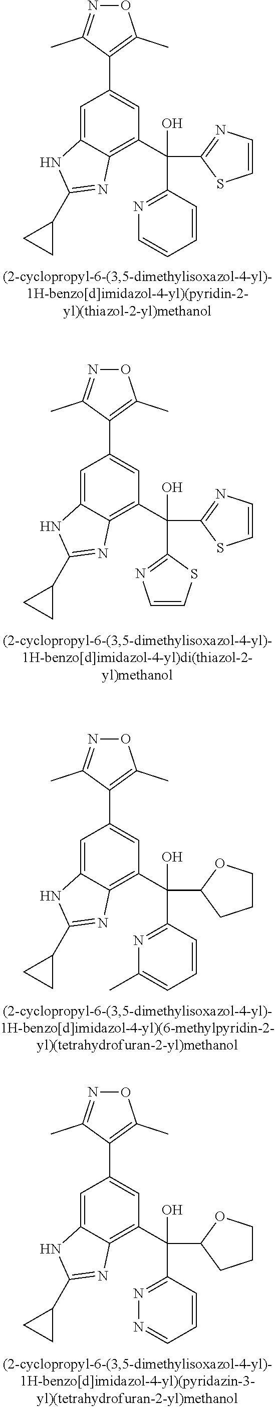 [SMILES: Cc1noc(C)c1-c1cc(C(O)(c2ccccn2)c2nccs2)c2nc(C3CC3)[nH]c2c1 ; Cc1noc(C)c1-c1cc(C(O)(c2nccs2)c2nccs2)c2nc(C3CC3)[nH]c2c1 ; Cc1cccc(C(O)(c2cc(-c3c(C)noc3C)cc3[nH]c(C4CC4)nc23)C2CCCO2)n1 ; Cc1noc(C)c1-c1cc(C(O)(c2cccnn2)C2CCCO2)c2nc(C3CC3)[nH]c2c1]

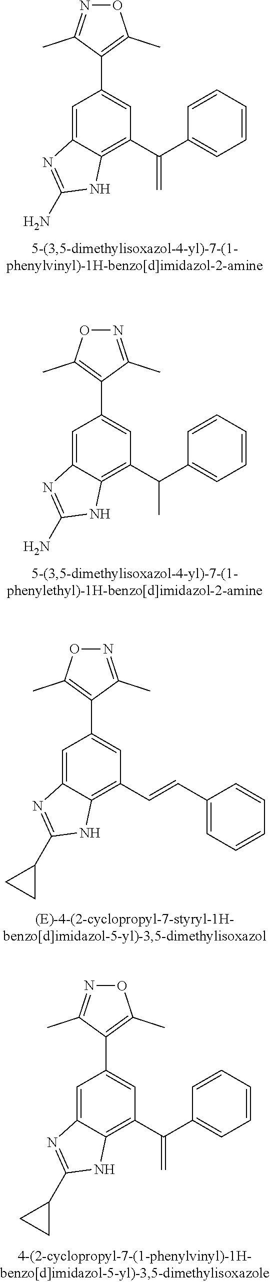 [SMILES: C=C(c1ccccc1)c1cc(-c2c(C)noc2C)cc2nc(N)[nH]c12 ; Cc1noc(C)c1-c1cc(C(C)c2ccccc2)c2[nH]c(N)nc2c1 ; Cc1noc(C)c1-c1cc(/C=C/c2ccccc2)c2[nH]c(C3CC3)nc2c1 ; C=C(c1ccccc1)c1cc(-c2c(C)noc2C)cc2nc(C3CC3)[nH]c12]

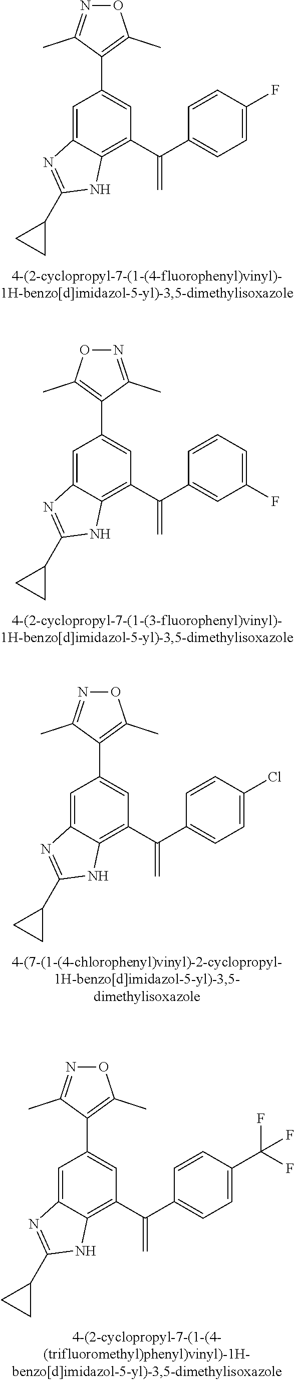 [SMILES: C=C(c1ccc(F)cc1)c1cc(-c2c(C)noc2C)cc2nc(C3CC3)[nH]c12 ; C=C(c1cccc(F)c1)c1cc(-c2c(C)noc2C)cc2nc(C3CC3)[nH]c12 ; C=C(c1ccc(Cl)cc1)c1cc(-c2c(C)noc2C)cc2nc(C3CC3)[nH]c12 ; C=C(c1ccc(C(F)(F)F)cc1)c1cc(-c2c(C)noc2C)cc2nc(C3CC3)[nH]c12]

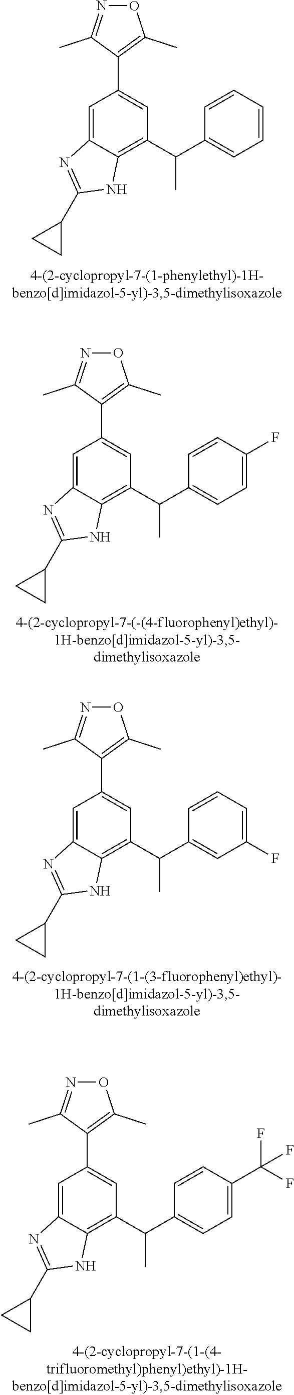 [SMILES: Cc1noc(C)c1-c1cc(C(C)c2ccccc2)c2[nH]c(C3CC3)nc2c1 ; Cc1noc(C)c1-c1cc(C(C)c2ccc(F)cc2)c2[nH]c(C3CC3)nc2c1 ; Cc1noc(C)c1-c1cc(C(C)c2cccc(F)c2)c2[nH]c(C3CC3)nc2c1 ; Cc1noc(C)c1-c1cc(C(C)c2ccc(C(F)(F)F)cc2)c2[nH]c(C3CC3)nc2c1]

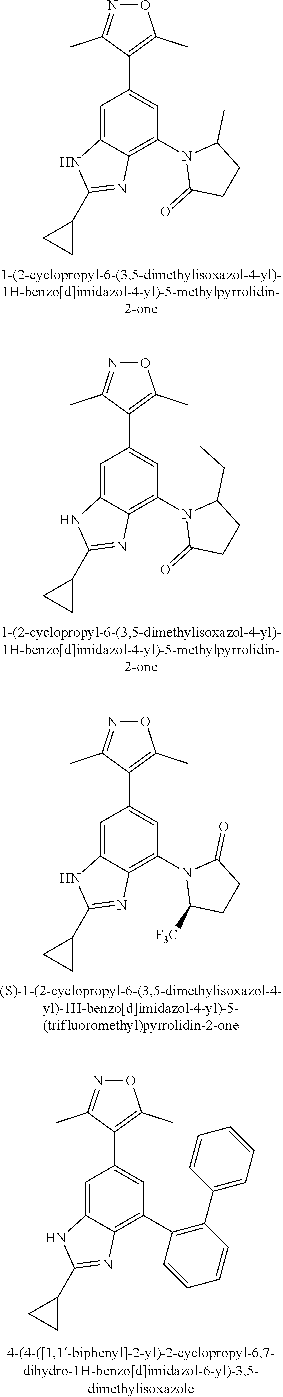 [SMILES: Cc1noc(C)c1-c1cc(N2C(=O)CCC2C)c2nc(C3CC3)[nH]c2c1 ; CCC1CCC(=O)N1c1cc(-c2c(C)noc2C)cc2[nH]c(C3CC3)nc12 ; Cc1noc(C)c1-c1cc(N2C(=O)CC[C@H]2C(F)(F)F)c2nc(C3CC3)[nH]c2c1 ; Cc1noc(C)c1-c1cc(-c2ccccc2-c2ccccc2)c2nc(C3CC3)[nH]c2c1]

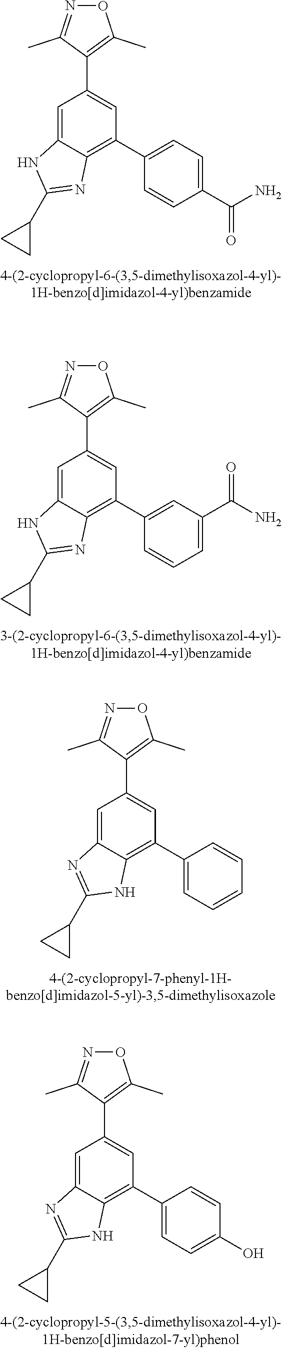 [SMILES: Cc1noc(C)c1-c1cc(-c2ccc(C(N)=O)cc2)c2nc(C3CC3)[nH]c2c1 ; Cc1noc(C)c1-c1cc(-c2cccc(C(N)=O)c2)c2nc(C3CC3)[nH]c2c1 ; Cc1noc(C)c1-c1cc(-c2ccccc2)c2[nH]c(C3CC3)nc2c1 ; Cc1noc(C)c1-c1cc(-c2ccc(O)cc2)c2[nH]c(C3CC3)nc2c1]

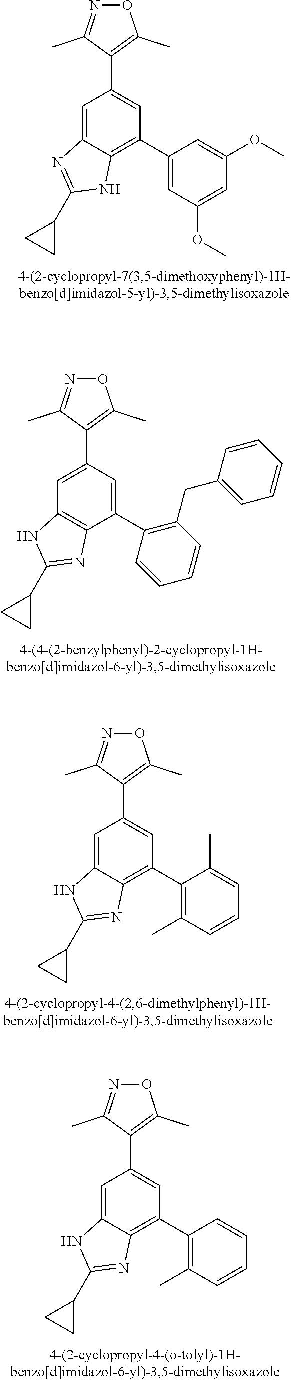 [SMILES: COc1cc(OC)cc(-c2cc(-c3c(C)noc3C)cc3nc(C4CC4)[nH]c23)c1 ; Cc1noc(C)c1-c1cc(-c2ccccc2Cc2ccccc2)c2nc(C3CC3)[nH]c2c1 ; Cc1cccc(C)c1-c1cc(-c2c(C)noc2C)cc2[nH]c(C3CC3)nc12 ; Cc1ccccc1-c1cc(-c2c(C)noc2C)cc2[nH]c(C3CC3)nc12]

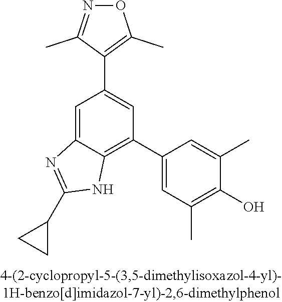 [SMILES: Cc1cc(-c2cc(-c3c(C)noc3C)cc3nc(C4CC4)[nH]c23)cc(C)c1O]

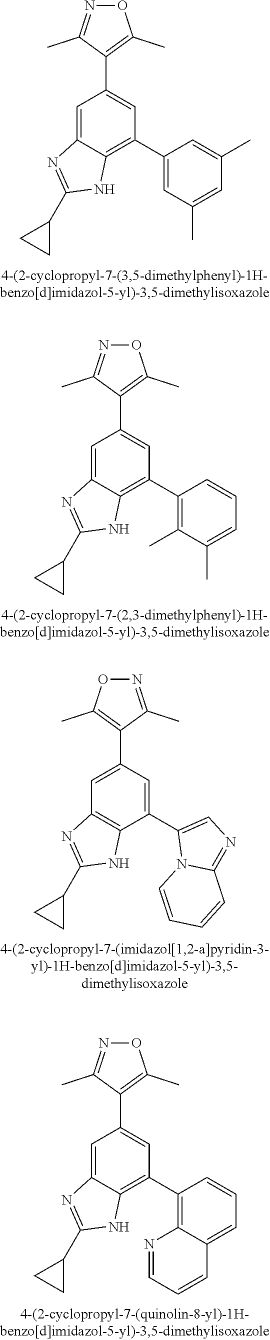 [SMILES: Cc1cc(C)cc(-c2cc(-c3c(C)noc3C)cc3nc(C4CC4)[nH]c23)c1 ; Cc1cccc(-c2cc(-c3c(C)noc3C)cc3nc(C4CC4)[nH]c23)c1C ; Cc1noc(C)c1-c1cc(-c2cnc3ccccn23)c2[nH]c(C3CC3)nc2c1 ; Cc1noc(C)c1-c1cc(-c2cccc3cccnc23)c2[nH]c(C3CC3)nc2c1]

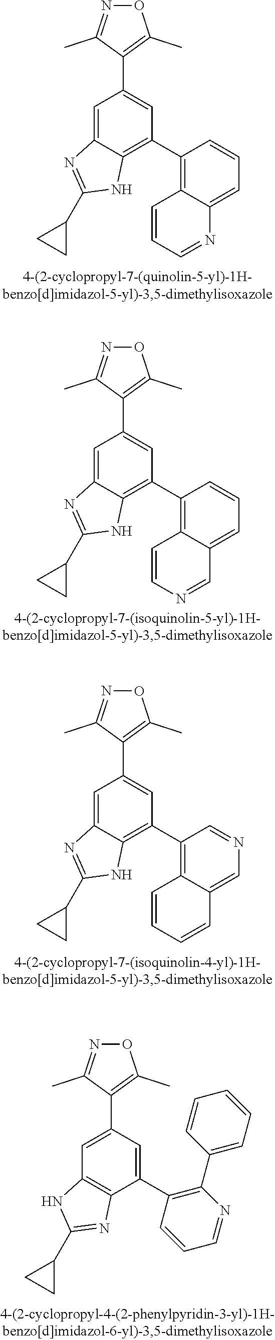 [SMILES: Cc1noc(C)c1-c1cc(-c2cccc3ncccc23)c2[nH]c(C3CC3)nc2c1 ; Cc1noc(C)c1-c1cc(-c2cccc3cnccc23)c2[nH]c(C3CC3)nc2c1 ; Cc1noc(C)c1-c1cc(-c2cncc3ccccc23)c2[nH]c(C3CC3)nc2c1 ; Cc1noc(C)c1-c1cc(-c2cccnc2-c2ccccc2)c2nc(C3CC3)[nH]c2c1]

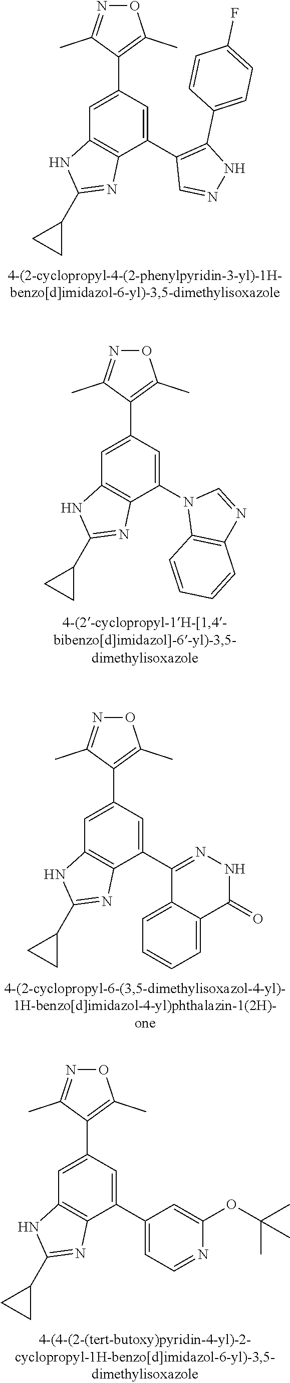 [SMILES: Cc1noc(C)c1-c1cc(-c2cn[nH]c2-c2ccc(F)cc2)c2nc(C3CC3)[nH]c2c1 ; Cc1noc(C)c1-c1cc(-n2cnc3ccccc32)c2nc(C3CC3)[nH]c2c1 ; Cc1noc(C)c1-c1cc(-c2n[nH]c(=O)c3ccccc23)c2nc(C3CC3)[nH]c2c1 ; Cc1noc(C)c1-c1cc(-c2ccnc(OC(C)(C)C)c2)c2nc(C3CC3)[nH]c2c1]

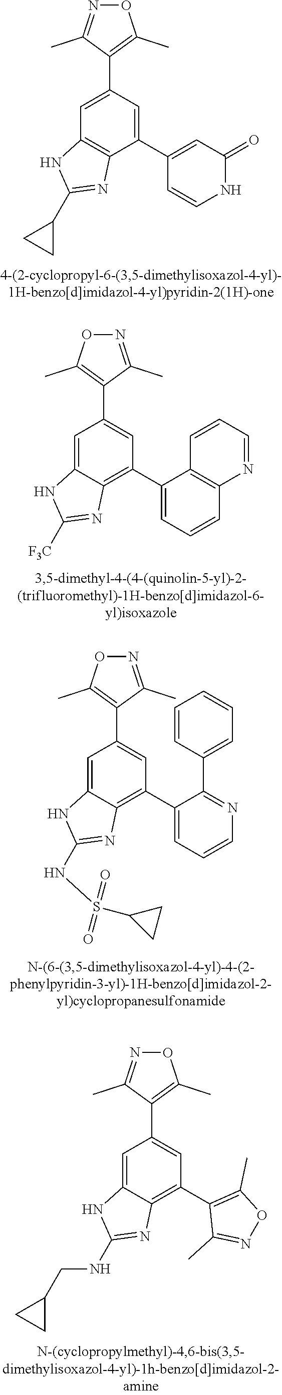 [SMILES: Cc1noc(C)c1-c1cc(-c2cc[nH]c(=O)c2)c2nc(C3CC3)[nH]c2c1 ; Cc1noc(C)c1-c1cc(-c2cccc3ncccc23)c2nc(C(F)(F)F)[nH]c2c1 ; Cc1noc(C)c1-c1cc(-c2cccnc2-c2ccccc2)c2nc(NS(=O)(=O)C3CC3)[nH]c2c1 ; Cc1noc(C)c1-c1cc(-c2c(C)noc2C)c2nc(NCC3CC3)[nH]c2c1]

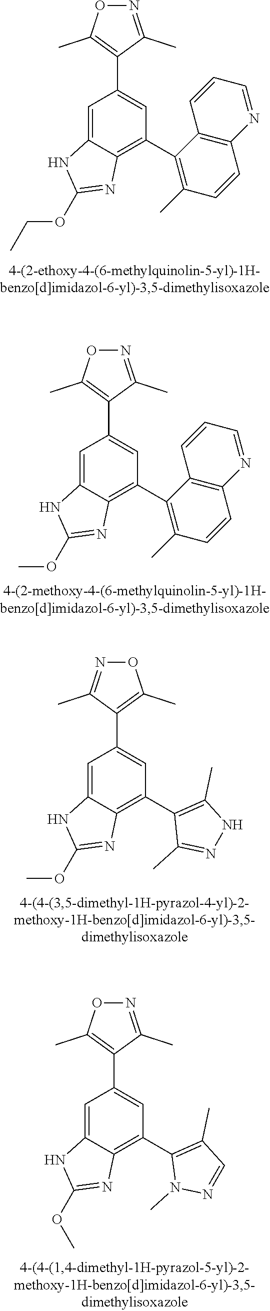 [SMILES: CCOc1nc2c(-c3c(C)ccc4ncccc34)cc(-c3c(C)noc3C)cc2[nH]1 ; COc1nc2c(-c3c(C)ccc4ncccc34)cc(-c3c(C)noc3C)cc2[nH]1 ; COc1nc2c(-c3c(C)n[nH]c3C)cc(-c3c(C)noc3C)cc2[nH]1 ; COc1nc2c(-c3c(C)cnn3C)cc(-c3c(C)noc3C)cc2[nH]1]

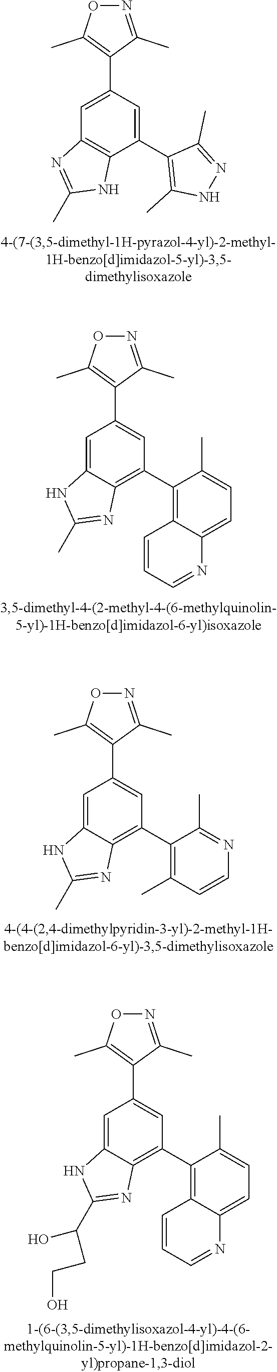 [SMILES: Cc1nc2cc(-c3c(C)noc3C)cc(-c3c(C)n[nH]c3C)c2[nH]1 ; Cc1nc2c(-c3c(C)ccc4ncccc34)cc(-c3c(C)noc3C)cc2[nH]1 ; Cc1nc2c(-c3c(C)ccnc3C)cc(-c3c(C)noc3C)cc2[nH]1 ; Cc1ccc2ncccc2c1-c1cc(-c2c(C)noc2C)cc2[nH]c(C(O)CCO)nc12]

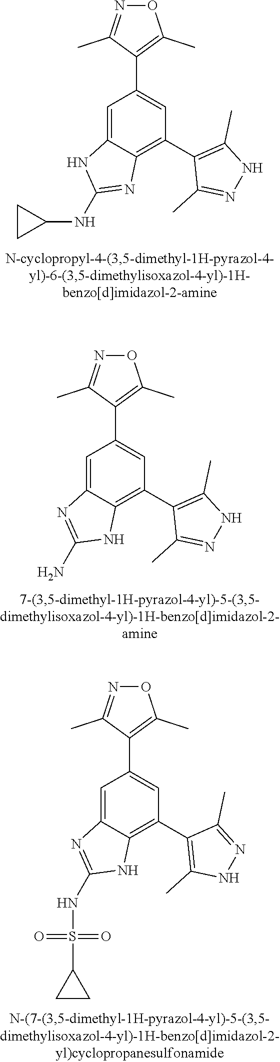 [SMILES: Cc1n[nH]c(C)c1-c1cc(-c2c(C)noc2C)cc2[nH]c(NC3CC3)nc12 ; Cc1n[nH]c(C)c1-c1cc(-c2c(C)noc2C)cc2nc(N)[nH]c12 ; Cc1n[nH]c(C)c1-c1cc(-c2c(C)noc2C)cc2nc(NS(=O)(=O)C3CC3)[nH]c12]

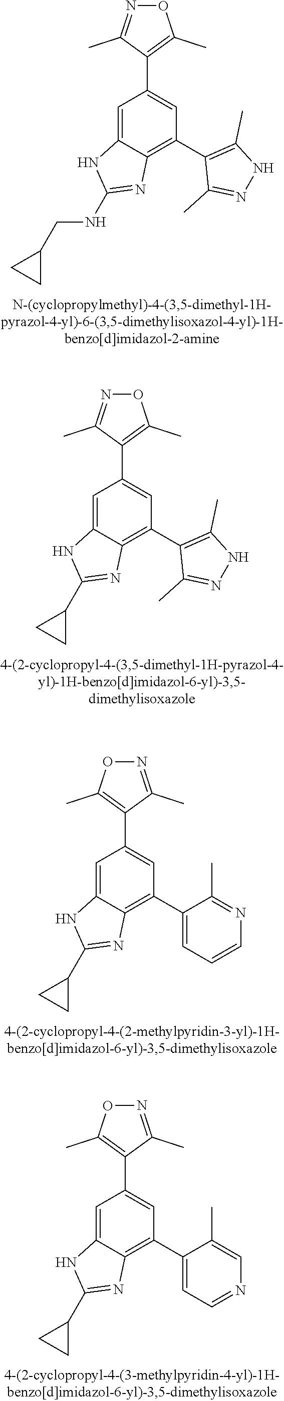 [SMILES: Cc1n[nH]c(C)c1-c1cc(-c2c(C)noc2C)cc2[nH]c(NCC3CC3)nc12 ; Cc1n[nH]c(C)c1-c1cc(-c2c(C)noc2C)cc2[nH]c(C3CC3)nc12 ; Cc1ncccc1-c1cc(-c2c(C)noc2C)cc2[nH]c(C3CC3)nc12 ; Cc1cnccc1-c1cc(-c2c(C)noc2C)cc2[nH]c(C3CC3)nc12]

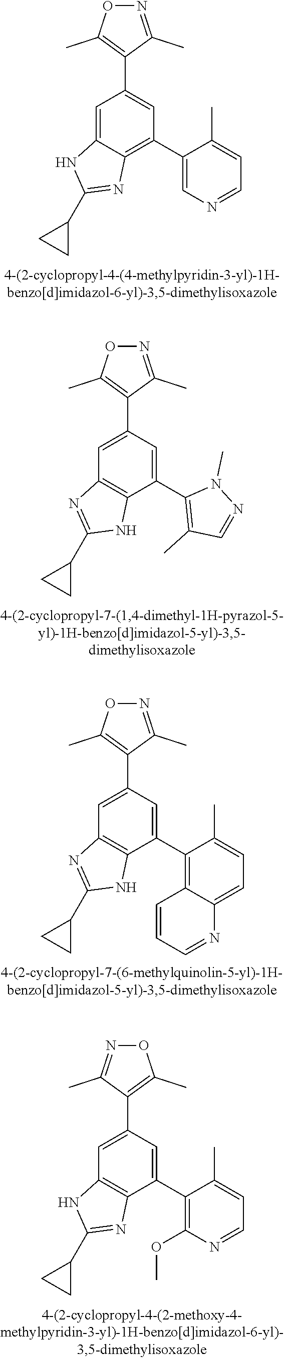 [SMILES: Cc1ccncc1-c1cc(-c2c(C)noc2C)cc2[nH]c(C3CC3)nc12 ; Cc1cnn(C)c1-c1cc(-c2c(C)noc2C)cc2nc(C3CC3)[nH]c12 ; Cc1ccc2ncccc2c1-c1cc(-c2c(C)noc2C)cc2nc(C3CC3)[nH]c12 ; COc1nccc(C)c1-c1cc(-c2c(C)noc2C)cc2[nH]c(C3CC3)nc12]

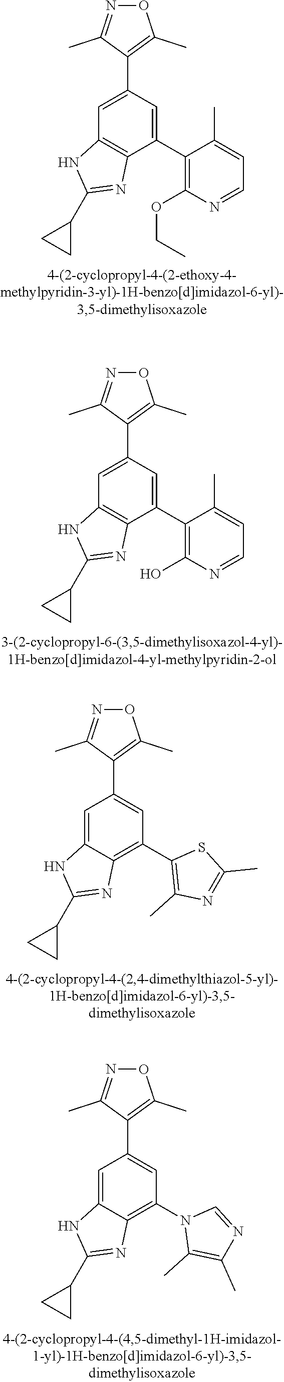 [SMILES: CCOc1nccc(C)c1-c1cc(-c2c(C)noc2C)cc2[nH]c(C3CC3)nc12 ; Cc1ccnc(O)c1-c1cc(-c2c(C)noc2C)cc2[nH]c(C3CC3)nc12 ; Cc1nc(C)c(-c2cc(-c3c(C)noc3C)cc3[nH]c(C4CC4)nc23)s1 ; Cc1noc(C)c1-c1cc(-n2cnc(C)c2C)c2nc(C3CC3)[nH]c2c1]

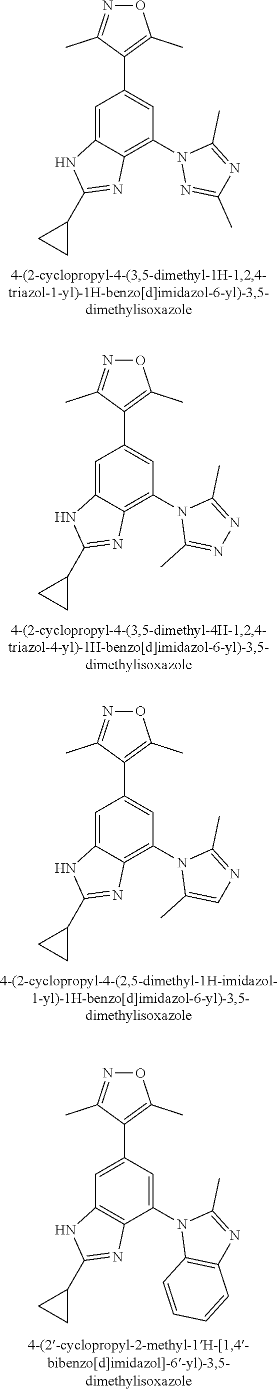 [SMILES: Cc1nc(C)n(-c2cc(-c3c(C)noc3C)cc3[nH]c(C4CC4)nc23)n1 ; Cc1noc(C)c1-c1cc(-n2c(C)nnc2C)c2nc(C3CC3)[nH]c2c1 ; Cc1noc(C)c1-c1cc(-n2c(C)cnc2C)c2nc(C3CC3)[nH]c2c1 ; Cc1noc(C)c1-c1cc(-n2c(C)nc3ccccc32)c2nc(C3CC3)[nH]c2c1]

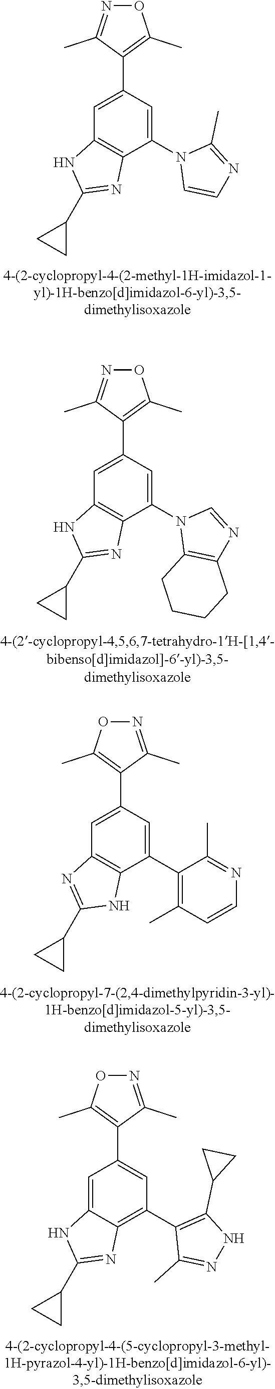 [SMILES: Cc1noc(C)c1-c1cc(-n2ccnc2C)c2nc(C3CC3)[nH]c2c1 ; Cc1noc(C)c1-c1cc(-n2cnc3c2CCCC3)c2nc(C3CC3)[nH]c2c1 ; Cc1ccnc(C)c1-c1cc(-c2c(C)noc2C)cc2nc(C3CC3)[nH]c12 ; Cc1n[nH]c(C2CC2)c1-c1cc(-c2c(C)noc2C)cc2[nH]c(C3CC3)nc12]

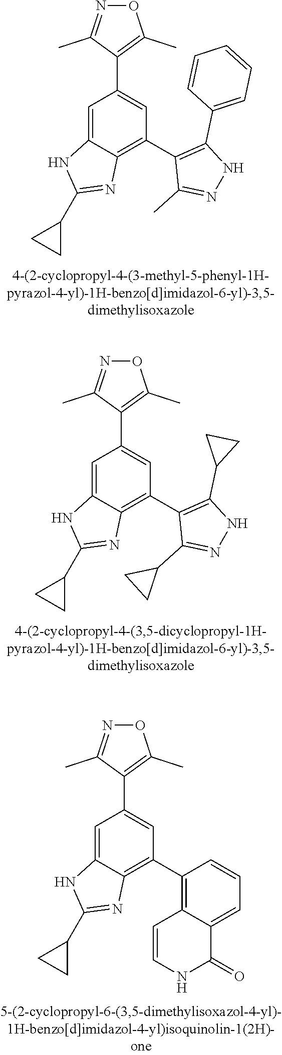 [SMILES: Cc1n[nH]c(-c2ccccc2)c1-c1cc(-c2c(C)noc2C)cc2[nH]c(C3CC3)nc12 ; Cc1noc(C)c1-c1cc(-c2c(C3CC3)n[nH]c2C2CC2)c2nc(C3CC3)[nH]c2c1 ; Cc1noc(C)c1-c1cc(-c2cccc3c(=O)[nH]ccc23)c2nc(C3CC3)[nH]c2c1]

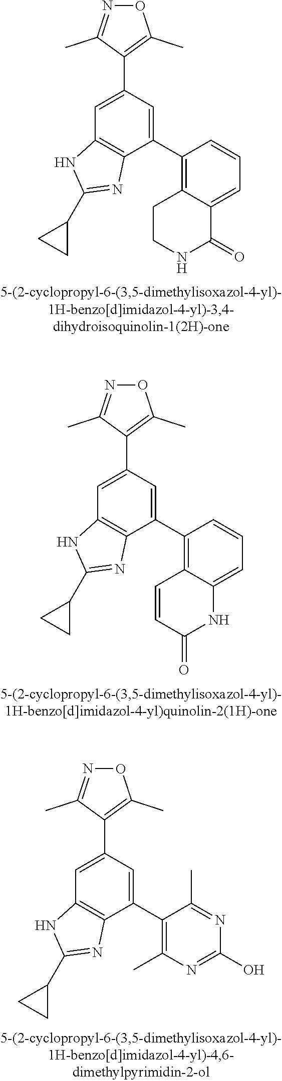 [SMILES: Cc1noc(C)c1-c1cc(-c2cccc3c2CCNC3=O)c2nc(C3CC3)[nH]c2c1 ; Cc1noc(C)c1-c1cc(-c2cccc3[nH]c(=O)ccc23)c2nc(C3CC3)[nH]c2c1 ; Cc1nc(O)nc(C)c1-c1cc(-c2c(C)noc2C)cc2[nH]c(C3CC3)nc12]

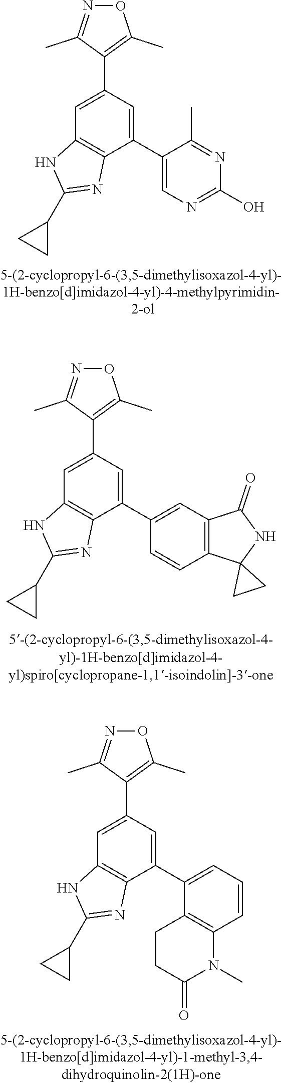 [SMILES: Cc1nc(O)ncc1-c1cc(-c2c(C)noc2C)cc2[nH]c(C3CC3)nc12 ; Cc1noc(C)c1-c1cc(-c2ccc3c(c2)C(=O)NC32CC2)c2nc(C3CC3)[nH]c2c1 ; Cc1noc(C)c1-c1cc(-c2cccc3c2CCC(=O)N3C)c2nc(C3CC3)[nH]c2c1]

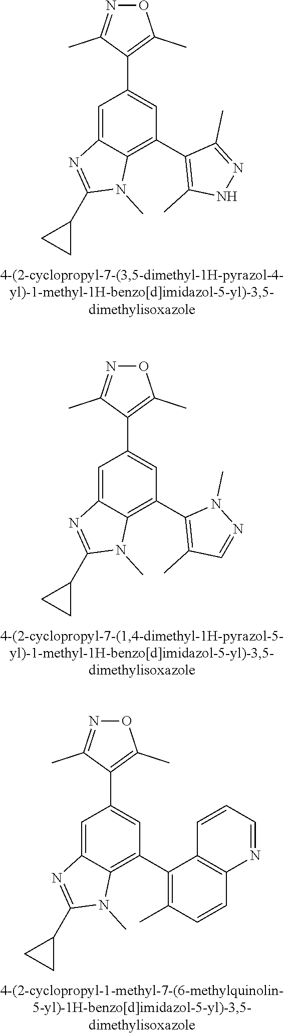 [SMILES: Cc1n[nH]c(C)c1-c1cc(-c2c(C)noc2C)cc2nc(C3CC3)n(C)c12 ; Cc1cnn(C)c1-c1cc(-c2c(C)noc2C)cc2nc(C3CC3)n(C)c12 ; Cc1ccc2ncccc2c1-c1cc(-c2c(C)noc2C)cc2nc(C3CC3)n(C)c12]

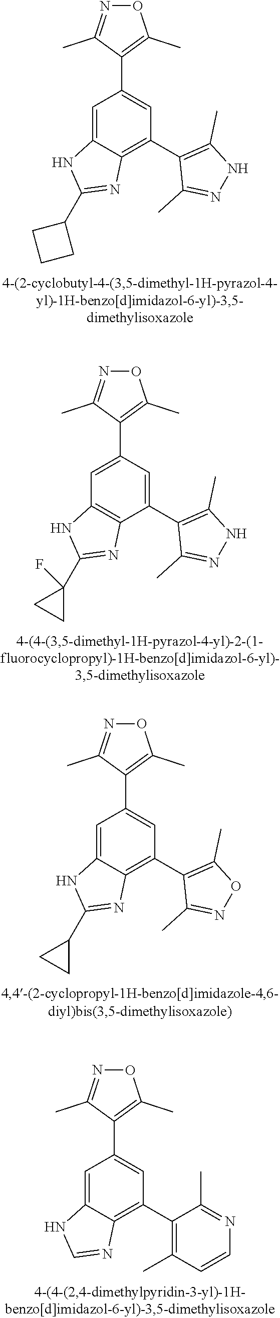 [SMILES: Cc1n[nH]c(C)c1-c1cc(-c2c(C)noc2C)cc2[nH]c(C3CCC3)nc12 ; Cc1n[nH]c(C)c1-c1cc(-c2c(C)noc2C)cc2[nH]c(C3(F)CC3)nc12 ; Cc1noc(C)c1-c1cc(-c2c(C)noc2C)c2nc(C3CC3)[nH]c2c1 ; Cc1ccnc(C)c1-c1cc(-c2c(C)noc2C)cc2[nH]cnc12]

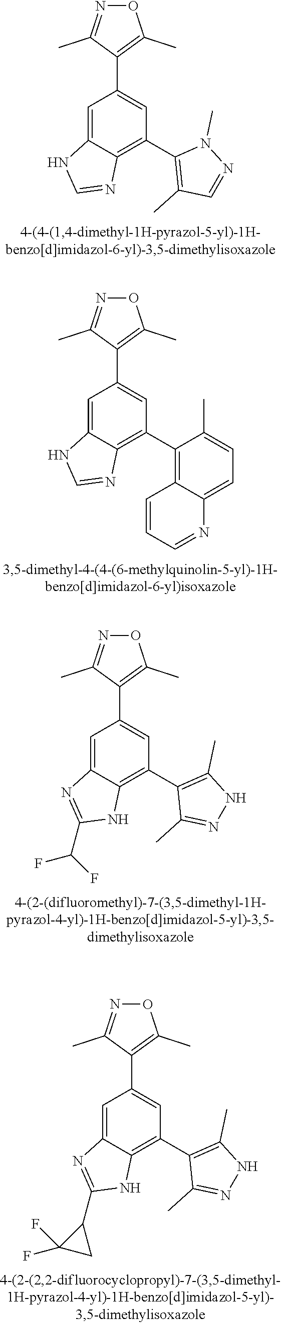 [SMILES: Cc1cnn(C)c1-c1cc(-c2c(C)noc2C)cc2[nH]cnc12 ; Cc1ccc2ncccc2c1-c1cc(-c2c(C)noc2C)cc2[nH]cnc12 ; Cc1n[nH]c(C)c1-c1cc(-c2c(C)noc2C)cc2nc(C(F)F)[nH]c12 ; Cc1n[nH]c(C)c1-c1cc(-c2c(C)noc2C)cc2nc(C3CC3(F)F)[nH]c12]

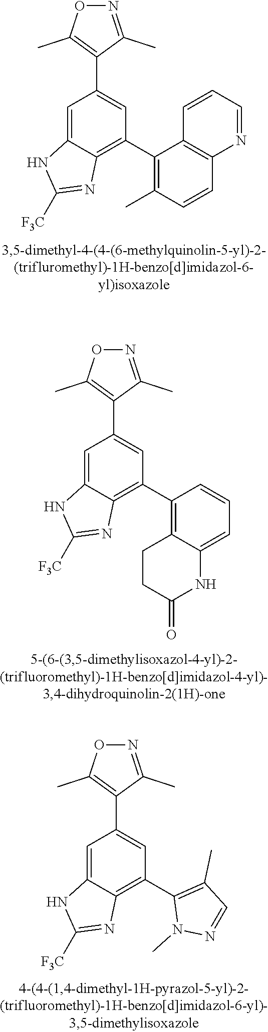 [SMILES: Cc1ccc2ncccc2c1-c1cc(-c2c(C)noc2C)cc2[nH]c(C(F)(F)F)nc12 ; Cc1noc(C)c1-c1cc(-c2cccc3c2CCC(=O)N3)c2nc(C(F)(F)F)[nH]c2c1 ; Cc1cnn(C)c1-c1cc(-c2c(C)noc2C)cc2[nH]c(C(F)(F)F)nc12]

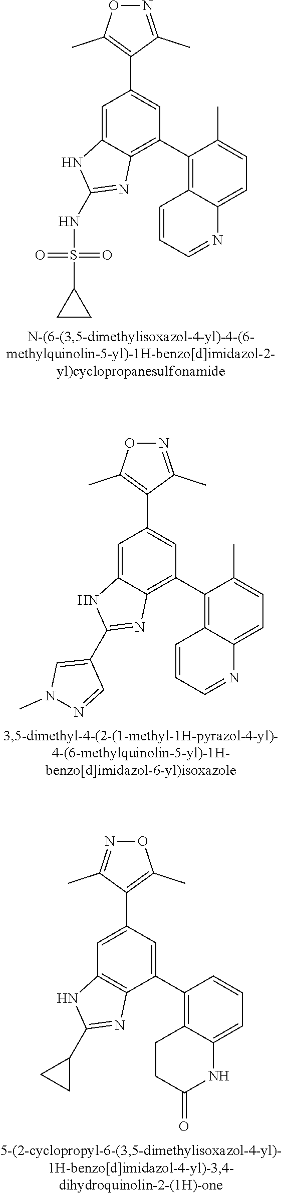 [SMILES: Cc1ccc2ncccc2c1-c1cc(-c2c(C)noc2C)cc2[nH]c(NS(=O)(=O)C3CC3)nc12 ; Cc1ccc2ncccc2c1-c1cc(-c2c(C)noc2C)cc2[nH]c(-c3cnn(C)c3)nc12 ; Cc1noc(C)c1-c1cc(-c2cccc3c2CCC(=O)N3)c2nc(C3CC3)[nH]c2c1]

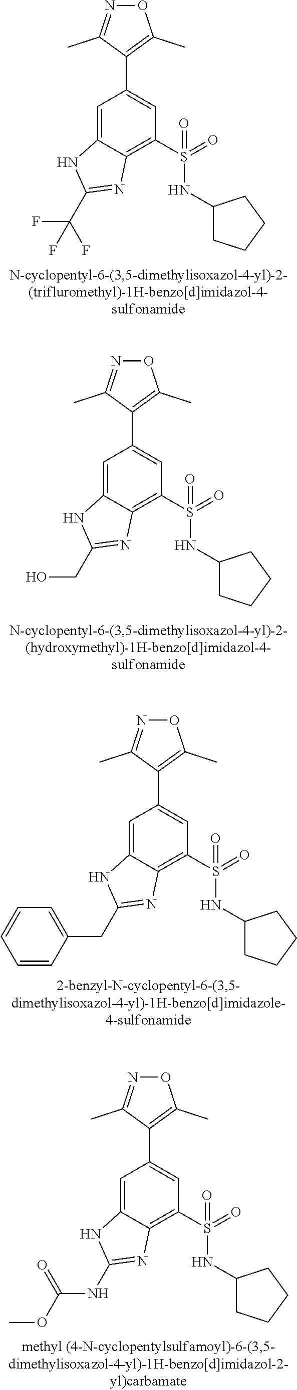 [SMILES: Cc1noc(C)c1-c1cc(S(=O)(=O)NC2CCCC2)c2nc(C(F)(F)F)[nH]c2c1 ; Cc1noc(C)c1-c1cc(S(=O)(=O)NC2CCCC2)c2nc(CO)[nH]c2c1 ; Cc1noc(C)c1-c1cc(S(=O)(=O)NC2CCCC2)c2nc(Cc3ccccc3)[nH]c2c1 ; COC(=O)Nc1nc2c(S(=O)(=O)NC3CCCC3)cc(-c3c(C)noc3C)cc2[nH]1]

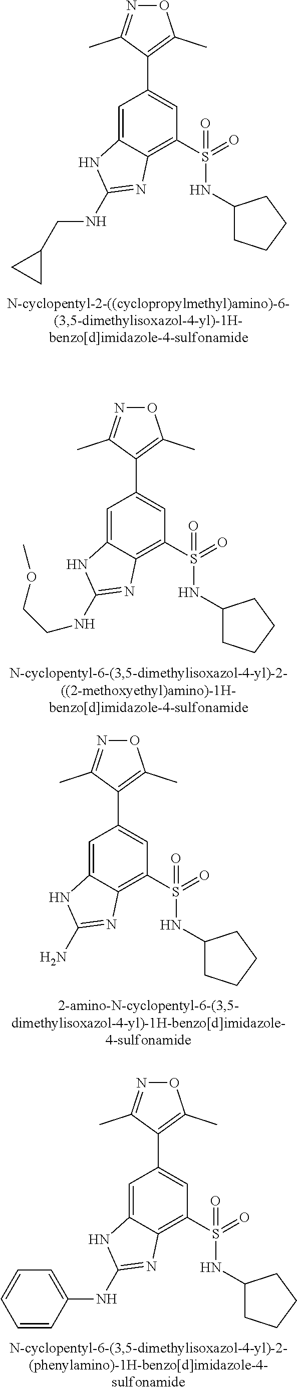 [SMILES: Cc1noc(C)c1-c1cc(S(=O)(=O)NC2CCCC2)c2nc(NCC3CC3)[nH]c2c1 ; COCCNc1nc2c(S(=O)(=O)NC3CCCC3)cc(-c3c(C)noc3C)cc2[nH]1 ; Cc1noc(C)c1-c1cc(S(=O)(=O)NC2CCCC2)c2nc(N)[nH]c2c1 ; Cc1noc(C)c1-c1cc(S(=O)(=O)NC2CCCC2)c2nc(Nc3ccccc3)[nH]c2c1]

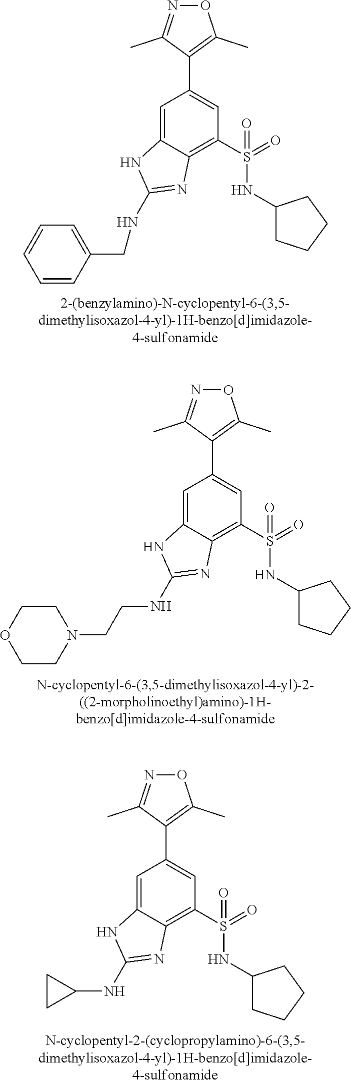 [SMILES: Cc1noc(C)c1-c1cc(S(=O)(=O)NC2CCCC2)c2nc(NCc3ccccc3)[nH]c2c1 ; Cc1noc(C)c1-c1cc(S(=O)(=O)NC2CCCC2)c2nc(NCCN3CCOCC3)[nH]c2c1 ; Cc1noc(C)c1-c1cc(S(=O)(=O)NC2CCCC2)c2nc(NC3CC3)[nH]c2c1]

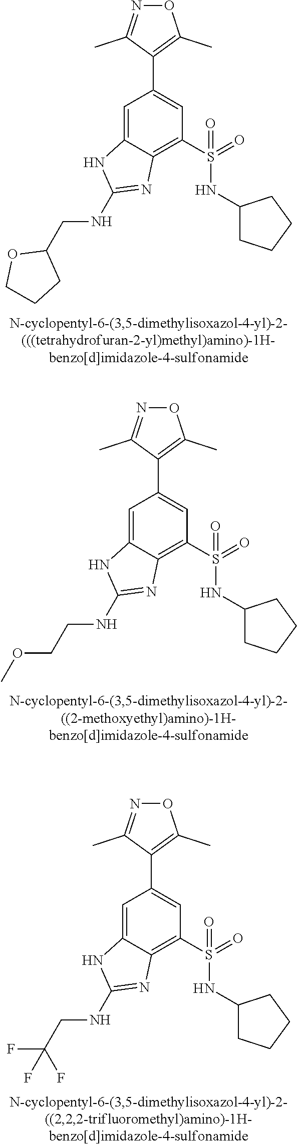 [SMILES: Cc1noc(C)c1-c1cc(S(=O)(=O)NC2CCCC2)c2nc(NCC3CCCO3)[nH]c2c1 ; COCCNc1nc2c(S(=O)(=O)NC3CCCC3)cc(-c3c(C)noc3C)cc2[nH]1 ; Cc1noc(C)c1-c1cc(S(=O)(=O)NC2CCCC2)c2nc(NCC(F)(F)F)[nH]c2c1]

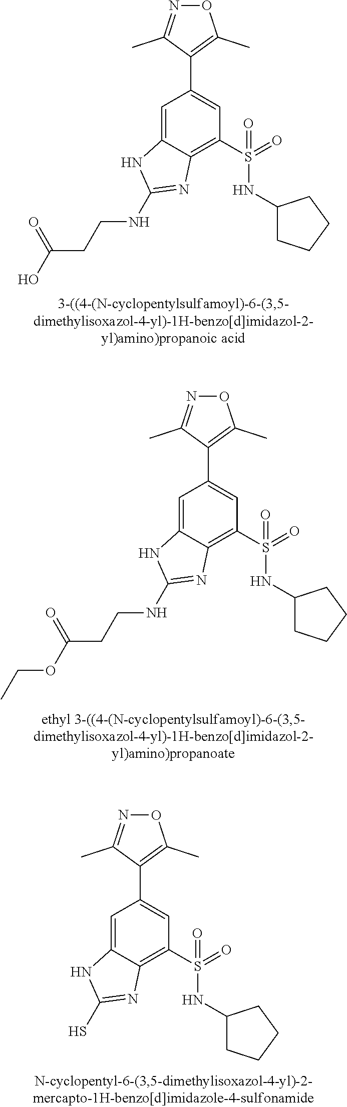 [SMILES: Cc1noc(C)c1-c1cc(S(=O)(=O)NC2CCCC2)c2nc(NCCC(=O)O)[nH]c2c1 ; CCOC(=O)CCNc1nc2c(S(=O)(=O)NC3CCCC3)cc(-c3c(C)noc3C)cc2[nH]1 ; Cc1noc(C)c1-c1cc(S(=O)(=O)NC2CCCC2)c2nc(S)[nH]c2c1]

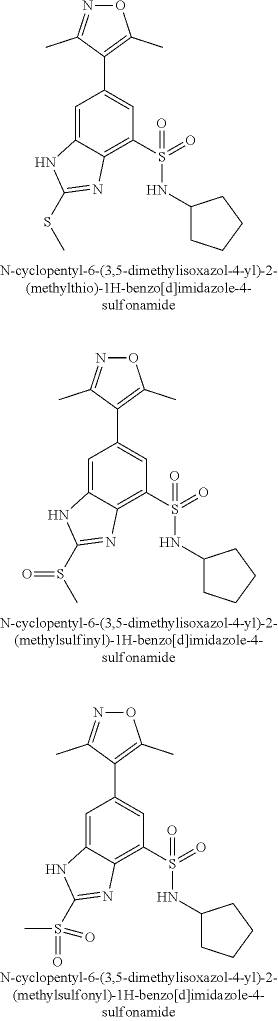 [SMILES: CSc1nc2c(S(=O)(=O)NC3CCCC3)cc(-c3c(C)noc3C)cc2[nH]1 ; Cc1noc(C)c1-c1cc(S(=O)(=O)NC2CCCC2)c2nc(S(C)=O)[nH]c2c1 ; Cc1noc(C)c1-c1cc(S(=O)(=O)NC2CCCC2)c2nc(S(C)(=O)=O)[nH]c2c1]

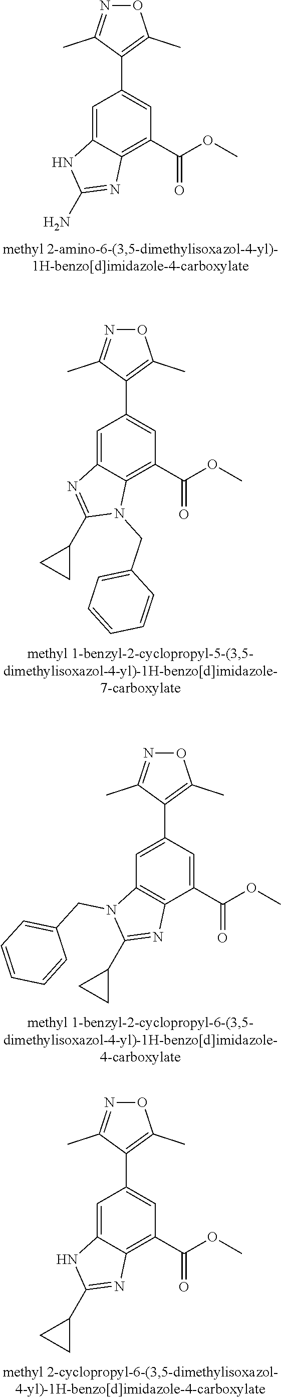

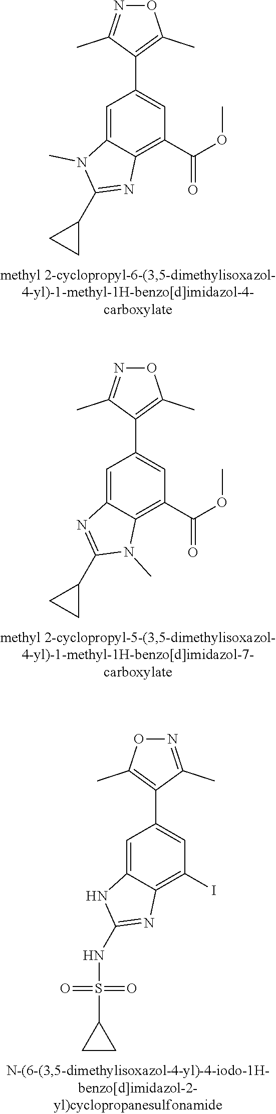

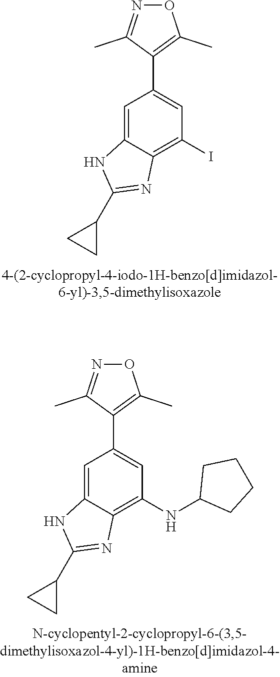 [SMILES: Cc1noc(C)c1-c1cc(I)c2nc(C3CC3)[nH]c2c1 ; Cc1noc(C)c1-c1cc(NC2CCCC2)c2nc(C3CC3)[nH]c2c1]

It is understood that separate, single embodiments comprise the methods, regimens, kits, and articles of manufacture in which the modulator of a bromodomain-containing protein is each separate compound listed in the table above. For instance, in one embodiment of each of the methods, regimens, kits, and articles of manufacture discussed herein, there is an embodiment in which the modulator of a bromodomain-containing protein is, (2-cyclopropyl-6-(3,5-dimethylisoxazol-4-yl)-l H-benzo[d]imidazol-4-yl)di(pyridin-2-yl)methanol, or a pharmaceutically acceptable hydrate thereof. In separate other embodiments for each of the methods, regimens, kits, and articles of manufacture discussed herein, there is an embodiment in which the modulator of a bromodomain-containing protein is (2-cyclopropyl-6-(3,5-dimethylisoxazol-4-yl)-H-benzo[d]imidazol-4-yl)di(pyrazin-2-yl)methanol, or a pharmaceutically acceptable hydrate thereof. There are also embodiments in which the modulator of a bromodomain-containing protein is (2-cyclopropyl-6-(3,5-dimethylisoxazol-4-yl)-1H-benzo[d]imidazol-4-yl)(pyridin-2-yl)(pyrimidin-5-yl)methanol, or a pharmaceutically acceptable hydrate thereof.

There are also embodiments in which the modulator of a bromodomain-containing protein is (2-cyclopropyl-6-(3,5-dimethylisoxazol-4-yl)-1H-benzo[d]imidazol-4-yl)(pyridin-2-yl)(pyrimidin-2-yl)methanol, or a pharmaceutically acceptable hydrate thereof. There are also embodiments in which the modulator of a bromodomain-containing protein is (2-cyclopropyl-6-(3,5-dimethylisoxazol-4-yl)-1H-benzo[d]imidazol-4-yl)di(pyridin-3-yl)methanol, or a pharmaceutically acceptable hydrate thereof. There are also embodiments in which the modulator of a bromodomain-containing protein is (2-cyclopropyl-6-(3,5-dimethylisoxazol-4-yl)-1H-benzo[d]imidazol-4-yl)(phenyl)(pyridin-2-yl)methanol, or a pharmaceutically acceptable hydrate thereof. There are also embodiments in which the modulator of a bromodomain-containing protein is (2-cyclopropyl-6-(3,5-dimethylisoxazol-4-yl)-1H-benzo[d]imidazol-4-yl)(phenylpyridin-3-yl)methanol, or a pharmaceutically acceptable hydrate thereof. The compounds which are modulators of a bromo-domain containing protein described above may be prepared as taught in US 2014-0336190.

### MMP9 Inhibiting Agents

Useful MMP9 inhibiting agents include comprises binding proteins, e.g., antibodies and antigen-binding fragments thereof, that bind to the matrix metalloproteinase-9 (MMP9) protein (MMP9 is also known as gelatinase-B), wherein the binding proteins comprise an immunoglobulin (Ig) heavy chain (or functional fragment thereof) and an Ig light chain (or functional fragment thereof) disclosed in U.S. 2015-0140580 (Smith et al.) and U.S. Pat. No. 8,377,443 (McAuley et al.), U.S. Pat. No. 8,501,916 (McAuley et al.), and U.S. Pat. No. 9,120,863 (McAuley et al.), each of which is incorporated herein by reference.

Practice of the present disclosure employs, unless otherwise indicated, standard methods and conventional techniques in the fields of cell biology, toxicology, molecular biology, biochemistry, cell culture, immunology, oncology, recombinant DNA and related fields as are within the skill of the art. Such techniques are described in the literature and thereby available to those of skill in the art. See, for example, Alberts, B. et al., “Molecular Biology of the Cell,” 5^{th} edition, Garland Science, New York, N.Y., 2008; Voet, D. et al. “Fundamentals of Biochemistry: Life at the Molecular Level,” 3^{rd} edition, John Wiley & Sons, Hoboken, N.J., 2008; Sambrook, J. et al., “Molecular Cloning: A Laboratory Manual,” 3^{rd} edition, Cold Spring Harbor Laboratory Press, 2001; Ausubel, F. et al., “Current Protocols in Molecular Biology,” John Wiley & Sons, New York, 1987 and periodic updates; Freshney, R. I., “Culture of Animal Cells: A Manual of Basic Technique,” 4^{th} edition, John Wiley & Sons, Somerset, N J, 2000; and the series “Methods in Enzymology,” Academic Press, San Diego, Calif. See also, for example, “Current Protocols in Immunology,” (R. Coico, series editor), Wiley, last updated August 2010.

The present combinations provide binding proteins, e.g., antibodies and antigen-binding fragments thereof, that bind to the matrix metalloproteinase-9 (MMP9) protein (MMP9 is also known as gelatinase-B). The binding proteins of the present disclosure generally comprise an immunoglobulin (Ig) heavy chain (or functional fragment thereof) and an Ig light chain (or functional fragment thereof) to be used in the methods, regimens, kits, and articles of manufacture herein with a pharmaceutically effective amount, or with individual dose units containing a pharmaceutically effective amount, of Compound A1.

The combinations include MMP9 binding proteins that bind specifically to MMP9 and not to other matrix metalloproteinases such as MMP1, MMP2, MMP3, MMP7, MMP9, MMP10, MMP12, MMP13. Such specific MMP9 binding proteins are thus generally not significantly or detectably crossreactive with non-MMP9 matrix metalloproteinases. MMP9 binding proteins that specifically bind MMP9 find use in applications in which it is necessary or desirable to obtain specific modulation (e.g., inhibition) of MMP9, e.g., without directly affecting the activity of other matrix metalloproteinases.

In certain embodiments of the present disclosure an anti-MMP9 antibody is an inhibitor of the activity of MMP9, and can be a specific inhibitor of MMP9. In particular, the MMP9 binding proteins disclosed herein will be useful for inhibition of MMP9 while allowing normal function of other, related matrix metalloproteinases. “An inhibitor of MMP” or “inhibitor of MMP9 activity” can be an antibody or an antigen binding fragment thereof that directly or indirectly inhibits activity of MMP9, including but not limited to enzymatic processing, inhibiting action of MMP9 on it substrate (e.g., by inhibiting substrate binding, substrate cleavage, and the like), and the like.

The present combinations also comprise MMP9 binding proteins that specifically bind to non-mouse MMP9, such as human MMP9, Cynomolgus monkey MMP9, and rat MMP9. The combinations also comprise MMP9 binding proteins (e.g., anti-MMP9 antibodies and functional fragments thereof) that act as non-competitive inhibitors. A “non-competitive inhibitor” refers to an inhibitor binds at site away from substrate binding site of an enzyme, and thus can bind the enzyme and effect inhibitory activity regardless of whether or not the enzyme is bound to its substrate, such non-competitive inhibitors can, for example, provide for a level of inhibition that can be substantially independent of substrate concentration.

MMP9 binding proteins (e.g., antibodies and functional fragments thereof) of the present disclosure include those that bind MMP9, particularly human MMP9, and having a heavy chain polypeptide (or functional fragment thereof) that has at least about 80%, 85%, 90%, 95% or more amino acid sequence identity to a heavy chain polypeptide disclosed herein. MMP9 binding proteins (e.g., antibodies and functional fragments thereof) of the present combinations, methods, articles of manufacture, and kits include those that bind MMP9, particularly human MMP9, and having a light polypeptide (or functional fragment thereof) that has at least about 80%, 85%, 90%, 95% or more amino acid sequence identity to a heavy chain polypeptide disclosed herein. MMP9 binding proteins (e.g., antibodies and functional fragments thereof) of the present disclosure include those that bind MMP9, particularly human MMP9, and have a heavy chain polypeptide (or functional fragment thereof) having the complementarity determining regions (“CDRs”) of heavy chain polypeptide and the CDRs of a light chain polypeptide (or functional fragment thereof) as disclosed herein.

MMP9 binding proteins including antibodies and functional fragments thereof. Accordingly, the present disclosure provides embodiments comprising, for example, antibodies or antigen binding fragments thereof, comprising a heavy chain variable region polypeptide having at least 80%, 85%, 90%, 95%, or greater amino acid sequence identity to an amino acid sequence of a heavy chain variable region described herein (e.g., SEQ ID NOS:1 or 5-8), and a variable light chain polypeptide having at least 80%, 85%, 90%, 95%, or greater amino acid sequence identity to an amino acid sequence of a light chain polypeptide as set forth herein (e.g., SEQ ID NOS:2 or 9-12).

Sequence identity between two nucleic acids can also be described in terms of hybridization of two molecules to each other under stringent conditions. The hybridization conditions are selected following standard methods in the art (see, for example, Sambrook, et al., Molecular Cloning: A Laboratory Manual, Second Edition, (1989) Cold Spring Harbor, N.Y.). An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH7.6), 5×Denhardt's solution, 10% dextran sulfate, and 20 mg/ml denatured, sheared salmon sperm DNA, followed by washing the filters in 0.1×SSC at about 65° C. Stringent hybridization conditions are hybridization conditions that are at least as stringent as the above representative conditions, where conditions are considered to be at least as stringent if they are at least about 80% as stringent, typically at least 90% as stringent as the above specific stringent conditions. Examples of anti-MMP9 antibodies of the present disclosure are described in more detail below.

Anti-MMP9 antibodies can be described in terms of the CDRs of the heavy and light chains. In some embodiments, an antibody is a humanized antibody or a human antibody. Humanized antibodies include human immunoglobulins (recipient antibody) in which residues from a complementary-determining region (CDR) of the recipient are replaced by residues from a CDR of a non-human species (donor antibody) such as mouse, rat or rabbit having the desired specificity, affinity and capacity. Thus, humanized forms of non-human (e.g., murine) antibodies are chimeric immunoglobulins which contain minimal sequence derived from non-human immunoglobulin. The non-human sequences are located primarily in the variable regions, particularly in the complementarity-determining regions (CDRs). In some embodiments, Fv framework residues of the human immunoglobulin are replaced by corresponding non-human residues. Humanized antibodies can also comprise residues that are found neither in the recipient antibody nor in the imported CDR or framework sequences. In certain embodiments, a humanized antibody comprises substantially all of at least one, and typically two, variable domains, in which all or substantially all of the CDRs correspond to those of a non-human immunoglobulin and all or substantially all of the framework regions are those of a human immunoglobulin consensus sequence. For the purposes of the present disclosure, humanized antibodies can also include immunoglobulin fragments, such as Fv, Fab, Fab′, F(ab′)₂ or other antigen-binding subsequences of antibodies.

The humanized antibody can also comprise at least a portion of an immunoglobulin constant region (Fc), typically that of a human immunoglobulin. See, for example, Jones et al. (1986) Nature 321:522-525; Riechmann et al. (1988) Nature 332:323-329; and Presta (1992) Curr. Op. Struct. Biol. 2:593-596.

Methods for humanizing non-human antibodies are known in the art. Generally, a humanized antibody has one or more amino acid residues introduced into it from a source that is non-human. These non-human amino acid residues are often referred to as “import” or “donor” residues, which are typically obtained from an “import” or “donor” variable domain. For example, humanization can be performed essentially according to the method of Winter and co-workers, by substituting rodent CDRs or CDR sequences for the corresponding sequences of a human antibody. See, for example, Jones et al., supra; Riechmann et al., supra and Verhoeyen et al. (1988) Science 239:1534-1536. Accordingly, such “humanized” antibodies include chimeric antibodies (U.S. Pat. No. 4,816,567), wherein substantially less than an intact human variable domain has been substituted by the corresponding sequence from a non-human species. In certain embodiments, humanized antibodies are human antibodies in which some CDR residues and optionally some framework region residues are substituted by residues from analogous sites in rodent antibodies (e.g., murine monoclonal antibodies).

Human antibodies can also be produced, for example, by using phage display libraries. Hoogenboom et al. (1991) J. Mol. Biol, 227:381; Marks et al. (1991) J. Mol. Biol. 222:581. Other methods for preparing human monoclonal antibodies are described by Cole et al. (1985) “Monoclonal Antibodies and Cancer Therapy,” Alan R. Liss, p. 77 and Boerner et al. (1991) J. Immunol. 147:86-95.

Human antibodies can be made by introducing human immunoglobulin loci into transgenic animals (e.g., mice) in which the endogenous immunoglobulin genes have been partially or completely inactivated. Upon immunological challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al. (1992) Bio/Technology 10:779-783 (1992); Lonberg et al. (1994) Nature 368: 856-859; Morrison (1994) Nature 368:812-813; Fishwald et al. (1996) Nature Biotechnology 14:845-851; Neuberger (1996) Nature Biotechnology 14:826; and Lonberg et al. (1995) Intern. Rev. Immunol. 13:65-93.

Antibodies can be affinity matured using known selection and/or mutagenesis methods as described above. In some embodiments, affinity matured antibodies have an affinity which is five times or more, ten times or more, twenty times or more, or thirty times or more than that of the starting antibody (generally murine, rabbit, chicken, humanized or human) from which the matured antibody is prepared.

An antibody can also be a bispecific antibody. Bispecific antibodies are monoclonal, and may be human or humanized antibodies that have binding specificities for at least two different antigens. In the present case, the two different binding specificities can be directed to two different MMPs, or to two different epitopes on a single MMP (e.g., MMP9).

An antibody as disclosed herein can also be an immunoconjugate. Such immunoconjugates comprise an antibody (e.g., to MMP9) conjugated to a second molecule, such as a reporter. An immunoconjugate can also comprise an antibody conjugated to a cytotoxic agent such as a chemotherapeutic agent, a toxin (e.g., an enzymatically active toxin of bacterial, fungal, plant, or animal origin, or fragments thereof), or a radioactive isotope (i.e., a radioconjugate).

An antibody that “specifically binds to” or is “specific for” a particular polypeptide or an epitope on a particular polypeptide is one that binds to that particular polypeptide or epitope without substantially binding to any other polypeptide or polypeptide epitope. In some embodiments, an antibody of the present disclosure specifically binds to human MMP9 with a dissociation constant (K_{d}) equal to or lower than 100 nM, optionally lower than 10 nM, optionally lower than 1 nM, optionally lower than 0.5 nM, optionally lower than 0.1 nM, optionally lower than 0.01 nM, or optionally lower than 0.005 nM; in the form of monoclonal antibody, scFv, Fab, or other form of antibody measured at a temperature of about 4° C., 25° C., 37° C. or 42° C.

In certain embodiments, use of an antibody of the present disclosure binds to one or more processing sites (e.g., sites of proteolytic cleavage) in MMP9, thereby effectively blocking processing of the proenzyme or preproenzyme to the catalytically active enzyme, and thus reducing the proteolytic activity of the MMP9. In certain embodiments, use of an antibody according to the present disclosure binds to MMP9 with an affinity at least 2 times, at least 5 times, at least 10 times, at least 25 times, at least 50 times, at least 100 times, at least 500 times, or at least 1000 times greater than its binding affinity for another MMP. Binding affinity can be measured by any method known in the art and can be expressed as, for example, on-rate, off-rate, dissociation constant (K_{d}), equilibrium constant (K_{eq}) or any term in the art.

In certain embodiments, use of an antibody according to the present disclosure is a non-competitive inhibitor of the catalytic activity of MMP9. In certain embodiments, an antibody according to the present disclosure binds within the catalytic domain of MMP9. In additional embodiments, an antibody according to the present disclosure binds outside the catalytic domain of MMP9.

The present disclosure also contemplates use in the methods, regimens, kits, and articles of manufacture herein of antibodies, or antigen binding fragments thereof, that compete with anti-MMP9 antibodies or antigen binding fragments thereof described herein for binding to MMP9. Thus, the present disclosure contemplates use of anti-MMP9 antibodies, and functional fragments thereof, that compete for binding with, for example, an antibody having a heavy chain polypeptide of any of SEQ ID NOS:1 or 5-8, a light chain polypeptide of SEQ ID NOS:2 or 9-12, or combinations thereof. In one embodiment, the anti-MMP9 antibody, for functional fragment thereof, competes for binding to human MMP9 with the antibody described herein as AB0041.

### MMP9 Sequence

The amino acid sequence of human MMP9 protein is as follows:

| (SEQ ID NO: 27) | |
|---|---|
| MSLWQPLVLV LLVLGCCFAA PRQRQSTLVL FPGDLRTNLT DRQLAEEYLY | 50 |
| RYGYTRVAEM RGESKSLGPA LLLLQKQLSL PETGELDSAT LKAMRTPRCG | 100 |
| VPDLGRFQTF EGDLKWHHHN ITYWIQNYSE DLPRAVIDDA FARAFALWSA | 150 |
| VTPLTFTRVY SRDADIVIQF GVAEHGDGYP FDGKDGLLAH AFPPGPGIQG | 200 |
| DAHFDDDELW SLGKGVVVPT RFGNADGAAC HFPFIFEGRS YSACTTDGRS | 250 |
| DGLPWCSTTA NYDTDDRFGF CPSERLYTRD GNADGKPCQF PFIFQGQSYS | 300 |
| ACTIDGRSDG YRWCATTANY DRDKLFGFCP TRADSTVMGG NSAGELCVFP | 350 |
| FTFLGKEYST CTSEGRGDGR LWCATTSNFD SDKKWGFCPD QGYSLFLVAA | 400 |
| HEFGHALGLD HSSVPEALMY PMYRFTEGPP LHKDDVNGIR HLYGPRPEPE | 450 |
| PRPPTTTTPQ PTAPPTVCPT GPPTVHPSER PTAGPTGPPS AGPTGPPTAG | 500 |
| PSTATTVPLS PVDDACNVNI FDAIAEIGNQ LYLFKDGKYW RFSEGRGSRP | 550 |
| QGPFLIADKW PALPRKLDSV FEEPLSKKLF FFSGRQVWVY TGASVLGPRR | 600 |
| LDKLGLGADV AQVTGALRSG RGKMLLFSGR RLWRFDVKAQ MVDPRSASEV | 650 |
| DRMFPGVPLD THDVFQYREK AYFCQDRFYW RVSSRSELNQ VDQVGYVTYD | 700 |
| ILQCPED | |

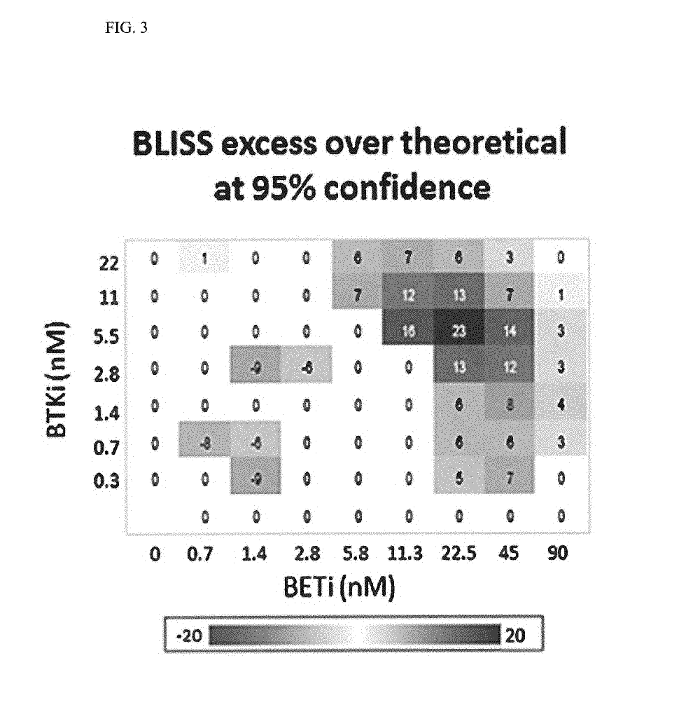

Protein domains are shown schematically in FIG. 3 and are indicated below:

| Amino Acid # | Feature |
|---|---|
| 1-19 | Signal Peptide |
| 38-98 | Peptidoglycan Binding Domain |
| R98/C99 | Propetide cleavage site (dependent on cleavage enzyme) |
| 112-445 | Zn dependent metalloproteinase domain |
| 223-271 | Fibronectin type II domain (gelatin binding domain) |
| 281-329 | Fibronectin type II domain (gelatin binding domain) |
| 340-388 | Fibronectin type II domain (gelatin binding domain) |
| 400-411 | Zn binding region |
| 521-565 | Hemopexin-like domain |
| 567-608 | Hemopexin-like domain |
| 613-659 | Hemopexin-like domain |
| 661-704 | Hemopexin-like domain |

The amino acid sequence of mature full-length human MMP9 (which is the amino acid sequence of the propolypeptide of SEQ ID NO:27 without the signal peptide) is:

(SEQ ID NO: 28)
PRQRQSTLVL FPGDLRTNLT DRQLAEEYLY RYGYTRVAEM
RGESKSLGPA LLLLQKQLSL PETGELDSAT LKAMRTPRCG
VPDLGRFQTF EGDLKWHHHN ITYWIQNYSE DLPRAVIDDA
FARAFALWSA VTPLTFTRVY SRDADIVIQF GVAFHGDGYP
FDGKDGLLAH AFPPGPGIQG DAHFDDDELW SLGKGVVVPT
RFGNADGAAC HFPFIFEGRS YSACTTDGRS DGLPWCSTTA
NYDTDDRFGF CPSERLYTRD GNADGKPCQF PFIFQGQSYS
ACTTDGRSDG YRWCATTANY DRDKLFGFCP TRADSTVMGG
NSAGELCVFP FTFLGKEYST CTSEGRGDGR LWCATTSNFD
SDKKWGFCPD QGYSLFLVAA HEFGHALGLD HSSVPEALMY
PMYRFTEGPP LHKDDVNGIR HLYGPRPEPE PRPPTTTTPQ
PTAPPTVCPT GPPTVHPSER PTAGPTGPPS AGPTGPPTAG
PSTATTVPLS PVDDACNVNI FDAIAEIGNQ LYLFKDGKYW
RFSEGRGSRP QGPFLIADKW PALPRKLDSV FEEPLSKKLF
FFSGRQVWVY TGASVLGPRR LDKLGLGADV AQVTGALRSG
RGKMLLFSGR RLWRFDVKAQ MVDPRSASEV DRMFPGVPLD
THDVFQYREK AYFCQDRFYW RVSSRSELNQ VDQVGYVTYD
ILQCPED

The amino acid sequence of the signal peptide is MSLWQPLVLV LLVLGCCFAA (SEQ ID NO:29).

The present disclosure contemplate the use of MMP9 binding proteins that bind any portion of MMP9, e.g., human MMP9, with MMP9 binding proteins that preferentially bind MMP9 relative to other MMPs being of particular interest. Anti-MMP9 antibodies, and functional fragments thereof, can be generated accordingly to methods well known in the art. Examples of anti-MMP9 antibodies are provided below.

### Mouse Monoclonal Anti-MMP9

A mouse monoclonal antibody to human MMP9 was obtained as described in Example 2. This antibody contains a mouse IgG2b heavy chain and a mouse kappa light chain, and is denoted AB0041.

The amino acid sequence of the AB0041 heavy chain is as follows:

(SEQ ID NO: 1)
MAVLVLFLCLVAFPSCVLSQVQLKESGPGLVAPSQSLSITCTVSGF
SLLSYGVHWVRQPPGKGLEWLGVIWTGGTTNYNSALMSRLSISKDD
SKSQVFLKMNSLQTDDTAIYYCARYYYGMDYWGQGTSVTVSSAKTT
PPSVYPLAPGCGDTTGSSVTLGCLVKGYFPESVTVTWNSGSLSSSV
HTFPALLQSGLYTMSSSVTVPSSTWPSQTVTCSVAHPASSTTVDKK
LEPSGPISTINPCPPCKECHKCPAPNLEGGPSVFIFPPNIKDVLMI
SLTPKVTCVVVDVSEDDPDVRISWFVNNVEVHTAQTQTHREDYNST
IRVVSALPIQHQDWMSGKEFKCKVNNKDLPSPIERTISKIKGLVRA
PQVYILPPPAEQLSRKDVSLTCLVVGFNPGDISVEWTSNGHTEENY
KDTAPVLDSDGSYFIYSKLDIKTSKWEKTDSFSCNVRHEGLKNYYL
KKTISRSPGK

The signal sequence is underlined, and the sequence of the IgG2b constant region is presented italics.

The amino acid sequence of theAB0041 light chain is as follows:

(SEQ ID NO: 2)
MESQIQVFVFVFLWLSGVDGDIVMTQSHKFMSTSVGDRVSITCKAS
QDVRNTVAWYQQKTGQSPKLLIYSSSYRNTGVPDRFTGSGSGTDFT
FTISSVQAEDLAVYFCQQHYITPYTFGGGTKLEIKRADAAPTVSIF
PPSSEQLTSGGASVVCFLNNFYPKDINVKWKIDGSERQNGVLNSWT
DQDSKDSTYSMSSTLTLTKDEYERHNSYTCEATHKTSTSPIVKSFN
RNEC

The signal sequence is underlined, and the sequence of the kappa constant region is presented in italics.

The following amino acid sequence comprises the framework regions and complementarity-determining regions (CDRs) of the variable region of the IgG2b heavy chain of AB0041 (with CDRs underlined):

(SEQ ID NO: 3)
QVQLKESGPGLVAPSQSLSITCTVSGFSLLSYGVHWVRQPPGKGLE
WLGVIWTGGTTNYNSALMSRLSISKDDSKSQVFLKMNSLQTDDTAI
YYCARYYYGMDYWGQGTSVTVSS

The following amino acid sequence comprises the framework regions and complementarity-determining regions (CDRs) of the variable region of the kappa light chain of AB0041 (with CDRs underlined):

(SEQ ID NO: 4)
DIVMTQSHKFMSTSVGDRVSITCKASQDVRNTVAWYQQKTGQSPKL
LIYSSSYRNTGVPDRFTGSGSGTDFTFTISSVQAEDLAVYFCQQHY
ITPYTFGGGTKLEIK

### Heavy-Chain Variants

As noted in U.S. Pat. No. 8,377,443 (McAuley et al.), 8,501,916 (McAuley et al.), and 9,120,863 (McAuley et al.), the amino acid sequences of the variable regions of the AB0041 heavy and light chains were separately modified, by altering framework region sequences in the heavy and light chain variable regions. The effect of these sequence alterations was to deplete the antibody of human T-cell epitopes, thereby reducing or abolishing its immunogenicity in humans (Antitope, Babraham, UK).

Four heavy-chain variants were constructed, in a human IgG4 heavy chain background containing a S241P amino acid change that stabilizes the hinge domain (Angal et al. (1993) Molec. Immunol. 30:105-108), and are denoted VH1, VH2, VH3 and VH4. The amino acid sequences of their framework regions and CDRs are as follows:

VH1
(SEQ ID NO: 5
QVQLQESGPGLVKPSETLSLTCTVSGFSLLSYGVHWVRQPPGKGLE
WLGVIWTGGTTNYNSALMSRLTISKDDSKSTVYLKMNSLKTEDTAI
YYCARYYYGMDYWGQGTSVTVSS
VH2
(SEQ ID NO: 6)
QVQLQESGPGLVKPSETLSLTCTVSGFSLLSYGVHWVRQPPGKGLE
WLGVIWTGGTTNYNSALMSRLTISKDDSKNTVYLKMNSLKTEDTAI
YYCARYYYGMDYWGQGTINTVSS
VH3
(SEQ ID NO: 7)
QVQLQESGPGLVKPSETLSLTCTVSGFSLLSYGVHWVRQPPGKGLE
WLGVIWTGGTTNYNSALMSRFTISKDDSKNTVYLKMNSLKTEDTAI
YYCARYYYGMDYWGQGTLVTVSS
VH4
(SEQ ID NO: 8)
QVQLQESGPGLVKPSETLSLTCTVSGTSLLSYGVHWVRQPPGKGLE
WLGVIWTGGTTNYNSALMSRFTISKDDSKNTLYLKMNSLKTEDTAI
YYCARYYYGMDYWGQGTINTVSS

### Light-Chain Variants

Four light-chain variants were constructed, in a human kappa chain background, and are denoted Vk1, Vk2, Vk3 and Vk4. The amino acid sequences of their framework regions and CDRs are as follows:

Vk1
(SEQ ID NO: 9)
DIVMTQSPSFLSASVGDRVTITCKASQDVRNTVAWYQQKTGKAPKL
LIYSSSYRNTGVPDRFTGSGSGTDFTLTISSLQAEDVAVYFCQQHY
ITPYTFGGGTKVEIK
Vk2
(SEQ ID NO: 10)
DIVMTQSPSSLSASVGDRVTITCKASQDVRNTVAWYQQKPGKAPKL
LIYSSSYRNTGVPDRFTGSGSGTDFTLTISSLQAEDVAVYFCQQHY
ITPYTFGGGTKVEIK
Vk3
(SEQ ID NO: 11)
DIQMTQSPSSLSASVGDRVTITCKASQDVRNTVAWYQQKPGKAPKL
LIYSSSYRNTGVPDRFSGSGSGTDFTLTISSLQAEDVAVYFCQQHY
ITPYTFGGGTKVEIK
Vk4
(SEQ ID NO: 12)
DIQMTQSPSSLSASVGDRVTITCKASQDVRNTVAWYQQKPGKAPKL
LIYSSSYRNTGVPDRFSGSGSGTDFTLTISSLQAEDVAVYYCQQHY
ITPYTFGGGTKVEIK

The humanized heavy and light chains are combined in all possible pairwise combinations to generate a number of functional humanized anti-MMP9 antibodies.

Additional heavy chain variable region amino acid sequences having 75% or more, 80% or more, 90% or more, 95% or more, or 99% or more homology to the heavy chain variable region sequences disclosed herein are also provided. Furthermore, additional light chain variable region amino acid sequences having 75% or more, 80% or more, 90% or more, 95% or more, or 99% or more homology to the light chain variable region sequences disclosed herein are also provided.

Additional heavy chain variable region amino acid sequences having 75% or more, 80% or more, 90% or more, 95% or more, or 99% or more sequence identity to the heavy chain variable region sequences disclosed herein are also provided. Furthermore, additional light chain variable region amino acid sequences having 75% or more, 80% or more, 90% or more, 95% or more, or 99% or more sequence identity to the light chain variable region sequences disclosed herein are also provided.

### Complementarity-Determining Regions (CDRs)

The CDRs of the heavy chain of an anti-MMP9 antibody as disclosed herein have the following amino acid sequences:

CDR1:
(SEQ ID NO: 13)
GFSLLSYGVH
CDR2:
(SEQ ID NO: 14)
VIWTGGTTNYNSALMS
CDR3:
(SEQ ID NO: 15)
YYYGMDY

The CDRs of the light chain of an anti-MMP9 antibody as disclosed herein have the following amino acid sequences:

CDR1:
(SEQ ID NO: 16)
KASQDVRNTVA
CDR2:
(SEQ ID NO: 17)
SSSYRNT
CDR3:
(SEQ ID NO: 18)
QQHYITPYT

### Nucleic Acids Encoding Anti-MMP9 Antibodies

The present disclosure provides use in the methods, regimens, kits, and articles of manufacture herein of nucleic acids encoding anti-MMP9 antibodies and functional fragments thereof. Accordingly, the present disclosure provides an isolated polynucleotide (nucleic acid) encoding an antibody or antigen-binding fragment as described herein, vectors containing such polynucleotides, and host cells and expression systems for transcribing and translating such polynucleotides into polypeptides. The present disclosure also contemplates the use of constructs in the form of plasmids, vectors, transcription or expression cassettes which comprise at least one polynucleotide as above.

The present disclosure also provides the use of a recombinant host cell which comprises one or more constructs as above, as well as methods of production of the antibody or antigen-binding fragments thereof described herein which method comprises expression of nucleic acid encoding a heavy chain polypeptide and a light chain polypeptide (in the same or different host cells, and from the same or different constructs) in a recombination host cell. Expression can be achieved by culturing under appropriate conditions recombinant host cells containing the nucleic acid. Following production by expression, an antibody or antigen-binding fragment can be isolated and/or purified using any suitable technique, then used as appropriate.

Systems for cloning and expression of a polypeptide in a variety of different host cells are well known. Suitable host cells include bacteria, mammalian cells, yeast, and baculovirus systems. Mammalian cell lines available in the art for expression of a heterologous polypeptide include Chinese hamster ovary cells, HeLa cells, baby hamster kidney cells, NSO mouse melanoma cells and many others. A common bacterial host is E. coli.

Suitable vectors can be chosen or constructed, containing appropriate regulatory sequences, including operably linked promoter sequences, terminator sequences, polyadenylation sequences, enhancer sequences, marker genes and/or other sequences as appropriate. Vectors can be plasmids, viral e.g. ‘phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Short Protocols in Molecular Biology, Second Edition, Ausubel et al. eds., John Wiley & Sons, 1992. The disclosures of Sambrook et al. and Ausubel et al. are incorporated herein by reference in their entirety.

The nucleic acid encoding a polypeptide of interest is integrated into the genome of the host cell or can be maintained as a stable or transient episomal element. Any of a wide variety of expression control sequences—sequences that control the expression of a DNA sequence operatively linked to it—can be used in these vectors to express the DNA sequences. For example, a nucleic acid encoding a polypeptide of interest can be operably linked to a promoter, and provided in an expression construct for use in methods of production of recombinant MMP9 proteins or portions thereof. Those of skill in the art are aware that nucleic acids encoding the antibody chains disclosed herein can be synthesized using standard knowledge and procedures in molecular biology.

Examples of nucleotide sequences encoding the heavy and light chain amino acid sequences disclosed herein, are as follows:

VH1:
(SEQ ID NO: 19)
CAGGTGCAGC TGCAGGAATC CGGCCCTGGC
CTGGTCAAGC CCTCCGAGAC ACTGTCCCTG ACCTGCACCG
TGTCCGGCTT CTCCCTGCTG TCCTACGGCG TGCACTGGGT
CCGACAGCCT CCAGGGAAGG GCCTGGAATG GCTGGGCGTG
ATCTGGACCG GCGGCACCAC CAACTACAAC TCCGCCCTGA
TGTCCCGGCT GACCATCTCC AAGGACGACT CCAAGTCCAC
CGTGTACCTG AAGATGAACT CCCTGAAAAC CGAGGACACC
GCCATCTACT ACTGCGCCCG GTACTACTAC GGCATGGACT
ACTGGGGCCA GGGCACCTCC GTGACCGTGT CCTCA
VH2:
(SEQ ID NO: 20)
CAGGTGCAGC TGCAGGAATC CGGCCCTGGC
CTGGTCAAGC CCTCCGAGAC ACTGTCCCTG ACCTGCACCG
TGTCCGGCTT CTCCCTGCTG TCCTACGGCG TGCACTGGGT
CCGACAGCCT CCAGGCAAAG GCCTGGAATG GCTGGGCGTG
ATCTGGACCG GCGGCACCAC CAACTACAAC TCCGCCCTGA
TGTCCCGGCT GACCATCTCC AAGGACGACT CCAAGAACAC
CGTGTACCTG AAGATGAACT CCCTGAAAAC CGAGGACACC
GCCATCTACT ACTGCGCCCG GTACTACTAC GGCATGGACT
ACTGGGGCCA GGGCACCCTG GTCACCGTGT CCTCA
VH3:
(SEQ ID NO: 21)
CAGGTGCAGC TGCAGGAATC CGGCCCTGGC
CTGGTCAAGC CCTCCGAGAC ACTGTCCCTG ACCTGCACCG
TGTCCGGCTT CTCCCTGCTG TCCTACGGCG TGCACTGGGT
CCGACAGCCT CCAGGCAAAG GCCTGGAATG GCTGGGCGTG
ATCTGGACCG GCGGCACCAC CAACTACAAC TCCGCCCTGA
TGTCCCGGTT CACCATCTCC AAGGACGACT CCAAGAACAC
CGTGTACCTG AAGATGAACT CCCTGAAAAC CGAGGACACC
GCCATCTACT ACTGCGCCCG GTACTACTAC GGCATGGACT
ACTGGGGCCA GGGCACCCTG GTCACCGTGT CCTCA
VH4:
(SEQ ID NO: 22)
CAGGTGCAGC TGCAGGAATC CGGCCCTGGC
CTGGTCAAGC CCTCCGAGAC ACTGTCCCTG ACCTGCACCG
TGTCCGGCTT CTCCCTGCTG TCCTACGGCG TGCACTGGGT
CCGACAGCCT CCAGGCAAAG GCCTGGAATG GCTGGGCGTG
ATCTGGACCG GCGGCACCAC CAACTACAAC TCCGCCCTGA
TGTCCCGGTT CACCATCTCC AAGGACGACT CCAAGAACAC
CCTGTACCTG AAGATGAACT CCCTGAAAAC CGAGGACACC
GCCATCTACT ACTGCGCCCG GTACTACTAC GGCATGGACT
ACTGGGGCCA GGGCACCCTG GTCACCGTGT CCTCA
Vk1:
(SEQ ID NO: 23)
GACATCGTGA TGACCCAGTC CCCCAGCTTC
CTGTCCGCCT CCGTGGGCGA CAGAGTGACC ATCACATGCA
AGGCCTCTCA GGACGTGCGG AACACCGTGG CCTGGTATCA
GCAGAAAACC GGCAAGGCCC CCAAGCTGCT GATCTACTCC
TCCTCCTACC GGAACACCGG CGTGCCCGAC CGGTTTACCG
GCTCTGGCTC CGGCACCGAC TTTACCCTGA CCATCAGCTC
CCTGCAGGCC GAGGACGTGG CCGTGTACTT CTGCCAGCAG
CACTACATCA CCCCCTACAC CTTCGGCGGA GGCACCAAGG
TGGAAATAAA A
Vk2:
(SEQ ID NO: 24)
GACATCGTGA TGACCCAGTC CCCCTCCAGC
CTGTCCGCCT CTGTGGGCGA CAGAGTGACC ATCACATGCA
AGGCCTCTCA GGACGTGCGG AACACCGTGG CCTGGTATCA
GCAGAAGCCC GGCAAGGCCC CCAAGCTGCT GATCTACTCC
TCCTCCTACC GGAACACCGG CGTGCCCGAC CGGTTTACCG
GCTCTGGCTC CGGCACCGAC TTTACCCTGA CCATCAGCTC
CCTGCAGGCC GAGGACGTGG CCGTGTACTT CTGCCAGCAG
CACTACATCA CCCCCTACAC CTTCGGCGGA GGCACCAAGG
TGGAAATAAA A
Vk3:
(SEQ ID NO: 25)
GACATCCAGA TGACCCAGTC CCCCTCCAGC
CTGTCCGCCT CTGTGGGCGA CAGAGTGACC ATCACATGCA
AGGCCTCCCA GGACGTGCGG AACACCGTGG CCTGGTATCA
GCAGAAGCCC GGCAAGGCCC CCAAGCTGCT GATCTACTCC
TCCTCCTACC GGAACACCGG CGTGCCCGAC CGGTTCTCTG
GCTCTGGAAG CGGCACCGAC TTTACCCTGA CCATCAGCTC
CCTGCAGGCC GAGGACGTGG CCGTGTACTT CTGCCAGCAG
CACTACATCA CCCCCTACAC CTTCGGCGGA GGCACCAAGG
TGGAAATAAA A
Vk4:
(SEQ ID NO: 26)
GACATCCAGA TGACCCAGTC CCCCTCCAGC
CTGTCCGCCT CTGTGGGCGA CAGAGTGACC ATCACATGCA
AGGCCTCTCA GGACGTGCGG AACACCGTGG CCTGGTATCA
GCAGAAGCCC GGCAAGGCCC CCAAGCTGCT GATCTACTCC
TCCTCCTACC GGAACACCGG CGTGCCCGAC CGGTTCTCTG
GCTCTGGAAG CGGCACCGAC TTTACCCTGA CCATCAGCTC
CCTGCAGGCC GAGGACGTGG CCGTGTACTA CTGCCAGCAG
CACTACATCA CCCCCTACAC CTTCGGCGGA GGCACCAAGG
TGGAAATAAA A

Because the structure of antibodies, including the juxtaposition of CDRs and framework regions in the variable region, the structure of framework regions and the structure of heavy- and light-chain constant regions, is well-known in the art; it is well within the skill of the art to obtain related nucleic acids that encode anti-MMP-9 antibodies. Accordingly, polynucleotides comprising nucleic acid sequences having at least 75%, at least 80%, at least 85%, at least 90%, at least 95%, at least 98% and at least 99% homology to any of the nucleotide sequences disclosed herein are also provided. Accordingly, polynucleotides comprising nucleic acid sequences having at least 75%, at least 80%, at least 85%, at least 90%, at least 95%, at least 98% and at least 99% identity to any of the nucleotide sequences disclosed herein are also provided.

MMP9 binding proteins, as well as nucleic acid (e.g., DNA or RNA) encoding MMP9 binding proteins, can be provided as a pharmaceutical composition, e.g., combined with a pharmaceutically acceptable carrier or excipient. Such pharmaceutical compositions are useful for, for example, administration to a subject in vivo or ex vivo, and for diagnosing and/or treating a subject with the MMP9 binding proteins.

Pharmaceutically acceptable carriers are physiologically acceptable to the administered patient and retain the therapeutic properties of the antibodies or peptides with which it is administered. Pharmaceutically-acceptable carriers and their formulations are and generally described in, for example, Remington' pharmaceutical Sciences (18th Edition, ed. A. Gennaro, Mack Publishing Co., Easton, Pa. 1990). One exemplary pharmaceutical carrier is physiological saline. Each carrier is “pharmaceutically acceptable” in the sense of being compatible with the other ingredients of the formulation and not substantially injurious to the patient.

Pharmaceutical compositions can be formulated to be compatible with a particular route of administration, systemic or local. Thus, pharmaceutical compositions include carriers, diluents, or excipients suitable for administration by various routes.

Pharmaceutical compositions can include pharmaceutically acceptable additives. Examples of additives include, but are not limited to, a sugar such as mannitol, sorbitol, glucose, xylitol, trehalose, sorbose, sucrose, galactose, dextran, dextrose, fructose, lactose and mixtures thereof. Pharmaceutically acceptable additives can be combined with pharmaceutically acceptable carriers and/or excipients such as dextrose. Additives also include surfactants such as polysorbate 20 or polysorbate 80.

The formulation and delivery methods will generally be adapted according to the site and the disease to be treated. Exemplary formulations include, but are not limited to, those suitable for parenteral administration, e.g., intravenous, intra-arterial, intramuscular, or subcutaneous administration.

Pharmaceutical compositions for parenteral delivery include, for example, water, saline, phosphate buffered saline, Hank's solution, Ringer's solution, dextrose/saline, and glucose solutions. The formulations can contain auxiliary substances to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate. Additional parenteral formulations and methods are described in Bai (1997) J. Neuroimmunol. 80:65 75; Warren (1997) J. Neurol. Sci. 152:31 38; and Tonegawa (1997) J. Exp. Med. 186:507 515. The parenteral preparation can be enclosed in ampules, disposable syringes or multiple dose vials made of glass or plastic.

Pharmaceutical compositions for intradermal or subcutaneous administration can include a sterile diluent, such as water, saline solution, fixed oils, polyethylene glycols, glycerine, propylene glycol or other synthetic solvents; antibacterial agents such as benzyl alcohol or methyl parabens; antioxidants such as ascorbic acid, glutathione or sodium bisulfite; chelating agents such as ethylenediaminetetraacetic acid; buffers such as acetates, citrates or phosphates and agents for the adjustment of tonicity such as sodium chloride or dextrose.

Pharmaceutical compositions for injection include aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). The carrier can be a solvent or dispersion medium containing, for example, water, ethanol, polyol (for example, glycerol, propylene glycol, and liquid polyetheylene glycol, and the like), and suitable mixtures thereof. Fluidity can be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. Isotonic agents, for example, sugars, polyalcohols such as manitol, sorbitol, and sodium chloride may be included in the composition. The resulting solutions can be packaged for use as is, or lyophilized; the lyophilized preparation can later be combined with a sterile solution prior to administration.

Pharmaceutically acceptable carriers can contain a compound that stabilizes, increases or delays absorption or clearance. Such compounds include, for example, carbohydrates, such as glucose, sucrose, or dextrans; low molecular weight proteins; compositions that reduce the clearance or hydrolysis of peptides; or excipients or other stabilizers and/or buffers. Agents that delay absorption include, for example, aluminum monostearate and gelatin. Detergents can also be used to stabilize or to increase or decrease the absorption of the pharmaceutical composition, including liposomal carriers. To protect from digestion the compound can be complexed with a composition to render it resistant to acidic and enzymatic hydrolysis, or the compound can be complexed in an appropriately resistant carrier such as a liposome. Means of protecting compounds from digestion are known in the art (see, e.g., Fix (1996) Pharm Res. 13:1760 1764; Samanen (1996) J. Pharm. Pharmacol. 48:119 135; and U.S. Pat. No. 5,391,377, describing lipid compositions for oral delivery of therapeutic agents).

Compositions of the present invention can be combined with other therapeutic moieties or imaging/diagnostic moieties as provided herein. Therapeutic moieties and/or imaging moieties can be provided as a separate composition, or as a conjugated moiety present on an MMP9 binding protein.

Formulations for in vivo administration are generally sterile. In one embodiment, the pharmaceutical compositions are formulated to be free of pyrogens such that they are acceptable for administration to human patients.

Various other pharmaceutical compositions and techniques for their preparation and use will be known to those of skill in the art in light of the present disclosure. For a detailed listing of suitable pharmacological compositions and associated administrative techniques one can refer to the detailed teachings herein, which can be further supplemented by texts such as Remington: The Science and Practice of Pharmacy 20th Ed. (Lippincott, Williams & Wilkins 2003).

Pharmaceutical compositions can be formulated based on the physical characteristics of the patient/subject needing treatment, the route of administration, and the like. Such can be packaged in a suitable pharmaceutical package with appropriate labels for the distribution to hospitals and clinics wherein the label is for the indication of treating a disorder as described herein in a subject. Medicaments can be packaged as a single or multiple units. Instructions for the dosage and administration of the pharmaceutical compositions of the present invention can be included with the pharmaceutical packages and kits described below.

### Methods of Use and Treatments

The methods disclosed herein may be used for treating cancer in a human in need thereof, comprising administering to the human a therapeutically effective amount of a BTK inhibitor in combination with one or more inhibitor. For example, the one or more inhibitor may be therapeutically effective amounts of a JAK inhibitor, an ASK1 inhibitor, a BET inhibitor and a MMP9 inhibitor, as described herein.

The method of use or treatment described herein may comprise Compound A1, or a pharmaceutically acceptable salt or hydrate thereof, in combination with one or more inhibitor and another pharmaceutical or therapeutic agent. In each of the methods described herein, pharmaceutically effective amounts of each inhibitor and each pharmaceutical agent are used.

### Diseases

In some aspects, the disease or condition is chosen from an autoimmune disease, an inflammatory disease, a neurodegenerative disease, a cardiovascular disorder, a renal disorder, a viral infection, and obesity. In some aspects, the disease or condition is chosen from rheumatoid arthritis, osteoarthritis, atherosclerosis, psoriasis, systemic lupus erythematosus, multiple sclerosis, inflammatory bowel disease, asthma, chronic obstructive airways disease, pneumonitis, dermatitis, alopecia, nephritis, vasculitis, atherosclerosis, Alzheimer's disease, hepatitis, primary biliary cirrhosis, sclerosing cholangitis, diabetes (including type I diabetes), and acute rejection of transplanted organs. In some aspects the disease or condition is cancer, including hematological cancers, lymphoma, multiple myelomas, leukemias, a neoplasm, cancer or tumor (for example a solid tumor).

In some embodiments, the cancer is carcinoma, sarcoma, melanoma, lymphoma or leukemia. In other embodiments, the cancer is a hematologic malignancy. In some embodiments, the cancer is leukemia (e.g., chronic lymphocytic leukemia), lymphoma (e.g., non-Hodgkin's lymphoma), or multiple myeloma. In other embodiments, the cancer is a solid tumor.

In some variations, the cancer is small lymphocytic lymphoma, non-Hodgkin's lymphoma, indolent non-Hodgkin's lymphoma (iNHL), refractory iNHL, mantle cell lymphoma, follicular lymphoma, lymphoplasmacytic lymphoma, marginal zone lymphoma, immunoblastic large cell lymphoma, lymphoblastic lymphoma, Splenic marginal zone B-cell lymphoma (+/−villous lymphocytes), nodal marginal zone lymphoma (+/−monocytoid B-cells), extranodal marginal zone B-cell lymphoma of mucosa-associated lymphoid tissue type, cutaneous T-cell lymphoma, extranodal T-cell lymphoma, anaplastic large cell lymphoma, angioimmunoblastic T-cell lymphoma, mycosis fungoides, B-cell lymphoma, diffuse large B-cell lymphoma, Mediastinal large B-cell lymphoma, Intravascular large B-cell lymphoma, Primary effusion lymphoma, small non-cleaved cell lymphoma, Burkitt's lymphoma, multiple myeloma, plasmacytoma, acute lymphocytic leukemia, T-cell acute lymphoblastic leukemia, B-cell acute lymphoblastic leukemia, B-cell prolymphocytic leukemia, acute myeloid leukemia, chronic lymphocytic leukemia, juvenile myelomonocytic leukemia, minimal residual disease, hairy cell leukemia, primary myelofibrosis, secondary myelofibrosis, chronic myeloid leukemia, myelodysplastic syndrome, myeloproliferative disease, or Waldestrom's macroglobulinemia.

In other variations, the cancer is pancreatic cancer, urological cancer, bladder cancer, colorectal cancer, colon cancer, breast cancer, prostate cancer, renal cancer, hepatocellular cancer, thyroid cancer, gall bladder cancer, lung cancer (e.g. non-small cell lung cancer, small-cell lung cancer), ovarian cancer, cervical cancer, gastric cancer, endometrial cancer, esophageal cancer, head and neck cancer, melanoma, neuroendocrine cancer, CNS cancer, brain tumors (e.g., glioma, anaplastic oligodendroglioma, adult glioblastoma multiforme, and adult anaplastic astrocytoma), bone cancer, soft tissue sarcoma, retinoblastomas, neuroblastomas, peritoneal effusions, malignant pleural effusions, mesotheliomas, Wilms tumors, trophoblastic neoplasms, hemangiopericytomas, Kaposi's sarcomas, myxoid carcinoma, round cell carcinoma, squamous cell carcinomas, esophageal squamous cell carcinomas, oral carcinomas, cancers of the adrenal cortex, or ACTH-producing tumors.

In some embodiments, provided herein is a method for treating a human who exhibits one or more symptoms associated with cancer (e.g., a hematologic malignancy). In some embodiments, the human is at an early stage of cancer. In other embodiments, the human is at an advanced stage of cancer.

In some embodiments, provided herein is a method for treating a human who is undergoing one or more standard therapies for treating cancer (e.g., a hematologic malignancy), such as chemotherapy, radiotherapy, immunotherapy, and/or surgery. Thus, in some foregoing embodiments, the combination of a BTK inhibitor and one or more inhibitor as described herein, may be administered before, during, or after administration of chemotherapy, radiotherapy, immunotherapy, and/or surgery. For example, the one or more inhibitor may be a JAK inhibitor, an ASK1 inhibitor, a BET inhibitor and a MMP9 inhibitor, as described herein. In some embodiments, Compound A1 may be used in combination with a JAK inhibitor such as Compound B1 and Compound B2. In other embodiments, Compound A1 may be used in combination with a BRD inhibitor such as (2-cyclopropyl-6-(3,5-dimethylisoxazol-4-yl)-1H-benzo[d]imidazol-4-yl)di(pyridin-2-yl)methanol.

In another aspect, provided is a method for sensitizing a human who is (i) refractory to at least one chemotherapy treatment, or (ii) in relapse after treatment with chemotherapy, or both (i) and (ii), wherein the method comprises administering a BTK inhibitor in combination with one or more inhibitor, as described herein, to the human. A human who is sensitized is a human who is responsive to the treatment involving administration of a BTK inhibitor in combination with one or more inhibitor, as described herein, or who has not developed resistance to such treatment. For example, the on or more inhibitor may be a JAK inhibitor, an ASK1 inhibitor, a BET inhibitor and/or a MMP9 inhibitor, as described herein.

For chronic lymphocytic leukemia the prior treatments a human may have received include regimens of:

fludarabine (Fludara®);

rituximab (Rituxan®);

rituximab (Rituxan®) combined with fludarabine (sometimes abbreviated as FR);

cyclophosphamide (Cytoxan®) combined with fludarabine; cyclophosphamide combined with rituximab and fludarabine (sometimes abbreviated as FCR);

cyclophosphamide combined with vincristine and prednisone (sometimes abbreviated as CVP);

cyclophosphamide combined with vincristine, prednisone, and rituximab;

combination of cyclophosphamide, doxorubicin, vincristine (Oncovin), and prednisone (sometimes referred to as CHOP);

Chlorambucil combined with prednisone, rituximab, obinutuzumab, or ofatumumab

pentostatin combined with cyclophosphamide and rituximab (sometimes abbreviated as PCR);

bendamustine (Treanda®) combined with rituximab ((sometimes abbreviated as BR);

alemtuzumab (Campath@);

fludarabine plus cyclophosphamide, bendamustine, or chlorambucil; and

fludarabine plus cyclophosphamide, bendamustine, or chlorambucil, combined with an anti-CD20 antibody, such as rituximab, ofatumumab, veltuzumab, lumiluximab or obinutuzumab.

In another aspect, provided herein is a methods for treating a human for a cancer, with comorbidity, wherein the treatment is also effective in treating the comorbidity. A “comorbidity” to cancer is a disease that occurs at the same time as the cancer.

The BTK inhibitor, Compound A1, or a pharmaceutically acceptable salt or hydrate thereof, may be combined with known agents and regimens useful in the treatment of allergic, autoimmune, and inflammatory disorders, as can the combinations herein of Compound A1 with one or more inhibitor as described herein. In addition, Compound A1 may be combined include tumor necrosis factor inhibitors (TNFi), such as infliximab (sold under the REMICADE® mark), etanercept (ENBREL®), certolizumab pegol (CIMZIA®), golimumab (SIMPONI®), adalimumab (HUMIRA®), and ozoalizumab.

### Therapeutically Effective Amounts

In some variations, a therapeutically effective amount refers to an amount that is sufficient to effect treatment, as defined below, when administered to a subject (e.g., a human) in need of such treatment. The therapeutically effective amount will vary depending upon the subject and disease condition being treated, the weight and age of the subject, the severity of the disease condition, the manner of administration and the like, which can readily be determined by one of ordinary skill in the art. For example, in one variation, a therapeutically effective amount of Compound A1, or a pharmaceutically acceptable salt or hydrate thereof, is an amount sufficient to modulate BTK expression, and thereby treat a human suffering an indication, or to ameliorate or alleviate the existing symptoms of the indication.

In another variation, the therapeutically effective amount of the BTK inhibitor, such as Compound A1, or a pharmaceutically acceptable salt or hydrate thereof, may be an amount sufficient to decrease a symptom of a disease or condition responsive to inhibition of BTK activity.

The compound, inhibitor, or therapeutic agent described herein may be administered using any suitable methods known in the art. For example, the compounds may be administered bucally, ophthalmically, orally, osmotically, parenterally (intramuscularly, intraperitoneally intrasternally, intravenously, subcutaneously), rectally, topically, transdermally, or vaginally. In certain embodiments, the Btk inhibitor is administered orally. In one embodiment, the Btk inhibitor is Compound A1 or hydrochloride salt thereof, which is administered orally, once a day, to a subject in need thereof at a dose of 20 mg, 40 mg, 80 mg, or 150 mg. In some embodiments, the Btk inhibitor is Compound A1 or hydrochloride salt thereof, which is administered orally, twice a day, to a subject at a dose of 20 mg, 40 mg, or 75 mg. In one variation, the therapeutically effective amount of the BTK inhibitor is a dose corresponding to 1 nmol to 10,000 nmol of the BTK inhibitor used in an apoptosis assay run with 10% serum which approximately relates to a blood plasma concentration of 500 nmol to 2500 nmol of the BTK inhibitor. In one variation, the therapeutically effective amount of the one or more inhibitor is a dose corresponding to 1 nmol to 200 nmol of the one or more inhibitor used in an apoptosis assay run with 10% serum. Specific examples include 3 nM, 5 nM, 10 nM, 20 nM and 30 nM concentrations when combined with one or more inhibitor described herein.

The therapeutically effective amount of the inhibitors described herein may also be determined based on data obtained from assays known in the art, including for example, an apoptosis assay. In one variation, the therapeutically effective amount of the BTK inhibitor in a human is a dose of from about 1 mg to about 200 mg. In another embodiment the BTK in a human is administered at a dose of from about 10 mg to about 200 mg. In another embodiment the BTK in a human is administered at a dose of from about 20 mg to about 160 mg. In other separate embodiments the BTK inhibitor is administered to a human at a dose of: a) from about 10 mg to about 100 mg, b) from about 50 mg to about 175 mg, c) from about 20 mg to about 150 mg, d) from about 75 mg to about 100 mg, and e) from about 100 mg to about 200 mg. Individual doses of the BTK inhibitor that may be administered to a human in need thereof include individual doses of 1 mg, 5 mg, 10 mg, 20 mg, 30 mg, 40 mg, 50 mg, 60 mg, 70 mg, 75 mg, 80 mg, 901 mg, 100 mg, 110 mg, 120 mg, 130 mg, 140 mg, 150 mg, 160 mg, 170 mg, 175 mg, and 200 mg. The doses of the BTK inhibitor may be administered as determined by a medical professional and may be administered once daily or may be delivered twice daily, three times daily, or four times daily.

In another variation, the BTK inhibitor, such as Compound A1, or a pharmaceutically acceptable salt or hydrate thereof, is administered to the human at a dose resulting in about 50%, about 55%, about 60%, about 65%, about 70%, about 75%, about 80%, about 90%, about 95%, or about 99% BTK target inhibition. In another variation, the one or more inhibitor, such as JAK inhibitor, ASK inhibitor, BRD inhibitor, and MMP9 inhibitor, is administered to the human at a dose resulting in about 50%, about 55%, about 60%, about 65%, about 70%, about 75%, about 80%, about 90%, about 95%, or about 99% target inhibition.

In some variations, the BTK inhibitor, such as Compound A1, or a pharmaceutically acceptable salt or hydrate thereof, is administered to the human at a dose between 40 mg and 1200 mg, between 40 mg and 800 mg, between 40 mg and 600 mg, between 40 mg and 40 mg, about 100 mg, about 100 mg, about 200 mg, about 300 mg, about 400 mg, about 500 mg, about 600 mg, about 700 mg, or about 800 mg. In some variations, the JAK inhibitor, such as Compound B1, Compound B2, Compound B3, or Compound B4, or a pharmaceutically acceptable salt thereof, is administered to the human at a dose between 20 to 600 mg, between 20 to 400 mg, between 20 to 200 mg, about 20 mg, about 50 mg, about 100 mg, about 200 mg, about 300 mg, about 400 mg, about 500 mg, about 600 mg, about 700 mg, or about 800 mg. In some embodiments, the JAK inhibitor is momelotinib (Compound B1) or a hydrochloride salt thereof is administered orally at a dose of 50 mg, 100 mg, 200 mg, or 400 mg. In certain embodiments, the JAK inhibitor is filgotinib (Compound B2) or a pharmaceutically salt thereof is administered orally at a dose of 30 mg. 50 mg, 75 mg, 100 mg, 150 mg, 200 mg, or 300 mg. In certain embodiments, the JAK inhibitor is administered once daily or twice daily.

The therapeutically effective amount of the BTK and one or more inhibitor described herein may be provided in a single dose or multiple doses to achieve the desired treatment endpoint. As used herein, “dose” refers to the total amount of an active ingredient to be taken each time by a human. The dose administered, for example for oral administration described above, may be administered once weekly, once daily (QD), twice daily (BID), three times daily, four times daily, or more than four times daily. In some embodiments, the BTK and/or the one or more inhibitor may be administered once daily. In some embodiments, the BTK and/or the one or more inhibitor may be administered twice daily. In some embodiments, the one or more inhibitor may be administered once weekly or with a frequency that can vary between daily, every other day, once every 5 days, daily for 1, 2, 3, 4, 5, 6 or 7 days and then weekly or with a regimen that can combine these different frequencies and doses to result in a final dose and regimen that is tolerated and efficacious.

In one variation, the therapeutically effective amount of the ASK1 inhibiting compound is a dose corresponding to 1 nmol to 200 nmol of the ASK1 inhibiting compound used in an apoptosis assay run with 10% serum. The Ask1 inhibiting compounds herein, including compounds of formula (I) and Compound C1, may be administered in a pharmaceutically effective amount. For oral administration, each dosage unit preferably contains from 1 mg to 500 mg of the ASK1 inhibiting compound. A more preferred dose is from 1 mg to 250 mg of the compound of the ASK1 inhibiting compound. Particularly preferred is a dose of the ASK1 inhibiting compound ranging from about 20 mg twice a day to about 50 mg twice a day. In some embodiments, the ASK inhibitor is Compound C2 which is administered orally at a dose of 2 mg, 6 mg, 10 mg, 18 mg, or 50 mg. In certain embodiments, the ASK inhibitor is administered once daily or twice daily. It will be understood, however, that the amount of the compound actually administered usually will be determined by a physician in light of the relevant circumstances including the condition to be treated, the chosen route of administration, co-administration compound if applicable, the age, weight, response of the individual patient, the severity of the patient's symptoms, and the like.

In some variations, the Btk inhibitor, such as Compound A1, or a pharmaceutically acceptable salt or hydrate thereof, is administered to the human at a dose between 40 mg and 1200 mg, between 40 mg and 800 mg, between 40 mg and 600 mg, between 40 mg and 40 mg, about 100 mg, about 100 mg, about 200 mg, about 300 mg, about 400 mg, about 500 mg, about 600 mg, about 700 mg, or about 800 mg. In some variations, the ASK1 inhibiting compound, such as Compound C1, Compound C2 or a compound of Formula I, or a pharmaceutically acceptable salt thereof, is administered to the human at a dose between 20 to 600 mg, between 20 to 400 mg, between 20 to 200 mg, about 20 mg, about 50 mg, about 100 mg, about 200 mg, about 300 mg, about 400 mg, about 500 mg, about 600 mg, about 700 mg, or about 800 mg.

In some variations, the BET inhibitor, such as a modulator of a bromodomain-containing protein, or a pharmaceutically acceptable salt thereof, as described herein, is administered to the human at a dose between 20 to 600 mg, between 20 to 400 mg, between 20 to 200 mg, about 20 mg, about 50 mg, about 100 mg, about 200 mg, about 300 mg, about 400 mg, about 500 mg, about 600 mg, about 700 mg, or about 800 mg.

In some variations, the MMP9 inhibitor, is administered to the human at a dose between 20 to 600 mg, between 20 to 400 mg, between 20 to 200 mg, about 20 mg, about 50 mg, about 100 mg, about 200 mg, about 300 mg, about 400 mg, about 500 mg, about 600 mg, about 700 mg, or about 800 mg.

In another embodiment, the MMP9 inhibitor, particularly including an anti-MMP9 antibody, is administered once every two weeks at a single dose of from about 600 mg to 1,000 mg. In another embodiment, the MMP9 inhibitor, particularly including an anti-MMP9 antibody, is administered once every two weeks at a single dose of from about 700 mg to about 900 mg. In another embodiment, the MMP9 inhibitor, particularly including an anti-MMP9 antibody, is administered once every two weeks at a single dose of from about 750 mg to about 850 mg. In another embodiment, the MMP9 inhibitor, particularly including an anti-MMP9 antibody, is administered once every two weeks at a single dose of about 800 mg. In another embodiment, the MMP9 inhibitor, particularly including an anti-MMP9 antibody, is administered once every three weeks at a single dose of from about 1,000 mg to 1,400 mg. In another embodiment, the MMP9 inhibitor, particularly including an anti-MMP9 antibody, is administered once every three weeks at a single dose of from about 1,100 mg to 1,300 mg. In another embodiment, the MMP9 inhibitor, particularly including an anti-MMP9 antibody, is administered once every three weeks at a single dose of about 1,200 mg. In one embodiment, the MMP9 inhibitor is an anti-MMP9 antibody having the amino acid sequence of SEQ ID Nos: 7 and 12, which is administered intravenously or subcutaneously at a dose of 150 mg, 300 mg, or 600 mg. In certain embodiment, the MMP9 inhibitor is administered once a week or once every two weeks.

The present disclosure contemplates pharmaceutical compositions for use in connection with such methods. Compositions can be suitable for administration locally or systemically by any suitable mute.

For example, when in vivo administration of an anti-MMP9 antibody is employed, normal dosage amounts can vary from about 10 ng/kg to up to 100 mg/kg of mammal body weight or more per day, preferably about 1 μg/kg/day to 50 mg/kg/day, optionally about 100 μg/kg/day to 20 mg/kg/day, 500 μg/kg/day to 10 mg/kg/day, or 1 mg/kg/day to 10 mg/kg/day, depending upon the route of administration.

The selected dosage regimen will depend upon a variety of factors including the activity of the MMP9 binding protein, the route of administration, the time of administration, the rate of excretion of the particular compound being employed, the duration of the treatment, other drugs, compounds and/or materials used in combination with the particular composition employed, the age, sex, weight, condition, general health and prior medical history of the patient being treated, and like factors well known in the medical arts.

A clinician having ordinary skill in the art can readily determine and prescribe the effective amount (ED50) of the pharmaceutical composition required. For example, the physician or veterinarian can start doses of the compounds of the invention employed in the pharmaceutical composition at levels lower than that required in order to achieve the desired therapeutic effect and gradually increase the dosage until the desired effect is achieved.

If needed, for cancer treatments, methods can further include surgical removal of the cancer and/or administration of an anti-cancer agent or treatment in addition to an MMP9 binding protein. Administration of such an anti-cancer agent or treatment can be concurrent with administration of the compositions disclosed herein.

### Administration

A BTK inhibitor, such a Compound A1, can be administered with one or more inhibitor using any suitable methods known in the art. For example, the one or more inhibitor to be combined with a BTK inhibitor may be a JAK inhibitor, such as Compound B1, Compound B2, Compound B3, Compound B4, Compound B5, Compound B6, Compound B7, Compound B8, Compound B9, Compound B10 or Compound B11. In some embodiments, the one or more inhibitor may be an ASK1 inhibitor, such as Compound C1, Compound C2 or a compound of Formula I. In other embodiments, the one or more inhibitor may be a modulator of a bromodomain-containing protein such as (2-cyclopropyl-6-(3,5-dimethylisoxazol-4-yl)-1H-benzo[d]imidazol-4-yl)di(pyridin-2-yl)methanol. In yet other embodiments, the one or more inhibitor may be a MMP9 inhibitor such as an anti-MMP9 antibody.

For example, the compounds may be administered bucally, ophthalmically, orally, osmotically, parenterally (intramuscularly, intraperitoneally intrasternally, intravenously, subcutaneously), rectally, topically, transdermally, or vaginally. Further, in certain variations, the BTK inhibitor described herein may be administered prior, after or concurrently with one or more inhibitor wherein the one or more inhibitor may be a JAK inhibitor, an ASK1 inhibitor, a BET inhibitor and a MMP9 inhibitor, as described herein.

In one aspect, the compounds described herein may be administered orally. Oral administration may be via, for example, capsule or enteric coated tablets. In making the pharmaceutical compositions that include at least one compound described herein, or a pharmaceutically acceptable salt thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound of the compounds described herein, or a pharmaceutically acceptable salt thereof, can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The compositions may, in some embodiments, be formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. In some embodiments, for oral administration, each dosage unit contains from about 10 mg to about 1000 mg of a compound described herein, for example from about 50 mg to about 500 mg, for example about 50 mg, about 75 mg, about 100 mg, about 150 mg, about 200 mg, about 250 mg, or about 300 mg. In other embodiments, for parenteral administration, each dosage unit contains from 0.1 to 700 mg of a compound a compound described herein. It will be understood, however, that the amount of the compound actually administered usually will be determined by a physician, in the light of the relevant circumstances, including the condition to be treated, the chosen route of administration, the actual compound administered and its relative activity, the age, weight, and response of the individual subject, and the severity of the subject's symptoms.

In certain embodiments, dosage levels may be from 0.1 mg to 100 mg per kilogram of body weight per day, for example from about 1 mg to about 50 mg per kilogram, for example from about 5 mg to about 30 mg per kilogram. Such dosage levels may, in certain instances, be useful in the treatment of the above-indicated conditions. In other embodiments, dosage levels may be from about 10 mg to about 2000 mg per subject per day. The amount of active ingredient that may be combined with the vehicle to produce a single dosage form will vary depending upon the host treated and the particular mode of administration. Dosage unit forms may contain from 1 mg to 500 mg of an active ingredient.

Frequency of dosage may also vary depending on the compound used and the particular disease or condition treated. In some embodiments, for example, for the treatment of an autoimmune and/or inflammatory disease, a dosage regimen of 4 times daily or less is used. In some embodiments, a dosage regimen of 1 or 2 times daily is used. It will be understood, however, that the specific dose level for any particular subject will depend upon a variety of factors including the activity of the specific compound employed, the age, body weight, general health, sex, diet, time of administration, route of administration, and rate of excretion, drug combination and the severity of the particular disease in the subject undergoing therapy.

For preparing solid compositions such as tablets, the principal active ingredient may be mixed with a pharmaceutical excipient to form a solid preformulation composition containing a homogeneous mixture of a compound of Formula (II), or a pharmaceutically acceptable salt thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The pharmaceutical compositions may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer

### Pharmaceutical Compositions

The BTK inhibitor and one or more inhibitor may be administered in the form of pharmaceutical compositions. For example, in some variations, the BTK inhibitor described herein may be present in a pharmaceutical composition comprising the BTK inhibitor, and at least one pharmaceutically acceptable vehicle. In some variations, the inhibitors described herein may be present in a pharmaceutical composition comprising the one or more inhibitor, and at least one pharmaceutically acceptable vehicle. For example, the one or more inhibitor may be a JAK inhibitor, an ASK1 inhibitor, a BET inhibitor and a MMP9 inhibitor. Pharmaceutically acceptable vehicles may include pharmaceutically acceptable carriers, adjuvants and/or excipients, and other ingredients can be deemed pharmaceutically acceptable insofar as they are compatible with other ingredients of the formulation and not deleterious to the recipient thereof.

This disclosure therefore provides pharmaceutical compositions that contain a BTK inhibitor and one or more inhibitor, wherein the one or more inhibitor may be a JAK inhibitor, an ASK1 inhibitor, a BET inhibitor and a MMP9 inhibitor as described herein, and one or more pharmaceutically acceptable vehicle, such as excipients, carriers, including inert solid diluents and fillers, diluents, including sterile aqueous solution and various organic solvents, permeation enhancers, solubilizers and adjuvants. The pharmaceutical compositions may be administered alone or in combination with other inhibitors. Such compositions are prepared in a manner well known in the pharmaceutical art (see, e.g., Remington's Pharmaceutical Sciences, Mace Publishing Co., Philadelphia, Pa. 17th Ed. (1985); and Modem Pharmaceutics, Marcel Dekker, Inc. 3rd Ed. (G. S. Banker & C. T. Rhodes, Eds.).

The pharmaceutical compositions may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

In some embodiments, the pharmaceutical compositions described herein are formulated in a unit dosage form. The term “unit dosage forms” refers to physically discrete units suitable as unitary dosages for human subjects, each unit containing a predetermined quantity of active material calculated to produce the desired therapeutic effect, in association with a suitable pharmaceutical excipient. In some variations, the pharmaceutical compositions described herein are in the form of a tablet, capsule, or ampoule.

In certain embodiments, the BTK inhibitor described herein, such as Compound A1, or a pharmaceutically acceptable salt or hydrate thereof, is formulated as a tablet. In some variations, such tablet may comprise a hydrochloride salt of Compound A1. Such tablet comprising Compound A1, for example, may be prepared by suitable methods known in the art, such as spray-drying and granulation (e.g., dry granulation).

### Articles of Manufacture and Kits

Compositions (including, for example, formulations and unit dosages) comprising a BTK inhibitor, as described herein, and compositions comprising one or more inhibitor, such as JAK inhibitors, ASK1 inhibitors, BET inhibitor and MMP9 inhibitors, as described herein, can be prepared and placed in an appropriate container, and labeled for treatment of an indicated condition. Accordingly, provided is also an article of manufacture, such as a container comprising a unit dosage form of a BTK inhibitor and a unit dosage form of a inhibitor, as described herein, and a label containing instructions for use of the compounds. In some embodiments, the article of manufacture is a container comprising (i) a unit dosage form of a BTK inhibitor, as described herein, and one or more pharmaceutically acceptable carriers, adjuvants or excipients; and (ii) a unit dosage form of a inhibitor, as described herein, and one or more pharmaceutically acceptable carriers, adjuvants or excipients. In one embodiment, the unit dosage form for both the BTK inhibitor and the one or more inhibitor is a tablet.

Kits also are contemplated. For example, a kit can comprise unit dosage forms of a BTK inhibitor, as described herein, and compositions comprising one or more inhibitor, as described herein, and a package insert containing instructions for use of the composition in treatment of a medical condition. For example, the one or more inhibitor may be a JAK inhibitor, an ASK1 inhibitor, a BET inhibitor and an MMP9 inhibitor. In some embodiments, the kits comprises (i) a unit dosage form of the BTK inhibitor, as described herein, and one or more pharmaceutically acceptable carriers, adjuvants or excipients; and (ii) a unit dosage form of a inhibitor, as described herein, and one or more pharmaceutically acceptable carriers, adjuvants or excipients. In one embodiment, the unit dosage form for both the BTK inhibitor and the inhibitor is a tablet.

The instructions for use in the kit may be for treating a cancer, including, for example, a hematologic malignancy, as further described herein. The instructions for use in the kit may be for treating a cancer, including, for example, a hematologic malignancy or an allergic, autoimmune, or inflammatory disorder, as further described herein.

### Other Therapeutic Agents

In the present disclosure, in some aspects, the combination therapies and methods described herein may be used or combined with an additional agents selected from the group of a chemotherapeutic agent, an anti-cancer agent, an anti-angiogenic agent, an anti-fibrotic agent, an immunotherapeutic agent, a therapeutic antibody, a radiotherapeutic agent, an anti-neoplastic agent, an anti-proliferation agent, or any combination thereof.

The combination therapies and methods described herein may be used or combined with an additional one or more of the following additional therapeutic agents: an adenosine A2B receptor (A2B) inhibitor, a BET-bromodomain 4 (BRD4) inhibitor, an isocitrate dehydrogenase 1 (IDH1) inhibitor, an IKK inhibitor, a protein kinase C (PKC) activator or inhibitor, a TPL2 inhibitor, a serine/threonine-protein kinase 1 (TBK) inhibitor, agents that activate or reactivate latent human immunodeficiency virus (HIV) such as panobinostat or romidepsin, an anti-CD20 antibody such as obinutuzumab, an anti-PD-1 antibody such as nivolimumab (BMS-936558, MDX1106, or MK-34775), and anti-PD-Li antibodies such as BMS-936559, MPDL3280A, MEDI4736, MSB0010718C, and MDX1105-01.

The combination therapies and methods disclosed herein and the additional one or more therapeutic agents (e.g. an A2B inhibitor, an apoptosis signal-regulating kinase (ASK) inhibitor, a BRD4 inhibitor, a discoidin domain receptor 1 (DDR1) inhibitor, a histone deacetylase (HDAC) inhibitor, an isocitrate dehydrogenase (IDH) inhibitor, a Janus kinase (JAK) inhibitor, a lysyl oxidase-like protein 2 (LOXL2) inhibitor, a matrix metalloprotease 9 (MMP9) inhibitor, a phosphatidylinositol 3-kinase (PI3K) inhibitor, a PKC activator or inhibitor, a spleen tyrosine kinase (SYK) inhibitor, a TPL2 inhibitor, or a TBK inhibitor) may be further used or combined with a chemotherapeutic agent, an anti-cancer agent, an anti-angiogenic agent, an anti-fibrotic agent, an immunotherapeutic agent, a therapeutic antibody, a radiotherapeutic agent, an anti-neoplastic agent, or any combination thereof.

It is understood that the combinations and methods herein may be used with standard therapies, including neoadjuvant chemotherapy, intraoperative radiotherapy (IORT), adjuvant chemotherapy (such as with 5U), adjuvant radiotherapy, adjuvant chemoradiotherapy, palliative radiotherapy, and palliative-intent procedures, which in regard to gastrointestinal conditions may include wide local excision, partial gastrectomy, total gastrectomy, simple laparotomy, gastrointestingal anastomosis, or bypass.

### Chemotherapeutic Agents

As used herein, the term “chemotherapeutic agent” or “chemotherapeutic” (or “chemotherapy” in the case of treatment with a chemotherapeutic agent) is meant to encompass any non-proteinaceous (i.e., non-peptidic) chemical compound useful in the treatment of cancer. Chemotherapeutic agents may be categorized by their mechanism of action into, for example, the following groups: anti-metabolites/anti-cancer agents such as pyrimidine analogs floxuridine, capecitabine, and cytarabine; purine analogs, folate antagonists, and related inhibitors; antiproliferative/antimitotic agents including natural products such as vinca alkaloid (vinblastine, vincristine) and microtubule such as taxane (paclitaxel, docetaxel), vinblastin, nocodazole, epothilones, vinorelbine (NAVELBINE), and epipodophyllotoxins (etoposide, teniposide); DNA damaging agents such as actinomycin, amsacrine, busulfan, carboplatin, chlorambucil, cisplatin, cyclophosphamide (CYTOXAN®), dactinomycin, daunorubicin, doxorubicin, epirubicin, iphosphamide, melphalan, merchlorethamine, mitomycin, mitoxantrone, nitrosourea, procarbazine, taxol, taxotere, teniposide, etoposide, and triethylenethiophosphoramide; antibiotics such as dactinomycin, daunorubicin, doxorubicin, idarubicin, anthracyclines, mitoxantrone, bleomycins, plicamycin (mithramycin), and mitomycin; enzymes such as L-asparaginase which systemically metabolizes L-asparagine and deprives cells which do not have the capacity to synthesize their own asparagine; antiplatelet agents; antiproliferative/antimitotic alkylating agents such as nitrogen mustards cyclophosphamide and analogs (melphalan, chlorambucil, hexamethylmelamine, and thiotepa), alkyl nitrosoureas (carmustine) and analogs, streptozocin, and triazenes (dacarbazine); antiproliferative/antimitotic antimetabolites such as folic acid analogs (methotrexate); platinum coordination complexes such as cisplatin, oxiloplatinim, and carboplatin), procarbazine, hydroxyurea, mitotane, and aminoglutethimide; hormones and hormone analogs such as estrogen, tamoxifen, goserelin, bicalutamide, and nilutamide, and aromatase inhibitors such as letrozole and anastrozole; anticoagulants such as heparin, synthetic heparin salts, and other inhibitors of thrombin; fibrinolytic agents such as tissue plasminogen activator, streptokinase, urokinase, aspirin, dipyridamole, ticlopidine, and clopidogrel; antimigratory agents; antisecretory agents such as breveldin; immunosuppressives such as tacrolimus, sirolimus, azathioprine, and mycophenolate; compounds (TNP-470, genistein) and growth factor inhibitors (vascular endothelial growth factor inhibitors and fibroblast growth factor inhibitors); angiotensin receptor blockers, nitric oxide donors; anti-sense oligonucleotides; antibodies such as trastuzumab and rituximab; cell cycle inhibitors and differentiation inducers such as tretinoin; inhibitors including topoisomerase inhibitors such as doxorubicin, daunorubicin, dactinomycin, eniposide, epirubicin, etoposide, idarubicin, irinotecan, mitoxantrone, topotecan, and irinotecan, and corticosteroids such as cortisone, dexamethasone, hydrocortisone, methylprednisolone, prednisone, and prednisolone; growth factor signal transduction kinase inhibitors; dysfunction inducers; toxins such as Cholera toxin, ricin, Pseudomonas exotoxin, Bordetella pertussis adenylate cyclase toxin, diphtheria toxin, and caspase activators; and chromatin.

Further examples of chemotherapeutic agents include: alkylating agents such as thiotepa and cyclophosphamide (CYTOXAN®); alkyl sulfonates such as busulfan, improsulfan, and piposulfan; aziridines such as benzodopa, carboquone, meturedopa, and uredopa; emylerumines and memylamelamines including alfretamine, triemylenemelamine, triethylenephosphoramide, triethylenethiophosphoramide, and trimemylolomelamine; acetogenins, especially bullatacin and bullatacinone; a camptothecin, including synthetic analog topotecan; bryostatin; callystatin; CC-1065, including its adozelesin, carzelesin, and bizelesin synthetic analogs; cryptophycins, particularly cryptophycin 1 and cryptophycin 8; dolastatin; duocarmycin, including the synthetic analogs KW-2189 and CBI-TMI; eleutherobin; pancratistatin; a sarcodictyin; spongistatin; nitrogen mustards such as chlorambucil, chlornaphazine, cyclophosphamide, estramustine, ifosfamide, mechlorethamine, mechlorethamine oxide hydrochloride, melphalan, novembichin, phenesterine, prednimustine, trofosfamide, and uracil mustard; nitrosoureas such as carmustine, chlorozotocin, foremustine, lomustine, nimustine, and ranimustine; antibiotics such as the enediyne antibiotics (e.g., calicheamicin, especially calicheamicin gammaII and calicheamicin phiIl), dynemicin including dynemicin A, bisphosphonates such as clodronate, an esperamicin, neocarzinostatin chromophore and related chromoprotein enediyne antibiotic chromomophores, aclacinomycins, actinomycin, authramycin, azaserine, bleomycins, cactinomycin, carabicin, carrinomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, and zombicin; anti-metabolites such as methotrexate and 5-fluorouracil (5-FU); folic acid analogs such as demopterin, methotrexate, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replinishers such as frolinic acid; trichothecenes, especially T-2 toxin, verracurin A, roridin A, and anguidine; taxoids such as paclitaxel (TAXOL®) and docetaxel (TAXOTERE®); platinum analogs such as cisplatin and carboplatin; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; pentostatin; phenamet; pirarubicin; losoxantrone; fluoropyrimidine; folinic acid; podophyllinic acid; 2-ethylhydrazide; procarbazine; polysaccharide-K (PSK); razoxane; rhizoxin; sizofiran; spirogermanium; tenuazonic acid; triaziquone; 2,2′,2″-tricUorotriemylamine; urethane; vindesine; dacarbazine; mannomustine; mitobronitol; mitolactol; pipobroman; gacytosine; arabinoside (“Ara-C”); cyclophosphamide; thiopeta; chlorambucil; gemcitabine (GEMZAR®); 6-thioguanine; mercaptopurine; methotrexate; vinblastine; platinum; etoposide (VP-16); ifosfamide; mitroxantrone; vancristine; vinorelbine (NAVELBINE®); novantrone; teniposide; edatrexate; daunomycin; aminopterin; xeoloda; ibandronate; CPT-11; topoisomerase inhibitor RFS 2000; difluoromethylornithine (DFMO); retinoids such as retinoic acid; capecitabine; FOLFIRI (fluorouracil, leucovorin, and irinotecan); and pharmaceutically acceptable salts, acids, or derivatives of any of the above.

### Anti-Hormonal Agents

Also included in the definition of“chemotherapeutic agent” are anti-hormonal agents such as anti-estrogens and selective estrogen receptor modulators (SERMs), inhibitors of the enzyme aromatase, anti-androgens, and pharmaceutically acceptable salts, acids or derivatives of any of the above that act to regulate or inhibit hormone action on tumors. Examples of anti-estrogens and SERMs include, for example, tamoxifen (including NOLVADEX™), raloxifene, droloxifene, 4-hydroxytamoxifen, trioxifene, keoxifene, LY117018, onapristone, and toremifene (FARESTON®). Inhibitors of the enzyme aromatase regulate estrogen production in the adrenal glands. Examples include 4(5)-imidazoles, aminoglutethimide, megestrol acetate (MEGACE®), exemestane, formestane, fadrozole, vorozole (RIVISOR®), letrozole (FEMARA®), and anastrozole (ARIMIDEX®). Examples of anti-androgens include flutamide, nilutamide, bicalutamide, leuprohde, and goserelin.

### Anti-Angiogenic Agents

Anti-angiogenic agents include, but are not limited to, retinoid acid and derivatives thereof, 2-methoxyestradiol, ANGIOSTATIN®, ENDOSTATIN®, suramin, squalamine, tissue inhibitor of metalloproteinase-1, tissue inhibitor of metalloproteinase-2, plasminogen activator inhibitor-1, plasminogen activator inhibitor-2, cartilage-derived inhibitor, paclitaxel (nab-paclitaxel), platelet factor 4, protamine sulphate (clupeine), sulphated chitin derivatives (prepared from queen crab shells), sulphated polysaccharide peptidoglycan complex (sp-pg), staurosporine, modulators of matrix metabolism including proline analogs ((1-azetidine-2-carboxylic acid (LACA)), cishydroxyproline, d,I-3,4-dehydroproline, thiaproline, α,α′-dipyridyl, beta-aminopropionitrile fumarate, 4-propyl-5-(4-pyridinyl)-2(3h)-oxazolone, methotrexate, mitoxantrone, heparin, interferons, 2 macroglobulin-serum, chicken inhibitor of metalloproteinase-3 (ChIMP-3), chymostatin, beta-cyclodextrin tetradecasulfate, eponemycin, fumagillin, gold sodium thiomalate, d-penicillamine, beta-1-anticollagenase-serum, alpha-2-antiplasmin, bisantrene, lobenzarit disodium, n-2-carboxyphenyl-4-chloroanthronilic acid disodium or “CCA”, thalidomide, angiostatic steroid, carboxy aminoimidazole, and metalloproteinase inhibitors such as BB-94. Other anti-angiogenesis agents include antibodies, preferably monoclonal antibodies against these angiogenic growth factors: beta-FGF, alpha-FGF, FGF-5, VEGF isoforms, VEGF-C, HGF/SF, and Ang-1/Ang-2.

### Anti-Fibrotic Agents

Anti-fibrotic agents include, but are not limited to, the compounds such as beta-aminoproprionitrile (BAPN), as well as the compounds disclosed in U.S. Pat. No. 4,965,288 relating to inhibitors of lysyl oxidase and their use in the treatment of diseases and conditions associated with the abnormal deposition of collagen and U.S. Pat. No. 4,997,854 relating to compounds which inhibit LOX for the treatment of various pathological fibrotic states, which are herein incorporated by reference. Further exemplary inhibitors are described in U.S. Pat. No. 4,943,593 relating to compounds such as 2-isobutyl-3-fluoro-, chloro-, or bromo-allylamine, U.S. Pat. No. 5,021,456, U.S. Pat. No. 5,059,714, U.S. Pat. No. 5,120,764, U.S. Pat. No. 5,182,297, U.S. Pat. No. 5,252,608 relating to 2-(1-naphthyloxymethyl)-3-fluoroallylamine, and US 2004-0248871, which are herein incorporated by reference.

Exemplary anti-fibrotic agents also include the primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives; semicarbazide and urea derivatives; aminonitriles such as BAPN or 2-nitroethylamine; unsaturated or saturated haloamines such as 2-bromo-ethylamine, 2-chloroethylamine, 2-trifluoroethylamine, 3-bromopropylamine, and p-halobenzylamines; and selenohomocysteine lactone. Other anti-fibrotic agents are copper chelating agents penetrating or not penetrating the cells. Exemplary compounds include indirect inhibitors which block the aldehyde derivatives originating from the oxidative deamination of the lysyl and hydroxylysyl residues by the lysyl oxidases. Examples include the thiolamines, particularly D-penicillamine, and its analogs such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphurate, 2-acetamidoethyl-2-acetamidoethanethiol sulphanate, and sodium-4-mercaptobutanesulphinate trihydrate.

### Immunotherapeutic Agents

The immunotherapeutic agents include and are not limited to therapeutic antibodies suitable for treating patients. Some examples of therapeutic antibodies include simtuzumab, abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nofetumomab, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, and 3F8. Rituximab can be used for treating indolent B-cell cancers, including marginal-zone lymphoma, WM, CLL and small lymphocytic lymphoma. A combination of Rituximab and chemotherapy agents is especially effective.

The exemplified therapeutic antibodies may be further labeled or combined with a radioisotope particle such as indium-111, yttrium-90, or iodine-131. In a certain embodiments, the additional therapeutic agent is a nitrogen mustard alkylating agent. Nonlimiting examples of nitrogen mustard alkylating agents include chlorambucil.

### Lymphoma or Leukemia Combination Therapy

Some chemotherapy agents are suitable for treating lymphoma or leukemia. These agents include aldesleukin, alvocidib, antincoplaston AS2-1, antineoplaston A10, anti-thymocyte globulin, amifostine trihydrate, aminocamptothecin, arsenic trioxide, beta alethine, Bcl-2 family protein inhibitor ABT-263, ABT-199, ABT-737, BMS-345541, bortezomib (VELCADE®), bryostatin 1, busulfan, carboplatin, campath-1H, CC-5103, carmustine, caspofungin acetate, clofarabine, cisplatin, cladribine, chlorambucil, curcumin, cyclosporine, cyclophosphamide, cytarabine, denileukin diftitox, dexamethasone, DT-PACE (dexamethasone, thalidomide, cisplatin, doxorubicin, cyclophosphamide, and etoposide), docetaxel, dolastatin 10, doxorubicin, doxorubicin hydrochloride, enzastaurin, epoetin alfa, etoposide, everolimus (RAD001), fenretinide, filgrastim, melphalan, mesna, flavopiridol, fludarabine, geldanamycin (17-AAG), ifosfamide, irinotecan hydrochloride, ixabepilone, lenalidomide (REVLIMID®, CC-5013), lymphokine-activated killer cells, melphalan, methotrexate, mitoxantrone hydrochloride, motexafin gadolinium, mycophenolate mofetil, nelarabine, oblimersen, obatoclax (GX15-070), oblimersen, octreotide acetate, omega-3 fatty acids, oxaliplatin, paclitaxel, PD0332991, PEGylated liposomal doxorubicin hydrochloride, pegfilgrastim, pentostatin, perifosine, prednisolone, prednisone, R-roscovitine (seliciclib, CYC202), recombinant interferon alfa, recombinant interleukin-12, recombinant interleukin-11, recombinant fit3 ligand, recombinant human thrombopoietin, rituximab, sargramostim, sildenafil citrate, simvastatin, sirolimus, styryl sulphones, tacrolimus, tanespimycin, temsirolimus (CCl-779), thalidomide, therapeutic allogeneic lymphocytes, thiotepa, tipifarnib, bortezomib (VELCADE®, PS-341), vincristine, vincristine sulfate, vinorelbine ditartrate, SAHA (suberanilohydroxamic acid, or suberoyl, anilide, and hydroxamic acid), FR (fludarabine and rituximab), CHOP (cyclophosphamide, doxorubicin, vincristine, and prednisone), CVP (cyclophosphamide, vincristine, and prednisone), FCM (fludarabine, cyclophosphamide, and mitoxantrone), FCR (fludarabine, cyclophosphamide, and rituximab), hyperCVAD (hyperfractionated cyclophosphamide, vincristine, doxorubicin, dexamethasone, methotrexate, and cytarabine), ICE (iphosphamide, carboplatin, and etoposide), MCP (mitoxantrone, chlorambucil, and prednisolone), R-CHOP (rituximab and CHOP), R-CVP (rituximab and CVP), R-FCM (rituximab and FCM), R-ICE (rituximab and ICE), and R-MCP (rituximab and MCP).

One modified approach is radioimmunotherapy, wherein a monoclonal antibody is combined with a radioisotope particle, such as indium-111, yttrium-90, and iodine-131. Examples of combination therapies include, but are not limited to, iodine-131 tositumomab (BEXXAR®), yttrium-90 ibritumomab tiuxetan (ZEVALIN®), and BEXXAR® with CHOP.

The abovementioned therapies can be supplemented or combined with stem cell transplantation or treatment. Therapeutic procedures include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme technique, low-LET cobalt-60 gamma ray therapy, bleomycin, conventional surgery, radiation therapy, and nonmyeloablative allogeneic hematopoietic stem cell transplantation.

### Non-Hodgkin's Lymphomas Combination Therapy

Treatment of non-Hodgkin's lymphomas (NHL), especially those of B cell origin, includes using monoclonal antibodies, standard chemotherapy approaches (e.g., CHOP, CVP, FCM, MCP, and the like), radioimmunotherapy, and combinations thereof, especially integration of an antibody therapy with chemotherapy. Examples of unconjugated monoclonal antibodies for the treatment of NHL/B-cell cancers include rituximab, alemtuzumab, human or humanized anti-CD20 antibodies, lumiliximab, anti-TNF-related apoptosis-inducing ligand (anti-TRAIL), bevacizumab, galiximab, epratuzumab, SGN-40, and anti-CD74. Examples of experimental antibody agents used in treatment of NHL/B-cell cancers include ofatumumab, ha20, PRO131921, alemtuzumab, galiximab, SGN-40, CHIR-12.12, epratuzumab, lumiliximab, apolizumab, milatuzumab, and bevacizumab. Examples of standard regimens of chemotherapy for NHL/B-cell cancers include CHOP, FCM, CVP, MCP, R-CHOP, R-FCM, R-CVP, and R-MCP. Examples of radioimmunotherapy for NHL/B-cell cancers include yttrium-90 ibritumomab tiuxetan (ZEVALIN®) and iodine-131 tositumomab (BEXXAR®).

### Mantle Cell Lymphoma Combination Therapy

Therapeutic treatments for mantle cell lymphoma (MCL) include combination chemotherapies such as CHOP, hyperCVAD, and FCM. These regimens can also be supplemented with the monoclonal antibody rituximab to form combination therapies R-CHOP, hyperCVAD-R, and R-FCM. Any of the abovementioned therapies may be combined with stem cell transplantation or ICE in order to treat MCL.

An alternative approach to treating MCL is immunotherapy. One immunotherapy uses monoclonal antibodies like rituximab. Another uses cancer vaccines, such as GTOP-99, which are based on the genetic makeup of an individual patient's tumor.

A modified approach to treat MCL is radioimmunotherapy, wherein a monoclonal antibody is combined with a radioisotope particle, such as iodine-131 tositumomab (BEXXAR®) and yttrium-90 ibritumomab tiuxetan (ZEVALIN®). In another example, BEXXAR® is used in sequential treatment with CHOP.

Other approaches to treating MCL include autologous stem cell transplantation coupled with high-dose chemotherapy, administering proteasome inhibitors such as bortezomib (VELCADE® or PS-341), or administering antiangiogenesis agents such as thalidomide, especially in combination with rituximab.

Another treatment approach is administering drugs that lead to the degradation of Bcl-2 protein and increase cancer cell sensitivity to chemotherapy, such as oblimersen, in combination with other chemotherapeutic agents.

A further treatment approach includes administering mTOR inhibitors, which can lead to inhibition of cell growth and even cell death. Non-limiting examples are temsirolimus (TORISEL®, CCI-779) and temsirolimus in combination with RITUXAN®, VELCADE®, or other chemotherapeutic agents.

Other recent therapies for MCL have been disclosed. Such examples include flavopiridol, PD0332991, R-roscovitine (selicicilib, CYC202), styryl sulphones, obatoclax (GX15-070), TRAIL, Anti-TRAIL death receptors DR4 and DR5 antibodies, temsirolimus (TORISEL®, CCI-779), everolimus (RAD001), BMS-345541, curcumin, SAHA, thalidomide, lenalidomide (REVLIMID®, CC-5013), and geldanamycin (17-AAG).

### Waldenstrom's Macroglobulinemia Combination Therapy

Therapeutic agents used to treat Waldenstrom's Macroglobulinemia (WM) include perifosine, bortezomib (VELCADE®), rituximab, sildenafil citrate (VIAGRA®), CC-5103, thalidomide, epratuzumab (hLL2-anti-CD22 humanized antibody), simvastatin, enzastaurin, campath-1H, dexamethasone, DT-PACE, oblimersen, antineoplaston A10, antineoplaston AS2-1, alemtuzumab, beta alethine, cyclophosphamide, doxorubicin hydrochloride, prednisone, vincristine sulfate, fludarabine, filgrastim, melphalan, recombinant interferon alfa, carmustine, cisplatin, cyclophosphamide, cytarabine, etoposide, melphalan, dolastatin 10, indium-111 monoclonal antibody MN-14, yttrium-90 humanized epratuzumab, anti-thymocyte globulin, busulfan, cyclosporine, methotrexate, mycophenolate mofetil, therapeutic allogeneic lymphocytes, yttrium-90 ibritumomab tiuxetan, sirolimus, tacrolimus, carboplatin, thiotepa, paclitaxel, aldesleukin, docetaxel, ifosfamide, mesna, recombinant interleukin-11, recombinant interleukin-12, Bcl-2 family protein inhibitor ABT-263, denileukin diftitox, tanespimycin, everolimus, pegfilgrastim, vorinostat, alvocidib, recombinant flt3 ligand, recombinant human thrombopoietin, lymphokine-activated killer cells, amifostine trihydrate, aminocamptothecin, irinotecan hydrochloride, caspofungin acetate, clofarabine, epoetin alfa, nelarabine, pentostatin, sargramostim, vinorelbine ditartrate, WT-1 analog peptide vaccine, WT1 126-134 peptide vaccine, fenretinide, ixabepilone, oxaliplatin, monoclonal antibody CD19, monoclonal antibody CD20, omega-3 fatty acids, mitoxantrone hydrochloride, octreotide acetate, tositumomab, iodine-131 tositumomab, motexafin gadolinium, arsenic trioxide, tipifarnib, autologous human tumor-derived HSPPC-96, veltuzumab, bryostatin 1, PEGylated liposomal doxorubicin hydrochloride, and any combination thereof.

Examples of therapeutic procedures used to treat WM include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme techniques, low-LET cobalt-60 gamma ray therapy, bleomycin, conventional surgery, radiation therapy, and nonmyeloablative allogeneic hematopoietic stem cell transplantation.

### Diffuse Large B-cell Lymphoma Combination Therapy

Therapeutic agents used to treat diffuse large B-cell lymphoma (DLBCL) include cyclophosphamide, doxorubicin, vincristine, prednisone, anti-CD20 monoclonal antibodies, etoposide, bleomycin, many of the agents listed for WM, and any combination thereof, such as ICE and R-ICE.

### Chronic Lymphocytic Leukemia Combination Therapy

Examples of therapeutic agents used to treat chronic lymphocytic leukemia (CLL) include chlorambucil, cyclophosphamide, fludarabine, pentostatin, cladribine, doxorubicin, vincristine, prednisone, prednisolone, alemtuzumab, many of the agents listed for WM, and combination chemotherapy and chemoimmunotherapy, including the following common combination regimens: CVP, R-CVP, ICE, R-ICE, FCR, and FR.

### Myelofibrosis Combination Therapy

Myelofibrosis inhibiting agents include, but are not limited to, hedgehog inhibitors, histone deacetylase (HDAC) inhibitors, and tyrosine kinase inhibitors. A non-limiting example of hedgehog inhibitors is saridegib. Examples of HDAC inhibitors include, but are not limited to, pracinostat and panobinostat. A non-limiting example of a tyrosine kinase inhibitor is lestaurtinib.

### Kinase Inhibitors

In one embodiment, the compound described herein may be used or combined with one or more additional therapeutic agents. The one or more therapeutic agents include, but are not limited to, an inhibitor of Abl, activated CDC kinase (ACK), adenosine A2B receptor (A2B), apoptosis signal-regulating kinase (ASK), Auroa kinase, BET-bromodomain (BRD) such as BRD4, c-Kit, c-Met, CDK-activating kinase (CAK), calmodulin-dependent protein kinase (CaMK), cyclin-dependent kinase (CDK), casein kinase (CK), discoidin domain receptor (DDR), epidermal growth factor receptors (EGFR), focal adhesion kinase (FAK), Flt-3, FYN, glycogen synthase kinase (GSK), HCK, histone deacetylase (HDAC), IKK such as IKKβε, isocitrate dehydrogenase (IDH) such as IDH1, Janus kinase (JAK), KDR, lymphocyte-specific protein tyrosine kinase (LCK), lysyl oxidase protein, lysyl oxidase-like protein (LOXL), LYN, matrix metalloprotease (MMP), MEK, mitogen-activated protein kinase (MAPK), NEK9, NPM-ALK, p38 kinase, platelet-derived growth factor (PDGF), phosphorylase kinase (PK), polo-like kinase (PLK), phosphatidylinositol 3-kinase (PI3K), protein kinase (PK) such as protein kinase A, B, and/or C, PYK, spleen tyrosine kinase (SYK), serine/threonine kinase TPL2, serine/threonine kinase STK, signal transduction and transcription (STAT), SRC, serine/threonine-protein kinase (TBK) such as TBKI, TIE, tyrosine kinase (TK), vascular endothelial growth factor receptor (VEGFR), YES, or any combination thereof.

### Apoptosis Signal-Regulating Kinase (ASK) Inhibitors

ASK inhibitors include ASK1 inhibitors. Examples of ASK1 inhibitors include, but are not limited to, those described in WO 2011/008709 (Gilead Sciences) and WO 2013/112741 (Gilead Sciences).

### Discoidin Domain Receptor (DDR) Inhibitors

DDR inhibitors include inhibitors of DDR1 and/or DDR2. Examples of DDR inhibitors include, but are not limited to, those disclosed in WO 2014/047624 (Gilead Sciences), US 2009-0142345 (Takeda Pharmaceutical), US 2011-0287011 (Oncomed Pharmaceuticals), WO 2013/027802 (Chugai Pharmaceutical), and WO 2013/034933 (Imperial Innovations).

### Histone Deacetylase (HDAC) Inhibitors

Examples of HDAC inhibitors include, but are not limited to, pracinostat and panobinostat.

### Janus Kinase (JAK) Inhibitors

JAK inhibitors inhibit JAK1, JAK2, and/or JAK3. Examples of JAK inhibitors include, but are not limited to, Compound A, ruxolitinib, fedratinib, tofacitinib, baricitinib, lestaurtinib, pacritinib, XL019, AZD1480, INCB039110, LY2784544, BMS911543, and NS018.

### Lysyl Oxidase-Like Protein (LOXL) Inhibitors

LOXL inhibitors include inhibitors of LOXL1, LOXL2, LOXL3, LOXL4, and/or LOXL5. Examples of LOXL inhibitors include, but are not limited to, the antibodies described in WO 2009/017833 (Arresto Biosciences). Examples of LOXL2 inhibitors include, but are not limited to, the antibodies described in WO 2009/017833 (Arresto Biosciences), WO 2009/035791 (Arresto Biosciences), and WO 2011/097513 (Gilead Biologics).

### Matrix Metalloprotease (MMP) Inhibitors

MMP inhibitors include inhibitors of MMP1 through 10. Examples of MMP9 inhibitors include, but are not limited to, marimastat (BB-2516), cipemastat (Ro 32-3555), and those described in WO 2012/027721 (Gilead Biologics).

### Phosphatidylinositol 3-Kinase (PI3K) Inhibitors

PI3K inhibitors include inhibitors of PI3Kγ, PI3Kδ, PI3Kβ, PI3Kα, and/or pan-PI3K. Examples of PI3K inhibitors include, but are not limited to, wortmannin, BKM120, CH5132799, XL756, and GDC-0980. Examples of PI3Kγ inhibitors include, but are not limited to, ZSTK474, AS252424, LY294002, and TG100115. Examples of PI3KS inhibitors include, but are not limited to, PI3K II, TGR-1202, AMG-319, GSK2269557, X-339, X-414, RP5090, KAR4141, XL499, OXY111A, IPI-145, IPI-443, and the compounds described in WO 2005/113556 (ICOS), WO 2013/052699 (Gilead Calistoga), WO 2013/116562 (Gilead Calistoga), WO 2014/100765 (Gilead Calistoga), WO 2014/100767 (Gilead Calistoga), and WO 2014/201409 (Gilead Sciences). Examples of PI3Kβ inhibitors include, but are not limited to, GSK2636771, BAY 10824391, and TGX221. Examples of PI3Kα inhibitors include, but are not limited to, buparlisib, BAY 80-6946, BYL719, PX-866, RG7604, MLN1117, WX-037, AEZA-129, and PA799. Examples of pan-PI3K inhibitors include, but are not limited to, LY294002, BEZ235, XL147 (SAR245408), and GDC-0941.

### Spleen Tyrosine Kinase (SYK) Inhibitors

Examples of SYK inhibitors include, but are not limited to, tamatinib (R406), fostamatinib (R788), PRT062607, BAY-61-3606, NVP-QAB 205 AA, R112, R343, and those described in U.S. Pat. No. 8,450,321 (Gilead Connecticut).

### Tyrosine-Kinase Inhibitors (TKIs)

TKIs may target epidermal growth factor receptors (EGFRs) and receptors for fibroblast growth factor (FGF), platelet-derived growth factor (PDGF), and vascular endothelial growth factor (VEGF). Examples of TKIs that target EGFR include, but are not limited to, gefitinib and erlotinib. Sunitinib is a non-limiting example of a TKI that targets receptors for FGF, PDGF, and VEGF.

Combinations of pharmaceutically effective amounts of the BTK inhibitor and an ASK1 inhibitor as described herein may also be used to treat an allergic disorders, autoimmune diseases and inflammatory diseases in a human, the method comprising administering to the human in need thereof a pharmaceutically effective amount of the BTK inhibitor, or a pharmaceutically acceptable salt or hydrate thereof, and a pharmaceutically effective amount of an ASK1 inhibitor, or a pharmaceutically acceptable salt or hydrate thereof. Particularly, the combinations taught herein may be used for the treatment of allergic disorders, autoimmune diseases and inflammatory diseases such as: systemic lupus erythematosus (SLE), rheumatoid arthritis, multiple vasculitides, idiopathic thrombocytopenic purpura (ITP), myasthenia gravis, allergic rhinitis, chronic obstructive pulmonary disease (COPD), adult respiratory distress syndrome (ARDs) and asthma.

### EXAMPLES

The following examples are provided to further aid in understanding the embodiments disclosed in the application, and presuppose an understanding of conventional methods well known to those persons having ordinary skill in the art to which the examples pertain. The particular materials and conditions described hereunder are intended to exemplify particular aspects of embodiments disclosed herein and should not be construed to limit the reasonable scope thereof. It is understood that the conditions (such as the reagent concentration or the incubation temperature) of the assay or study may be varied and the results of the assay or study may vary. In some instances, the value may vary within a range of one to three-fold.

### Example 1

This study evaluated the potential effects of BTK inhibitor in combination with JAK inhibitor in treating arthritis. Lewis rats were injected intradermally/subcutaneously (ID/SC) with porcine type II collagen to induce arthritis. Arthritic rats were treated with vehicle (20% Cremophor EL/10% EtOH/70% saline), Compound A1 (a BTK inhibitor), Compound B4 (a JAK inhibitor), Compound A1 and Compound B4, or Dex (dexamethasone). Compound A1 was administered orally either twice daily at 3, 10, or 20 mg/kg or once daily at 20 mg/kg; Compound B4 was administered orally daily at 2.5 mg/kg; dex was administered daily at 0.075 mg/kg, initiated on day 17. The study was terminated at day 34. Efficacy evaluation was based on body weights, daily ankle caliper measurements, ankle diameter expressed as area under the curve (AUC), terminal hind paw weights, and histopathologic evaluation of right ankles.

This model may reflect certain clinical and histopathologic parameters, such as inflammation, cartilage destruction, and bone resorption that occur in established type II collagen arthritis in female Lewis rats. As the treatment was initiated at the peak of established disease and continued into the chronic phase; the results obtained may be used in evaluating chronic, highly destructive macrophage-mediated phase of this model.

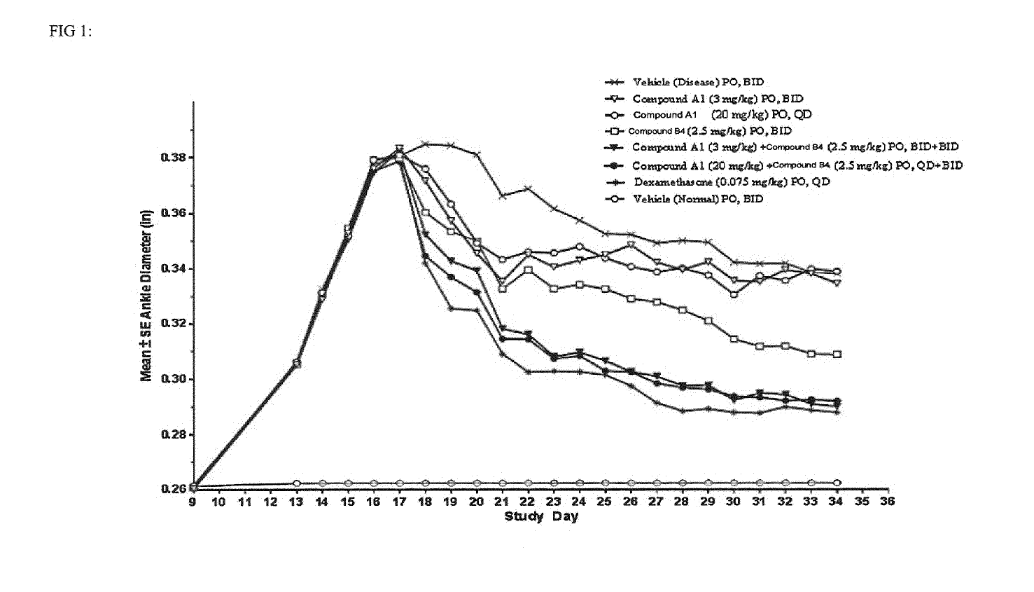

Ankle diameters were measured and compared for potential treatment effects. FIG. 1 depicts the measurements taken on Day 9, 13-34 for ankle diameter (in.) (mean±standard error) for the following groups: control (normal and disease), Compound A1 (20 mg/kg, daily), Compound A1 (3 mg/kg, twice daily), Compound B4 (2.5 mg/kg, twice daily), Compound A1 (20 mg/kg, daily) with Compound B4 (2.5 mg/kg, twice daily), Compound A1 (3 mg/kg, twice daily) with Compound B4 (2.5 mg/kg, twice daily) and Dex (0.075 mg/kg daily). In addition, the AUC total sum (day 17-34) (mean f standard error) was measured. The AUC total sum for the control (normal) was 4.5±0.008; for control (disease), 6.1±0.058; for Compound A1 (20 mg/kg, daily), 5.9±0.096; for Compound A1 (20 mg/kg, twice daily), 5.8±0.124; for Compound A1 (10 mg/kg, twice daily), 5.9±0.102; for Compound A1 (3 mg/kg, twice daily), 5.9±0.079; for Compound B4 (2.5 mg/kg, twice daily), 5.6±0.083; for Compound A1 (20 mg/kg, daily) with Compound B4 (2.5 mg/kg, twice daily), 5.3±0.063; for Compound A1 (10 mg/kg, twice daily) with Compound B4 (2.5 mg/kg, twice daily), 5.3±0.093; for Compound A1 (3 mg/kg, twice daily) with Compound B4 (2.5 mg/kg, twice daily), 5.3±0.082; and for Dex (0.075 mg/kg daily), 5.2±0.069.

Also, the percent inhibition of the AUC total sum (day 17-34) was determined. The percent inhibition was 100% for the control (normal); 0% for the control (disease); 13% for Compound A1 (20 mg/kg, daily); 15% for Compound A1 (20 mg/kg, twice daily); 9% for Compound A1 (10 mg/kg, twice daily); 13% for Compound A1 (3 mg/kg, twice daily); 28% for Compound B4 (2.5 mg/kg, twice daily); 50% for Compound A (20 mg/kg, daily) with Compound B4 (2.5 mg/kg, twice daily); 49% for Compound A1 (10 mg/kg, twice daily) with Compound B4 (2.5 mg/kg, twice daily); 48% for Compound A1 (3 mg/kg, twice daily) with Compound B4 (2.5 mg/kg, twice daily); and 56% for Dex (0.075 mg/kg).

The following score systems were used to evaluate ankle inflammation, ankle pannus, ankle cartilage damage, ankle bone resorption, and periosteal new bone formation, which may represent treatment effects to ankle histology. The sum of the summed ankle histology scores for day 34, are provided herein.

Ankle inflammation scores as used herein have the following meaning: 0=normal; 0.5=minimal focus inflammation; 1=minimal infiltration of inflammatory cells in synovium/periarticular tissue; 2=mild infiltration; 3-moderate infiltration with moderate edema; 4=marked infiltration with marked edema; 5=severe infiltration with severe edema. Ankle pannus scores as used herein have the following meaning: 0=normal; 0.5=minimal infiltration of pannus in cartilage and subchondral bone, affects only marginal zones and affects only a few joints; 1=minimal infiltration of pannus in cartilage and subchondral bone, primarily affects marginal zones; 2=mild infiltration (<25% of tibia or tarsals at marginal zones); 3=moderate infiltration (26%-50% of tibia or small tarsals affected at marginal zones); 4=marked infiltration (51%-75% of tibia or tarsals affected at marginal zones); 5=severe infiltration (>75% of tibia or tarsals affected at marginal zones, severe distortion of overall architecture).

Ankle cartilage damage scores as used herein have the following meaning: 0=normal; 0.5=minimal decrease in T blue staining, affects only marginal zones and affects only a few joints; 1=minimal to mild loss of toluidine blue staining with no obvious chondrocyte loss or collagen disruption; 2=mild loss of toluidine blue staining with focal mild (superficial) chondrocyte loss and/or collagen disruption; 3=moderate loss of toluidine blue staining with multifocal moderate (depth to middle zone) chondrocyte loss and/or collagen disruption, smaller tarsals affected to 50% to 75% depth with rare areas of full thickness loss; 4=marked loss of toluidine blue staining with multifocal marked (depth to deep zone) chondrocyte loss and/or collagen disruption, 1 or 2 small tarsals surfaces have full thickness loss of cartilage; 5=severe diffuse loss of toluidine blue staining with multifocal severe (depth to tide mark) chondrocyte loss and/or collagen disruption affecting more than 2 cartilage surfaces.

Ankle bone resorption scores as used herein have the following meaning: 0=normal; 0.5=minimal resorption affects only marginal zones and affects only a few joints; 1=small areas of resorption, not readily apparent on low magnification, rare osteoclasts; 2=more numerous areas of resorption, not readily apparent on low magnification, osteoclasts more numerous, <25% of tibia or tarsals at marginal zones resorbed; 3=obvious resorption of medullary trabecular and cortical bone without full thickness defects in cortex, loss of some medullary trabeculae, lesion apparent on low magnification, osteoclasts more numerous, 25% to 50% of tibia or tarsals affected at marginal zones; 4=full thickness defects in cortical bone, often with distortion of profile of remaining cortical surface, marked loss of medullary bone, numerous osteoclasts, 51% to 75% of tibia or tarsals affected at marginal zones; 5=full thickness defects in cortical bone, often with distortion of profile of remaining cortical surface, marked loss of medullary bone, numerous osteoclasts, >75% of tibia or tarsals affected at marginal zones, severe distortion of overall architecture.

Periosteal new bone formation scores as used herein have the following meaning: 0=normal, no periosteal proliferation; 0.5=minimal focal or multifocal proliferation, measures less than 127 um width (1-2 units at 16×) at any location; 1=minimal multifocal proliferation, width at any location measures 127-252 um (3-4 units at 16×); 2=mild multifocal on tarsals, diffuse in some locations, width at any location 253-441 um (5-7 units at 16×); 3=moderate multifocal on tarsals, diffuse in most other locations, width at any location measures 442-630 um (8-10 units at 16×); 4=marked multifocal on tarsals, diffuse at most other locations, width at any location measures 630-819 um (11-13 units at 16×); 5=severe, multifocal on tarsals, diffuse at most other locations, width at any location measures >819 um (>13 units at 16×).

The summed ankle histopathology (mean±standard error) was measured by histopathology scores. The sum of inflammation, pannus, cartilage damage, bone resorption and periosteal new bone formation was calculated for each ankle, with a maximum value of 25. The summed ankle histopathology for the control (normal) was 0±0.0; for control (disease), 25±0.0; for Compound A1 (20 mg/kg, daily), 21±0.7; for Compound A1 (20 mg/kg, twice daily), 21±0.6; for Compound A1 (10 mg/kg, twice daily), 21±1.4; for Compound A1 (3 mg/kg, twice daily), 23±0.6; for Compound B4 (2.5 mg/kg, twice daily), 19±1.4; for Compound A1 (20 mg/kg, daily) with Compound B4 (2.5 mg/kg, twice daily), 10±1.2; for Compound A1 (10 mg/kg, twice daily) with Compound B4 (2.5 mg/kg, twice daily), 11±1.6; for Compound A1 (3 mg/kg, twice daily) with Compound B4 (2.5 mg/kg, twice daily), 10±2.0; and for Dex (0.075 mg/kg daily), 12±1.0.

For the summed ankle histopathology, the percent inhibition was determined. The percent inhibition was 100% for the control (normal); 0% for the control (disease); 15% for Compound A1 (20 mg/kg, daily); 18% for Compound A1 (20 mg/kg, twice daily); 16% for Compound A1 (10 mg/kg, twice daily); 10% for Compound A1 (3 mg/kg, twice daily); 23% for Compound B4 (2.5 mg/kg, twice daily); 60% for Compound A1 (20 mg/kg, daily) with Compound B4 (2.5 mg/kg, twice daily); 57% for Compound A1 (10 mg/kg, twice daily) with Compound B4 (2.5 mg/kg, twice daily); 60% for Compound A1 (3 mg/kg, twice daily) with Compound B4 (2.5 mg/kg, twice daily); and 51% for Dex (0.075 mg/kg).

In addition, the ED-1 immunopositive osteoclast count (mean f standard error) was measured. The ED-1 immunopositive osteoclast count for the control (normal) was 1±0.2; for the control (disease), 19±1.0; for Compound A1 (20 mg/kg, daily), 9±1.5; for Compound A1 (20 mg/kg, twice daily), 4±0.7; for Compound A1 (10 mg/kg, twice daily), 8±1.9; for Compound A1 (3 mg/kg, twice daily), 7±1.3; for Compound B4 (2.5 mg/kg, twice daily), 16±1.7 for Compound A1 (20 mg/kg, daily) with Compound B4 (2.5 mg/kg, twice daily), 4±0.4; Compound A1 (10 mg/kg, twice daily) with Compound B4 (2.5 mg/kg, twice daily), 3±0.4; Compound A1 (3 mg/kg, twice daily) with Compound B4 (2.5 mg/kg, twice daily), 3±0.4; and for Dex (0.075 mg/kg), 4±1.4. For the ED-1 immunopositive osteoclast count, the percent inhibition was also measured. The percent inhibition for the control (normal) was 100%; for the control (disease), 0%; for Compound A1 (20 mg/kg, daily), 56%; for Compound A1 (20 mg/kg, twice daily), 83%; for Compound A1 (10 mg/kg, twice daily), 62%; for Compound A1 (3 mg/kg, twice daily), 65%; for Compound B4 (2.5 mg/kg, twice daily), 14%; for Compound A1 (20 mg/kg, daily) with Compound B4 (2.5 mg/kg, twice daily), 85%; Compound A1 (10 mg/kg, twice daily) with Compound B4 (2.5 mg/kg, twice daily), 91%; Compound A1 (3 mg/kg, twice daily) with Compound B4 (2.5 mg/kg, twice daily), 90%; and for Dex (0.075 mg/kg), 85%.

### Example 2

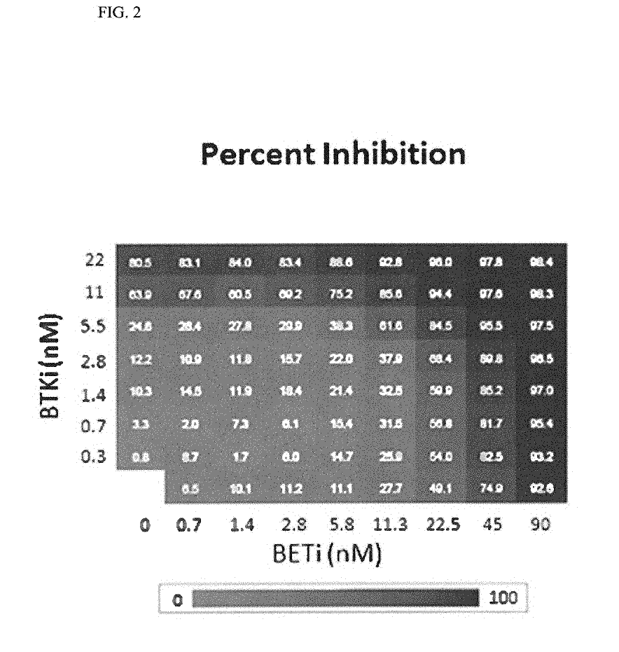

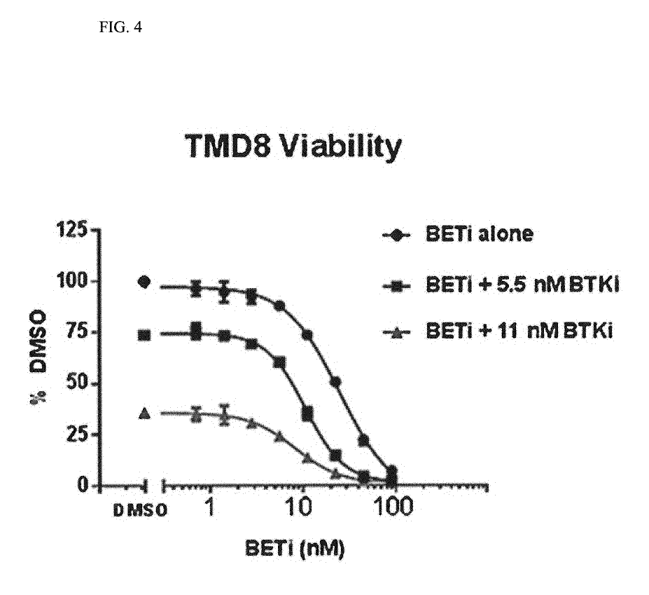

Material and Methods: The effect of the combination of a BET inhibitor, (2-cyclopropyl-6-(3,5-dimethylisoxazol-4-yl)-1H-benzo[d]imidazol-4-yl)di(pyridin-2-yl)methanol (Compound D), and BTK inhibitor (Compound A1) on growth inhibition of the human activated B cell (ABC) subtype DLBCL cell line, TMD8, was evaluated in vitro. TMD8 cells were dosed with a matrix of Compound D (0-90 nM) and compound A1 (0-22 nM) and treated for four days, after which cell viability was measured by a CellTiter Glo assay. A representative heatmap of this dose matrix for cell growth inhibition is shown in FIG. 2 (0% to 100% growth inhibition). Both compounds reduced cell growth over the dose range; synergy was observed at concentrations of 5.8-90 nM of Compound D and 0.3-22 nM of Compound A1 (FIG. 3). Synergy was defined as the excess over the predicted additive interaction between the compounds using Bliss analysis. The dose response curve for growth inhibition of Compound D alone or in the presence of 5.5 nM or 11 nM of Compound A1 is shown in FIG. 4. The average IC₅₀ values (concentration that causes half maximal inhibition of cell growth) for Compound D were decreased from 25 nM to 11 nM and 8 nM by the presence of 5.5 nM and 11 nM of Compound A1, respectively, and is consistent with a synergistic interaction.

### Cell Viability Assay:

Cells were plated at a density of 4,000 cells per well in 384-well (Grenier 781086) tissue culture black well plates already spotted with compounds by a Labcyte Echo liquid handler. Cells were treated with an 8-point 2-fold dilution series of (Compound D starting at 90 nM (final DMSO concentration of 0.14%). Cells treated with DMSO alone were used as a positive control for 100% cell growth. Cells were treated with Compound D alone or in the presence of a dose range of Compound A1 (6-point 2-fold dilution series ranging from 0.3-22 nM) for each dose of Compound D. Cells were incubated at 37° C. for 96 hours and viability was measured using CellTiterGlo reagent as per the vendor's protocol. Curves were plotted in prism and IC₅₀ values were calculated with a 4-parameter variable hillslope non-linear fit. The predicted response under Bliss additivity for any combination of drugs at a given concentration pair was determined by Ra+Rb−Ra*Rb, where Ra and Rb are the responses of Compounds D and A1 (i.e., cell growth inhibition). The total Bliss score was determined by summing the differences between the observed values and the predicted additive value at each pair of concentrations assayed. Only values where the difference is greater than the 95% confidence interval of the measurements are included in the sum.

What is claimed is:

1. A method for treating in a human in need thereof a disease selected from the group of cancers, allergic disorders, autoimmune diseases, and inflammatory diseases, comprising administering to the human a therapeutically effective amount of a BTK inhibitor and a therapeutically effective amount of one or more inhibitor, wherein the BTK inhibitor is 6-amino-9-[(3R)-1-(2-butynoyl)-3-pyrrolidinyl]-7-(4-phenoxyphenyl)-7,9-dihydro-8H-purin-8-one, or a pharmaceutically acceptable salt or hydrate thereof, and
wherein the one or more inhibitor is selected from the group consisting of a JAK inhibitor, an ASK1 inhibitor, a BRD inhibitor, and a MMP9 inhibitor.

2. The method of claim 1 wherein the BTK inhibitor and/or the one or more inhibitor is administered intravenously, intramuscularly, parenterally, nasally or orally.

3. The method of claim 1 wherein the BTK inhibitor is administered prior, after or concurrently with the one or more inhibitor.

4. The method of claim 1 wherein the disease is selected from the group consisting of a hematologic malignancy, leukemia, lymphoma chronic lymphocytic leukemia (CLL), small lymphocytic lymphoma (SLL), non-Hodgkin's lymphoma, indolent non-Hodgkin's lymphoma (iNHL), mantle cell lymphoma, follicular lymphoma (FL), lymphoplasmacytic lymphoma, and marginal zone lymphoma, rheumatoid arthritis, systemic lupus erythematosus, chronic obstructive pulmonary disease (CORD), adult respiratory distress syndrome and asthma.

5. The method of claim 1 wherein the human is in refractory to at least one of the cancer therapies, or is in relapse after treatment with at least one anti-cancer therapy selected from the group consisting of: (a) fludarabine; (b) rituximab; (c) rituximab combined with fludarabine; (d) cyclophosphamide combined with fludarabine; (e) cyclophosphamide combined with rituximab and fludarabine; (f) cyclophosphamide combined with vincristine and prednisone; (g) cyclophosphamide combined with vincristine, prednisone, and rituximab; (h) a combination of cyclophosphamide, doxorubicin, vincristine, and prednisone; (i) Chlorambucil combined with prednisone, rituximab, obinutuzumab, or ofatumumab; (j) pentostatin combined with cyclophosphamide and rituximab, (k) bendamustine (Treanda®) combined with rituximab; (l) alemtuzumab; (m) fludarabine plus cyclophosphamide, bendamustine, or chlorambucil; and (n) fludarabine plus cyclophosphamide, bendamustine, or chlorambucil, combined with an anti-CD20 antibody.

6. A method for sensitizing a human who is (i) refractory to at least one chemotherapy treatment, or (ii) in relapse after treatment with chemotherapy, or both (i) and (ii), wherein the method comprises administering a Btk inhibitor in combination with an inhibitor to the human, and wherein the inhibitor is selected from the group consisting of a JAK inhibitor, an ASK1 inhibitor, a BRD inhibitor, and a MMP9 inhibitor.

7. The method of claims 1 or 6 wherein the JAK inhibitor is selected from the group consisting of momelotinib, filgotinib, 1-[1-[[3-fluoro-2-(trifluoromethyl)-4-pyridinyl]-4-piperidinyl]-3-[4-(7H-pyrrolo[2,3-d]pyrimidin-4-yl)-1H-pyrazol-1-yl]-3-azetidineacetonitrile, tofacitinib, oclacitinib, ruxolotinib, baracitinib, lestaurtinib, pacritinib, TG101348, JSI-124, GSK2585184, VX-509, INCB16562, XL019, NVP-BSK805, CEP33779, R-348, AC-430, CDP-R723, BMS911543, or a pharmaceutically acceptable salt thereof.

8. The method of claims 1 or 6 wherein the ASK1 inhibitor is 5-(4-cyclopropyl-1H-imidazol-1-yl)-2-fluoro-N-(6-(4-isopropyl-4H-1,2,4-triazol-3-yl)pyridin-2-yl)-4-methylbenzamide, or a pharmaceutically acceptable salt or hydrate thereof.

9. The method of claims 1 or 6 wherein the modulator of the bromodomain-containing protein is a compound of Formula II: 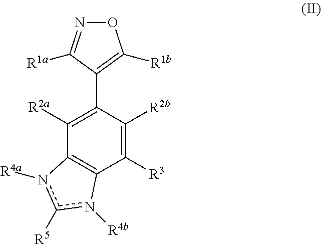
wherein
R^{1a} and R^{1b} are each independently C₁₋₆ alkyl optionally substituted with from 1 to 5 R²⁰ groups;
R^{2a} and R^{2b} are each independently H or halo;
R³ is
—C(O)OR^{a}, —NHC(O)OR^{a}, —NHS(O)₂R^{a}, or —S(O)₂NR^{a}R^{b}; or
selected from the group consisting of C₁₋₁₀ alkyl, C₁₋₁₀ alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R20 groups;
one of R^{4a} and R^{4b} is selected from the group consisting of H and C₁₋₆ alkyl optionally substituted with from 1 to 5 R²⁰ groups, and the other is absent;
R⁵ is —C(O)OR^{a}, —NHC(O)OR^{a}, —NHS(O)₂R^{a}, or —S(O)₂NR^{a}R^{b}; or
R⁵ is selected from the group consisting of H, C₁₋₁₀ alkyl, C₁₋₁₀ haloalkyl, C₁₋₁₀ alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups;
each R^{a} and R^{b} is independently selected from the group consisting of H, C₁₋₁₀ alkyl, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups; and
each R20 is independently selected from the group consisting of acyl, C₁₋₁₀ alkyl, C₁₋₁₀ alkoxy, amino, amido, amidino, C₅₋₁₀ aryl, C₆₋₂₀ arylalkyl, azido, carbamoyl, carboxyl, carboxyl ester, cyano, guanidino, halo, C₁₋₁₀ haloalkyl, C₁₋₁₀ heteroalkyl, C₅₋₁₀ heteroaryl, C₆₋₂₀ heteroarylalkyl, hydroxy, hydrazino, imino, oxo, nitro, sulfinyl, sulfonic acid, sulfonyl, thiocyanate, thiol, and thione; and
wherein the C₁₋₁₀ alkyl, C₅₋₁₀ aryl, C₆₋₂₀ arylalkyl, C₁₋₁₀ heteroalkyl, C₅₋₁₀ heteroaryl, and C₆₋₂₀ heteroarylalkyl groups are optionally substituted with from 1 to 3 substituents independently selected from C₁₋₆ alkyl, C₅₋₁₀ aryl, halo, C₁₋₆ haloalkyl, cyano, hydroxy, and C₁₋₆ alkoxy;
or a pharmaceutically acceptable salt thereof.

10. The method of claims 1 or 6 wherein the MMP9 inhibitor comprises a MMP9 binding protein comprising:
an immunoglobulin heavy chain polypeptide, or a functional fragment thereof; and
an immunoglobulin light chain polypeptide, or a functional fragment thereof; wherein the MMP9 binding protein specifically binds to human MMP9, and
wherein the MMP9 binding protein competes for binding to human MMP9 with an antibody comprising heavy chain CDRs of SEQ ID NOs: 13-15 or light chain CDRs of SEQ ID Nos. 16-18.

11. The method of claim 10 wherein the immunoglobulin heavy chain comprises an amino acid sequence SEQ ID NO. 7 and wherein the immunoglobulin light chain polypeptide or functional fragment thereof comprises an amino acid sequence SEQ ID NO. 12.

12. An article of manufacture comprising:
a unit dosage form of a BTK inhibitor, wherein the BTK inhibitor is 6-amino-9-[(3R)-1-(2-butynoyl)-3-pyrrolidinyl]-7-(4-phenoxyphenyl)-7,9-dihydro-8H-purin-8-one, or a pharmaceutically acceptable salt or hydrate thereof; and a unit dosage form of one or more inhibitor, wherein the inhibitor is selected from the group consisting a JAK inhibitor, an ASK1 inhibitor, a BRD inhibitor, and a MMP9 inhibitor;
a label containing instructions for use in treating a disease selected from the group of cancers, allergic disorders, autoimmune diseases, and inflammatory diseases.

13. A kit comprising:
a pharmaceutical composition comprising a pharmaceutically effective amount of a BTK inhibitor, wherein the BTK inhibitor is 6-amino-9-[(3R)-1-(2-butynoyl)-3-pyrrolidinyl]-7-(4-phenoxyphenyl)-7,9-dihydro-8H-purin-8-one, or a pharmaceutically acceptable salt or hydrate thereof;
a pharmaceutical composition comprising a pharmaceutically effective amount of one or more inhibitor, wherein the inhibitor is selected from the group consisting a JAK inhibitor, an ASK1 inhibitor, a BRD inhibitor, and a MMP9 inhibitor, and
instructions for use in treating a disease selected from the group of a cancer, allergic disorders, autoimmune diseases, and inflammatory diseases.
